(19) United States
(12) Patent Application Publication
(10) Pub. No.: US 2024/0215444 A1
(43) Pub. Date: Jun. 27, 2024

(54) LIGHT EMITTING ELEMENT AND POLYCYCLIC COMPOUND FOR THE SAME

(71) Applicant: Samsung Display Co., Ltd., Yongin-si (KR)

(72) Inventor: Takao SAKUMA, Yokohama (JP)

(21) Appl. No.: 18/501,961

(22) Filed: Nov. 3, 2023

(30) Foreign Application Priority Data
Dec. 6, 2022 (KR) 10-2022-0169163

(51) Int. Cl. H10K 85/60; C07D 405/14; C07D 409/14; C09K 11/06

(52) U.S. Cl. CPC H10K 85/6574; C07D 405/14; C07D 409/14; C09K 11/06; H10K 85/6572; H10K 85/6576; H10K 50/15

(57) ABSTRACT

A light emitting element includes a first electrode, a second electrode on the first electrode, an emission layer between the first electrode and the second electrode, and a hole transport region between the first electrode and the emission layer. The hole transport region includes a polycyclic compound represented by Formula 1. The polycyclic compound is configured so that the hole transport properties of the hole transport region and the power efficiency of the light emitting element may be enhanced or improved.

### CROSS-REFERENCE TO RELATED APPLICATION

This application claims priority to and the of Korean Patent Application No. 10-2022-0169163, filed on Dec. 6, 2022, in the Korean Intellectual Property Office, the entire content of which is hereby incorporated by reference.

### BACKGROUND

### 1. Field

One or more aspects of embodiments of the present disclosure relate to a light emitting element, for example, a light emitting element with improved power efficiency, a polycyclic compound utilized therein and a display device including the light emitting element.

### 2. Description of the Related Art

Recently, the development of organic electroluminescence display devices and/or the like to be utilized in an image display device has been actively conducted. The organic electroluminescence display devices and/or the like may include a “self-luminescent light emitting element” that enables display of images by recombining holes and electrons injected from a first electrode and a second electrode in an emission layer. Subsequently, a light emitting material located in the emission layer emits light to accomplish display.

Implementation of the organic electroluminescence device in a display device requires (or there is a desire) that the light emitting element (e.g., self-luminescent light emitting element) possess reduced driving voltage, improved power efficiency, and/or a long lifespan. Therefore, the need exists for development of a material for a light emitting element which is capable of stably (or suitably) implementing these properties. For example, in an effort to implement a light emitting element having high power efficiency, the development of materials for a hole transport region having excellent or suitable hole transport properties is being pursued.

### SUMMARY

One or more aspects of embodiments of the present disclosure are directed toward a light emitting element having increased power efficiency.

One or more aspects of embodiments of the present disclosure are directed toward a polycyclic compound as a material for utilization in a light emitting element, which is capable of improving power efficiency.

An embodiment of the present disclosure relates to a light emitting element including a first electrode, a second electrode located or disposed on the first electrode, an emission layer located or disposed between the first electrode and the second electrode, and a hole transport region located or disposed between the first electrode and the emission layer. In an embodiment, the hole transport region includes a polycyclic compound represented by Formula 1.

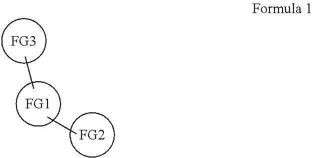

In Formula 1, FG1 is represented by Formula 1-1, FG2 is represented by Formula 1-2, and FG3 is represented by any one among Formulas 1-3 to 1-5.

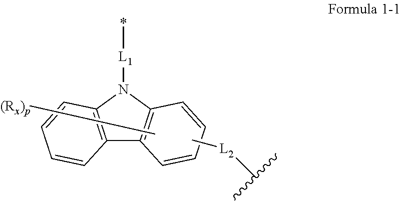

In Formula 1-1, p is an integer of 0 to 7, L1 is a direct linkage or a substituted or unsubstituted arylene group having 6 to 30 ring-forming carbon atoms, L2 is a direct linkage or a substituted or unsubstituted arylene group having 6 to 30 ring-forming carbon atoms, Rₓ is a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, “ [symbol] ” is a portion bonded to FG2, and “ [symbol] ” is a portion bonded to FG3.

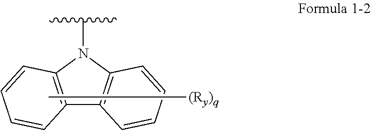

In Formula 1-2, q is an integer of 0 to 8, R_{y} is a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and “ [symbol] ” is a portion bonded to FG1.

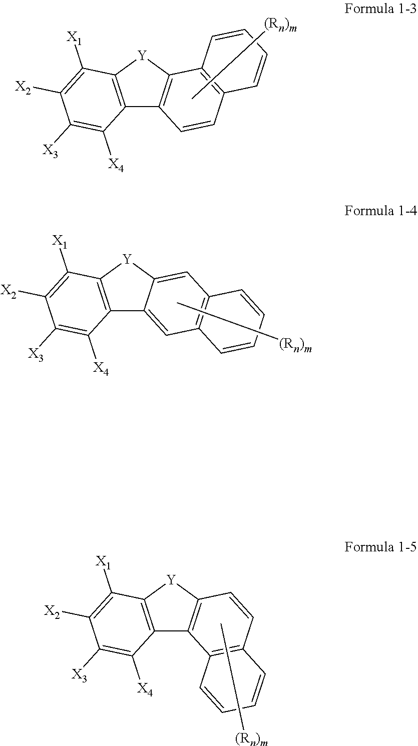

In Formulas 1-3 to 1-5, m is an integer of 0 to 6, Rₙ is a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and Y is O or S. In an embodiment, at least one selected from X₁ to X₄ each independently may be a portion bonded to FG1 of Formula 1, a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and at least one of X₁ to X₄ is a portion bonded to FG1 of Formula 1.

In an embodiment, Formula 1 may be represented by any one among Formulas 2-1 to 2-3.

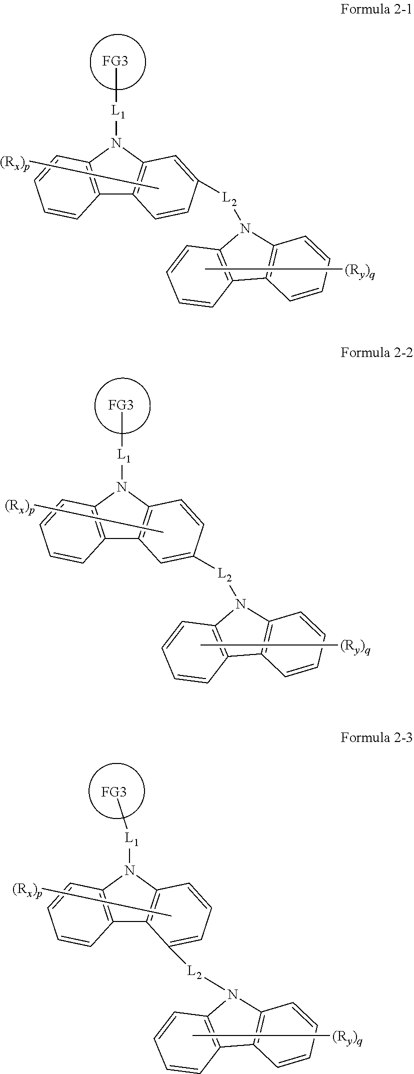

In Formulas 2-1 to 2-3, FG3, L₁, L₂, Rₓ, R_{y}, p, and q may each independently be the same as defined in Formula 1.

In an embodiment, Formula 1 may be represented by Formula 3-1 or

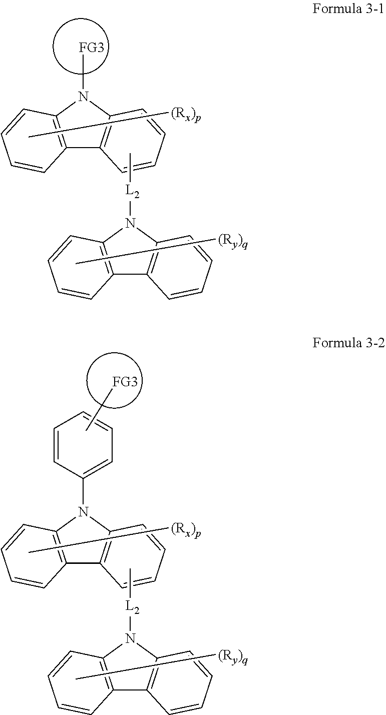

In Formulas 3-1 and 3-2, FG3, L₂, Rₓ, R_{y}, p, and q may each independently be the same as defined in Formula 1.

In an embodiment, Formula 3-2 may be represented by Formula 3A or Formula 3B.

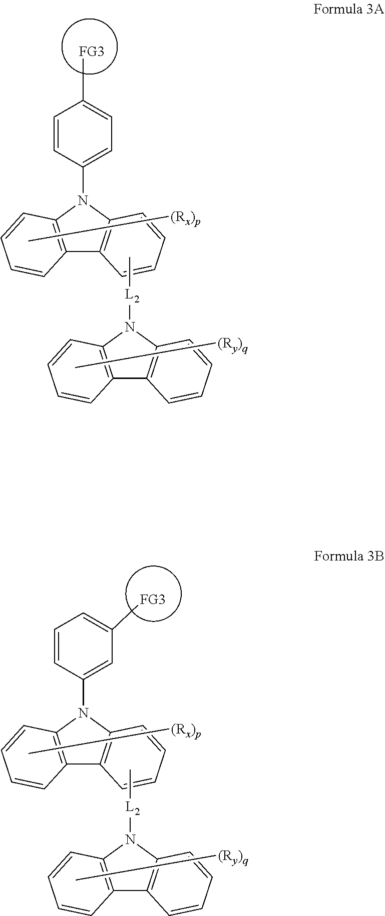

In Formulas 3A and 3B, FG3, L₂, Rₓ, R_{y}, p, and q may each independently be the same as defined in Formula 1.

In an embodiment, Formula 1 may be represented by Formula 4-1 or Formula 4-2.

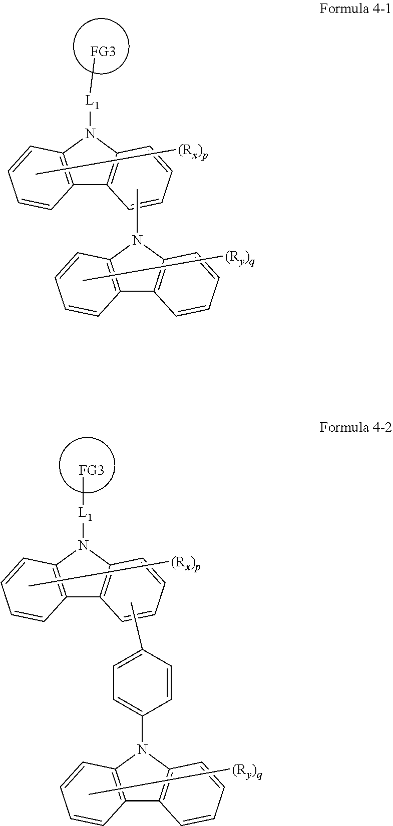

In Formulas 4-1 and 4-2, FG3, L₁, Rₓ, R_{y}, p, and q may each independently be the same as defined in Formula 1.

In an embodiment, in Formula 1-1, Rₓ may be a hydrogen atom or an unsubstituted phenyl group.

In an embodiment, in Formula 1-2, R_{y} may be a hydrogen atom, a deuterium atom, a deuterium-substituted phenyl group, an unsubstituted phenyl group, an unsubstituted naphthyl group, an unsubstituted dibenzofuran group, or an unsubstituted dibenzothiophene group.

In an embodiment, in each of Formulas 1-3 to 1-5, each X₁ to X₄ that is not bonded to FG1 of Formula 1 may be a hydrogen atom.

In an embodiment, the hole transport region may include a hole injection layer located or disposed on the first electrode, and a hole transport layer located or disposed between the hole injection layer and the emission layer. In an embodiment, the hole transport layer may include the polycyclic compound.

In an embodiment, the emission layer may be configured to emit blue light.

In an embodiment, the emission layer may be configured to emit light of fluorescence (i.e., fluorescence light).

In an embodiment, in Formula 1, FG1 may be represented by any one among B-1 to B-7.

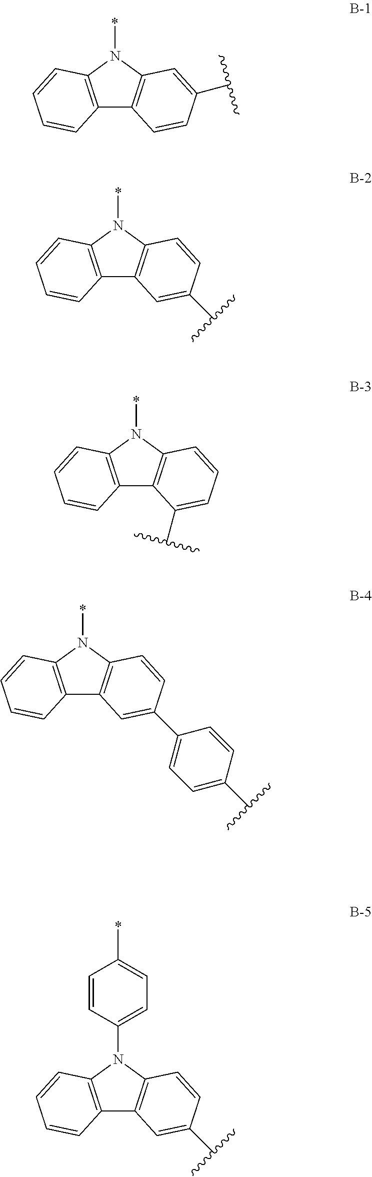 [SMILES: *c1ccc2c3ccccc3n(*)c2c1 ; *c1ccc2c(c1)c1ccccc1n2* ; *c1cccc2c1c1ccccc1n2* ; *c1ccc(-c2ccc3c(c2)c2ccccc2n3*)cc1 ; *c1ccc(-n2c3ccccc3c3cc(*)ccc32)cc1]

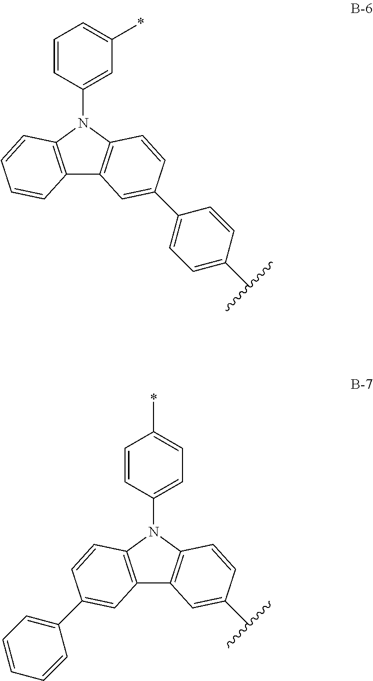 [SMILES: *c1ccc(-c2ccc3c(c2)c2ccccc2n3-c2cccc(*)c2)cc1 ; *c1ccc(-n2c3ccc(*)cc3c3cc(-c4ccccc4)ccc32)cc1]

In an embodiment, in Formula 1, FG2 may be represented by any one among C-1 to C-8.

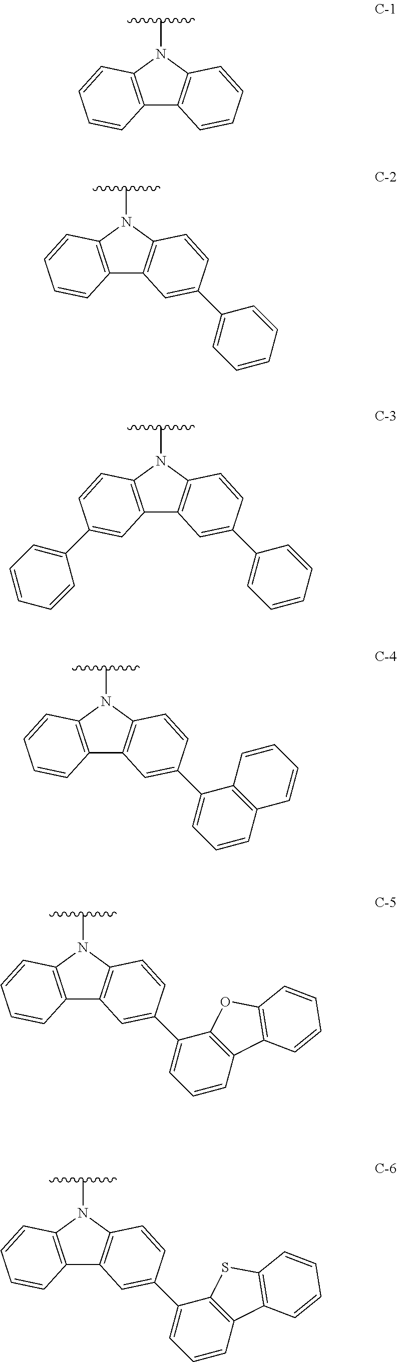 [SMILES: *n1c2ccccc2c2ccccc21 ; *n1c2ccccc2c2cc(-c3ccccc3)ccc21 ; *n1c2ccc(-c3ccccc3)cc2c2cc(-c3ccccc3)ccc21 ; *n1c2ccccc2c2cc(-c3cccc4ccccc34)ccc21 ; *n1c2ccccc2c2cc(-c3cccc4c3oc3ccccc34)ccc21 ; *n1c2ccccc2c2cc(-c3cccc4c3sc3ccccc34)ccc21]

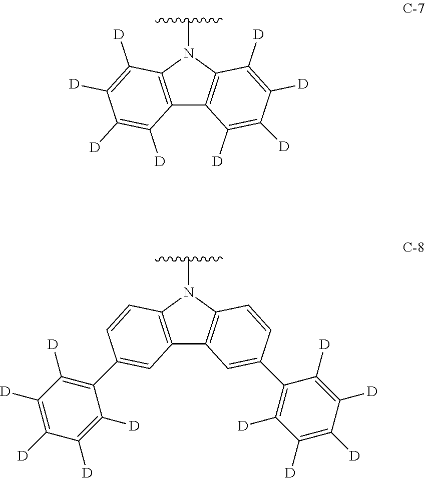 [SMILES: *n1c2c([2H])c([2H])c([2H])c([2H])c2c2c([2H])c([2H])c([2H])c([2H])c21 ; *n1c2ccc(-c3c([2H])c([2H])c([2H])c([2H])c3[2H])cc2c2cc(-c3c([2H])c([2H])c([2H])c([2H])c3[2H])ccc21]

In an embodiment, in Formula 1, FG1 may be represented by any one among B-1 to B-7, and FG2 may be represented by any one among C-1 to C-8.

An embodiment of the present disclosure relates to a polycyclic compound represented by Formula 1.

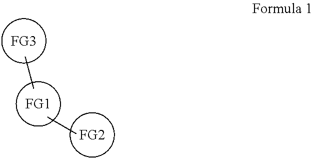

In Formula 1, FG1 is represented by Formula 1-1, FG2 is represented by Formula 1-2, and FG3 is represented by any one among Formulas 1-3 to 1-5.

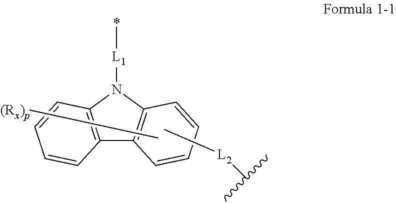

In Formula 1-1, p is an integer of 0 to 7, L₁ is a direct linkage or a substituted or unsubstituted arylene group having 6 to 30 ring-forming carbon atoms, L₂ is a direct linkage or a substituted or unsubstituted arylene group having 6 to 30 ring-forming carbon atoms, Rₓ is a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, “ [symbol] ” is a portion bonded to FG2, and “ [symbol] ” is a portion bonded to FG3.

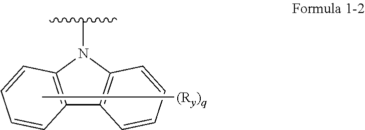

In Formula 1-2, q is an integer of 0 to 8, R_{y} is a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and “ [symbol] ” is a portion bonded to FG1.

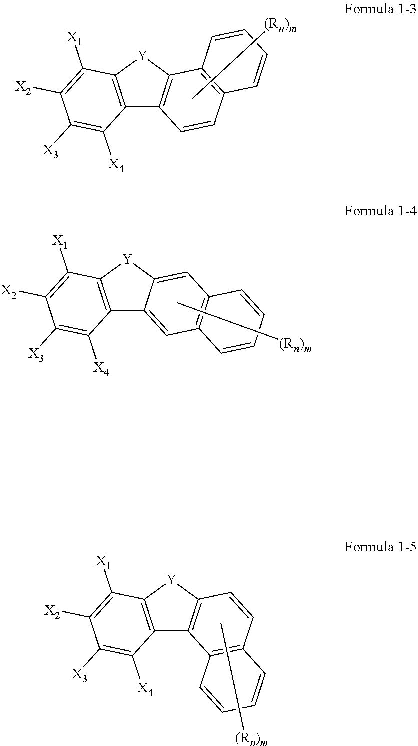

In Formulas 1-3 to 1-5, m is an integer of 0 to 6, Rₙ is a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and Y is O or S. In an embodiment, X₁ to X₄ each independently may be a portion bonded to FG1 of Formula 1, a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and at least one of X₁ to X₄ is a portion bonded to FG1 of Formula 1.

In an embodiment, Formula 1 may be represented by any one among Formulas 2-1 to 2-3.

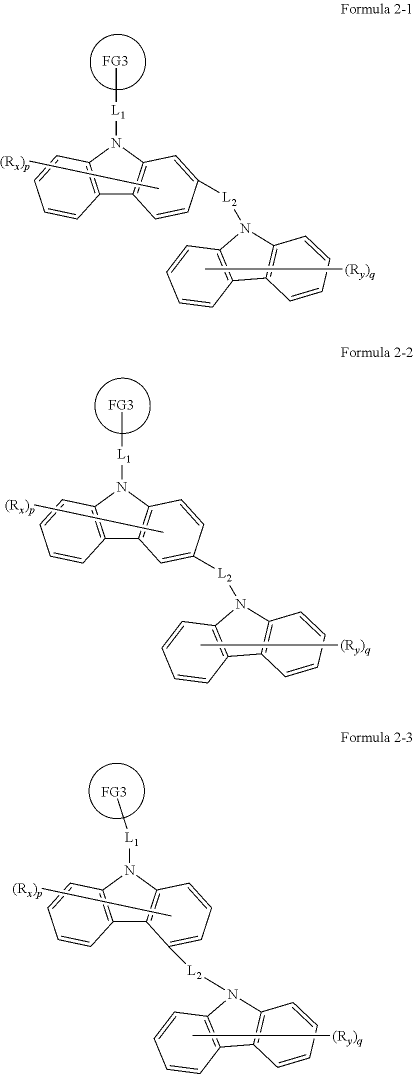

In Formulas 2-1 to 2-3, FG3, L₁, L₂, Rₓ, R_{y}, p, and q may each independently be the same as defined in Formula 1.

In an embodiment, Formula 1 may be represented by Formula 3-1 or

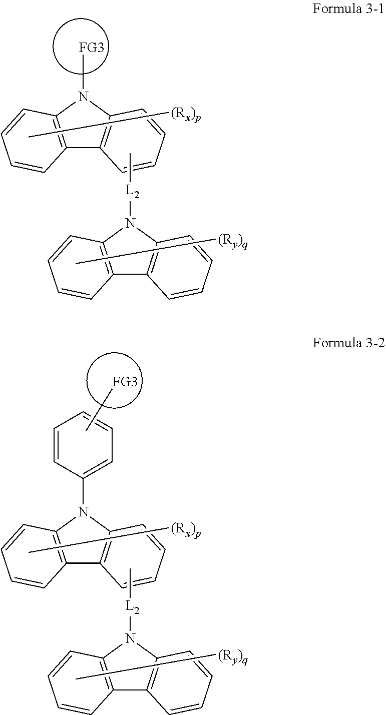

In Formulas 3-1 and 3-2, FG3, L₂, Rₓ, R_{y}, p, and q may each independently be the same as defined in Formula 1.

In an embodiment, Formula 3-2 may be represented by Formula 3A or Formula 3B.

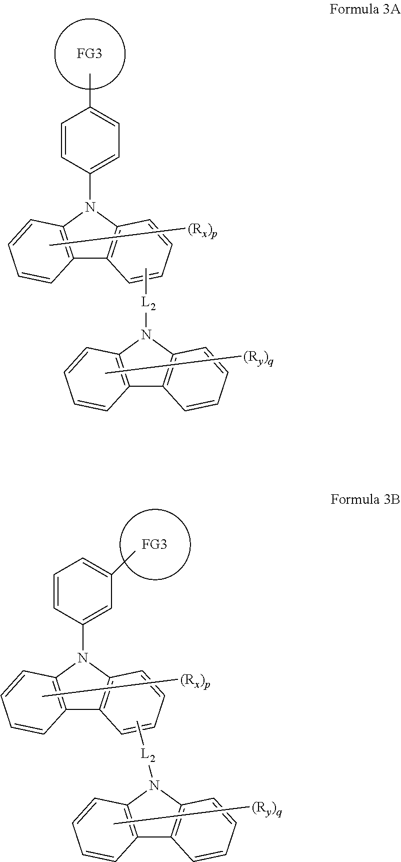

In Formulas 3A and 3B, FG3, L₂, Rₓ, R_{y}, p, and q may each independently be the same as defined in Formula 1.

In an embodiment, Formula 1 may be represented by Formula 4-1 or Formula 4-2.

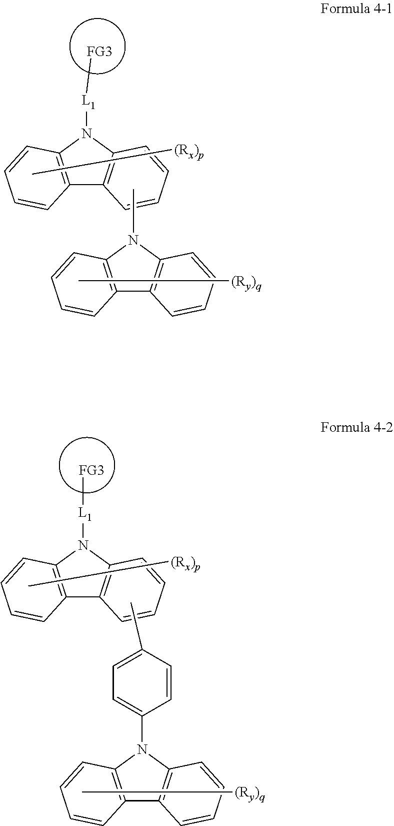

In Formulas 4-1 and 4-2, FG3, L₁, Rₓ, R_{y}, p, and q may each independently be the same as defined in Formula 1.

In an embodiment, in Formula 1-1, Rₓ may be a hydrogen atom or an unsubstituted phenyl group.

In an embodiment, in Formula 1-2, R_{y} may be a hydrogen atom, a deuterium atom, a deuterium-substituted phenyl group, an unsubstituted phenyl group, an unsubstituted naphthyl group, an unsubstituted dibenzofuran group, or an unsubstituted dibenzothiophene group.

In an embodiment, in each of Formulas 1-3 to 1-5, each X₁ to X₄ that is not bonded to FG1 of Formula 1 may be a hydrogen atom.

In an embodiment, in Formula 1, FG1 may be represented by any one among B-1 to B-7.

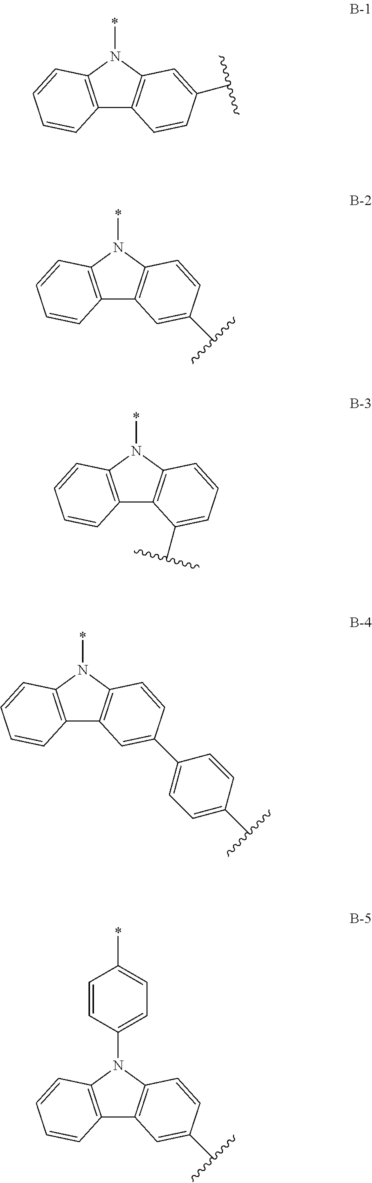 [SMILES: *c1ccc2c3ccccc3n(*)c2c1 ; *c1ccc2c(c1)c1ccccc1n2* ; *c1cccc2c1c1ccccc1n2* ; *c1ccc(-c2ccc3c(c2)c2ccccc2n3*)cc1 ; *c1ccc(-n2c3ccccc3c3cc(*)ccc32)cc1]

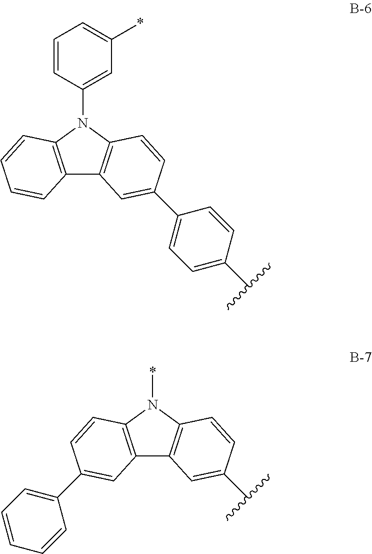 [SMILES: *c1ccc(-c2ccc3c(c2)c2ccccc2n3-c2cccc(*)c2)cc1 ; *c1ccc2c(c1)c1cc(-c3ccccc3)ccc1n2*]

In an embodiment, in Formula 1, FG2 may be represented by any one among C-1 to C-8.

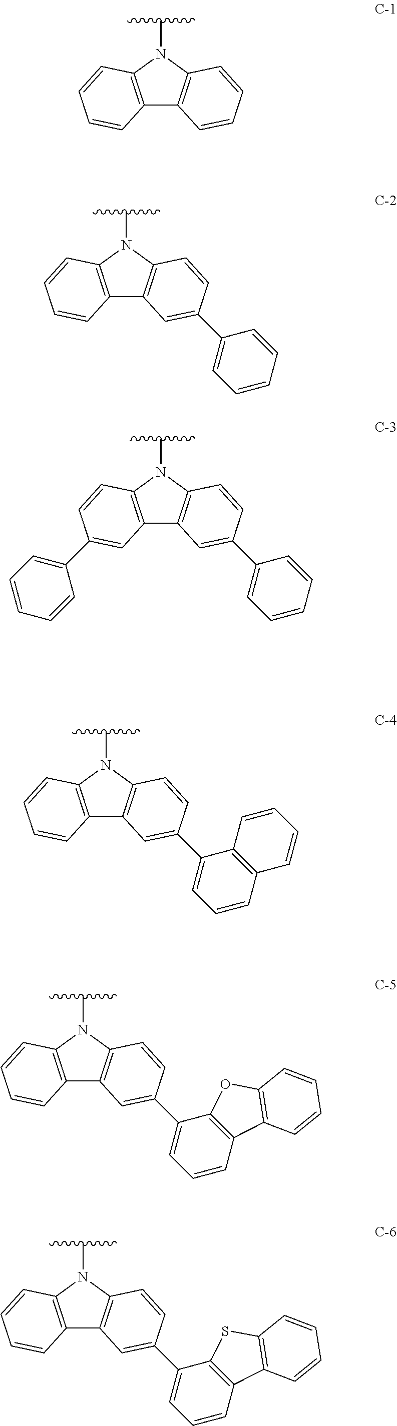 [SMILES: *n1c2ccccc2c2ccccc21 ; *n1c2ccccc2c2cc(-c3ccccc3)ccc21 ; *n1c2ccc(-c3ccccc3)cc2c2cc(-c3ccccc3)ccc21 ; *n1c2ccccc2c2cc(-c3cccc4ccccc34)ccc21 ; *n1c2ccccc2c2cc(-c3cccc4c3oc3ccccc34)ccc21 ; *n1c2ccccc2c2cc(-c3cccc4c3sc3ccccc34)ccc21]

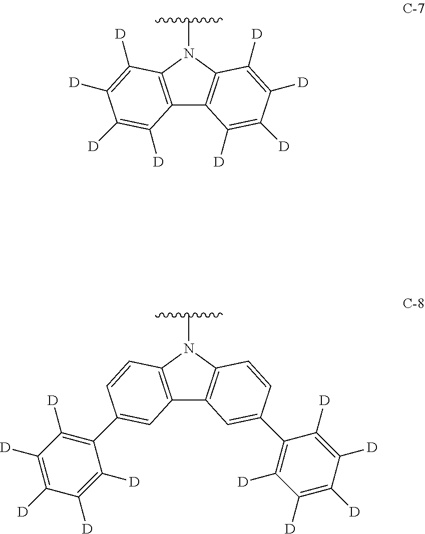 [SMILES: *n1c2c([2H])c([2H])c([2H])c([2H])c2c2c([2H])c([2H])c([2H])c([2H])c21 ; *n1c2ccc(-c3c([2H])c([2H])c([2H])c([2H])c3[2H])cc2c2cc(-c3c([2H])c([2H])c([2H])c([2H])c3[2H])ccc21]

In an embodiment, in Formula 1, FG1 may be represented by any one among B-1 to B-7, and FG2 may be represented by any one among C-1 to C-8.

### BRIEF DESCRIPTION OF THE DRAWINGS

The accompanying drawings are included to provide a further understanding of the present disclosure, and are incorporated in and constitute a part of this specification. The drawings illustrate example embodiments of the present disclosure and, together with the description, serve to explain principles of the present disclosure. In the drawings:

FIG. 1 is a plan view of a display device according to one or more embodiments of the present disclosure;

FIG. 2 is a cross-sectional view of a display device according to one or more embodiments of the present disclosure;

FIG. 3 is a cross-sectional view schematically showing a light emitting element according to one or more embodiments of the present disclosure;

FIG. 4 is a cross-sectional view schematically showing a light emitting element according to one or more embodiments of the present disclosure;

FIG. 5 is a cross-sectional view schematically showing a light emitting element according to one or more embodiments of the present disclosure;

FIG. 6 is a cross-sectional view schematically showing a light emitting element according to one or more embodiments of the present disclosure;

FIG. 7 is a cross-sectional view of a display device according to one or more embodiments of the present disclosure;

FIG. 8 is a cross-sectional view of a display device according to one or more embodiments of the present disclosure;

FIG. 9 is a cross-sectional view showing a display device according to one or more embodiments of the present disclosure;

FIG. 10 is a cross-sectional view showing a display device according to one or more embodiments of the present disclosure; and

FIG. 11 is a schematic perspective view of an electronic device including a display device according to one or more embodiments of the present disclosure.

### DETAILED DESCRIPTION

The present disclosure may be modified in many alternate forms, and thus specific embodiments will be exemplified in the drawings and described in more detail. It should be understood, however, that it is not intended to limit the present disclosure to the particular forms disclosed, but rather, is intended to cover all modifications, equivalents, and alternatives falling within the spirit and scope of the present disclosure. Unless otherwise defined, all chemical names, technical and scientific terms, and terms defined in common dictionaries should be interpreted as having meanings consistent with the context of the related art, and should not be interpreted in an ideal or overly formal sense.

In describing the drawings, like reference numerals are utilized for like elements. In the drawings, the sizes of elements may be exaggerated for clarity. It will be understood that, although the terms first, second, etc. may be utilized herein to describe one or more suitable elements, these elements should not be limited by these terms. These terms are only utilized to distinguish one element from another element. For example, a first element may be referred to as a second element, and similarly, a second element may be referred to as a first element without departing from the teachings of the present disclosure. The singular forms are intended to include the plural forms as well, unless the context clearly indicates otherwise.

In the present description, it should be understood that the terms “comprise,” “comprises,” “comprising,” “has,” “have,” “having,” “includes,” and/or “including” are intended to specify the presence of stated features, integers, steps, operations, elements, components, or combinations thereof in the disclosure, but do not preclude the presence or addition of one or more other features, integers, steps, operations, elements, components, or combinations thereof.

As used herein, the terms “use,” “using,” and “used” may be considered synonymous with the terms “utilize,” “utilizing,” and “utilized,” respectively.

As used herein, expressions such as “at least one of,” “one of,” and “selected from,” when preceding a list of elements, modify the entire list of elements and do not modify the individual elements of the list. For example, the expressions “at least one of a to c,” “at least one of a, b or c,” and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

As used herein, the term “and/or” includes any and all combinations of one or more of the associated listed items.

The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

It will be understood that when an element is referred to as being “on,” “connected to,” or “coupled to” another element, it may be directly on, connected, or coupled to the other element or one or more intervening elements may also be present. When an element is referred to as being “directly on,” “directly connected to,” or “directly coupled to” another element, there are no intervening elements present.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the drawings. It will be understood that the spatially relative terms are intended to encompass different orientations of the device in use or operation in addition to the orientation depicted in the drawings. For example, if the device in the drawings is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

Unless otherwise defined, all terms (including technical and scientific terms) used herein have the same meaning as commonly understood by those of ordinary skill in the art to which the present disclosure pertains. It is also to be understood that terms defined in commonly used dictionaries should be interpreted as having meanings consistent with meanings in the context of the related art, unless expressly defined herein, and should not be interpreted in an ideal or overly formal sense.

In this context, “consisting essentially of” means that any additional components will not materially affect the chemical, physical, optical, or electrical properties of the semiconductor film.

Further, in this specification, the phrase “on a plane,” or “plan view,” means viewing a target portion from the top, and the phrase “on a cross-section” means viewing a cross-section formed by vertically cutting a target portion from the side.

### Definitions

In the present description, the term “substituted or unsubstituted” may indicate that one is substituted or unsubstituted with at least one substituent selected from the group consisting of a deuterium atom, a halogen atom, a cyano group, a nitro group, an amino group, a silyl group, oxy group, thio group, sulfinyl group, sulfonyl group, carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the substituents exemplified herein may be substituted or unsubstituted. For example, a biphenyl group may be interpreted as an aryl group or as a phenyl group substituted with a phenyl group.

In the present description, the term “bonded to an adjacent group to form a ring” may indicate that one is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In some embodiments, the rings formed by being bonded to each other may be connected to another ring to form a spiro structure.

In the present description, the term “an adjacent group” may refer to a substituent substituted for an atom which is directly connected to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as mutually “adjacent groups” and two ethyl groups in 1,1-diethylcyclopentane may be interpreted as mutually “adjacent groups”. In some embodiments, two methyl groups in 4,5-dimethylphenanthrene may be interpreted as mutually “adjacent groups”.

In the present description, examples of a halogen atom may include a fluorine atom, a chlorine atom, a bromine atom, and an iodine atom.

In the present description, an alkyl group may be a linear, branched or cyclic type or kind. The number of carbon atoms in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, a s-butyl group, a t-butyl group, an i-butyl group, a 2-ethylbutyl group, a 3,3-a dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but are not limited thereto.

In the present description, an alkyl group may be linear or branched. The number of carbon atoms in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, a s-butyl group, a t-butyl group, an i-butyl group, a 2-ethylbutyl group, a 3,3-a dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but are not limited thereto.

In the present description, a cycloalkyl group may refer to a cyclic alkyl group. The number of carbon atoms in the cycloalkyl group is 3 to 50, 3 to 30, 3 to 20, or 3 to 10. Examples of the cycloalkyl group include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like, but are not limited thereto.

In the present description, an alkenyl group refers to a hydrocarbon group including at least one carbon double bond in the middle or end of an alkyl group having 2 or more carbon atoms. The alkenyl group may be linear or branched. The number of carbon atoms is not particularly limited, but may be 2 to 30, 2 to 20, or 2 to 10. Examples of the alkenyl group include a vinyl group, a 1-butenyl group, a 1-pentenyl group, a 1,3-butadienyl aryl group, a styrenyl group, a styryl vinyl group, and/or the like, but are not limited thereto.

In the present description, an alkynyl group refers to a hydrocarbon group including at least one carbon triple bond in the middle or end of an alkyl group having 2 or more carbon atoms. The alkynyl group may be linear or branched. The number of carbon atoms is not particularly limited, but may be 2 to 30, 2 to 20, or 2 to 10. Specific examples of the alkynyl group may include an ethynyl group, a propynyl group, and/or the like, but are not limited thereto.

In the present description, a hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the present description, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, and/or the like, but are not limited thereto.

In the present description, a fluorenyl group may be substituted, and two substituents may be bonded to each other to form a spiro structure. An example that the fluorenyl group is substituted is as follows. However, the embodiment of the present disclosure is not limited thereto.

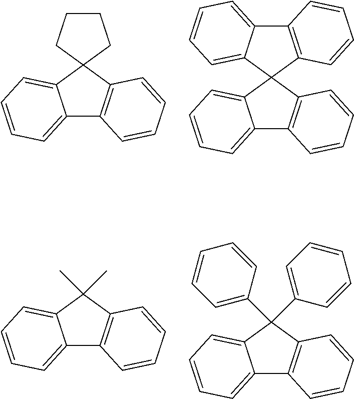

In the present description, a heterocyclic group refers to any functional group or substituent derived from a ring containing at least one of B, O, N, P, Si, or S as a hetero atom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

In the present description, the heterocyclic group may contain at least one of B, O, N, P, Si or S as a hetero atom. When the heterocyclic group contains two or more hetero atoms, the two or more hetero atoms may be the same as or different from each other. The heterocyclic group may be a monocyclic heterocyclic group or a polycyclic heterocyclic group, and include a heteroaryl group. The number of ring-forming carbon atoms in the heterocyclic group may be 2 to 30, 2 to 20, or 2 to 10.

In the present description, the aliphatic heterocyclic group may contain at least one of B, O, N, P, Si, or S as a hetero atom. The number of ring-forming carbon atoms in the aliphatic heterocyclic group may be 2 to 30, 2 to 20, or 2 to 10. Examples of the aliphatic heterocyclic group include an oxirane group, a thiirane group, a pyrrolidine group, a piperidine group, a tetrahydrofuran group, a tetrahydrothiophene group, a thiane group, a tetrahydropyran group, a 1,4-dioxane group, and/or the like, but are not limited to thereto.

In the present description, a heteroaryl group may include at least one of B, O, N, P, Si, or S as a hetero atom. When the heteroaryl group contains two or more hetero atoms, the two or more hetero atoms may be the same as or different from each other. The heteroaryl group may be a monocyclic heteroaryl group or a polycyclic heteroaryl group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and/or the like, but are not limited thereto.

In the present description, the description of the aryl group may be applied to an arylene group, except that the arylene group is a divalent group. The description of the heteroaryl group may be applied to a heteroarylene group, except that the heteroarylene group is a divalent group.

In the present description, a silyl group includes an alkyl silyl group and an aryl silyl group. Examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and/or the like, but are not limited thereto.

In the present description, the number of carbon atoms in an amino group is not particularly limited, but may be 1 to 30. The amino group may include an alkyl amino group, an aryl amino group, or a heteroaryl amino group. Examples of the amino group include a methylamino group, a dimethylamino group, a phenylamino group, a diphenylamino group, a naphthylamino group, a 9-methyl-anthracenylamino group, a triphenylamino group, and/or the like, but are not limited thereto.

In the present description, the number of carbon atoms in a carbonyl group is not particularly limited, but may be 1 to 40, 1 to 30, or 1 to 20. For example, the carbonyl group may have one or more of the following structure, but is not limited thereto.

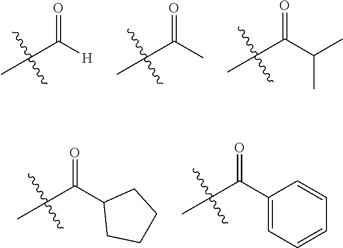

In the present description, the number of carbon atoms in a sulfinyl group and a sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

In the present description, a thio group may include an alkyl thio group and an aryl thio group. The thio group may indicate the one that a sulfur atom is bonded to an alkyl group or an aryl group as defined herein. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, and/or the like, but are not limited to thereto.

In the present description, an oxy group may indicate the one that an oxygen atom is bonded to an alkyl group or aryl group as defined herein. The oxy group may include an alkoxy group and an aryl oxy group. The alkoxy group may be linear, branched or cyclic. The number of carbon atoms in the alkoxy group is not particularly limited, but may be, for example, 1 to 20, or 1 to 10. Examples of the oxy group include methoxy, ethoxy, n-propoxy, isopropoxy, butoxy, pentyloxy, hexyloxy, octyloxy, nonyloxy, decyloxy, benzyloxy, and/or the like, but are not limited thereto.

In the present description, a boron group may refer to one that a boron atom is bonded to an alkyl group or aryl group as defined herein. The boron group includes an alkyl boron group and an aryl boron group. Examples of the boron group include a dimethyl boron group, a diethyl boron group, a t-butylmethyl boron group, a diphenyl boron group, a phenyl boron group, and/or the like, but are not limited thereto.

In the present description, an alkenyl group may be linear or branched. The number of carbon atoms is not particularly limited, but may be 2 to 30, 2 to 20, or 2 to 10. Examples of the alkenyl group include a vinyl group, a 1-butenyl group, a 1-pentenyl group, a 1,3-butadienyl aryl group, a styrenyl group, a styryl vinyl group, and/or the like, but are not limited thereto.

In the present description, the number of carbon atoms in an amine group is not particularly limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, a triphenylamine group, and/or the like, but are not limited thereto.

In the present description, examples of the alkyl group include an alkylthio group, an alkyl sulfoxy group, an alkylaryl group, an alkylamino group, an alkyl boron group, an alkyl silyl group, and an alkyl amine group.

In the present description, examples of the aryl group include an aryloxy group, an arylthio group, an aryl sulfoxy group, an arylamino group, an aryl boron group, an aryl silyl group, and an aryl amine group.

In the present description, a direct linkage may refer to a single bond.

In some embodiments, in the present description, “ [symbol] ” and “ [symbol] ” refers to a position to be connected.

Hereinafter, embodiments of the present disclosure will be described with reference to the accompanying drawings.

### Display Device

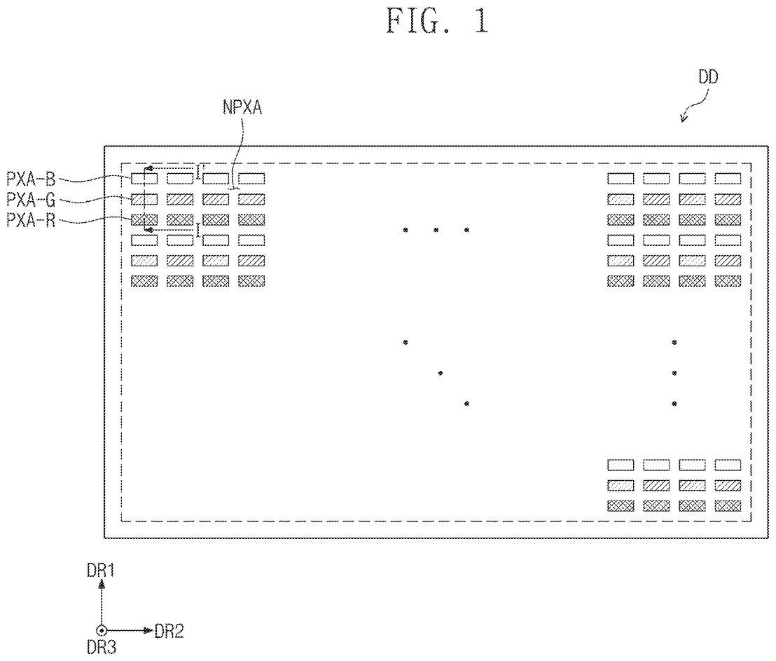

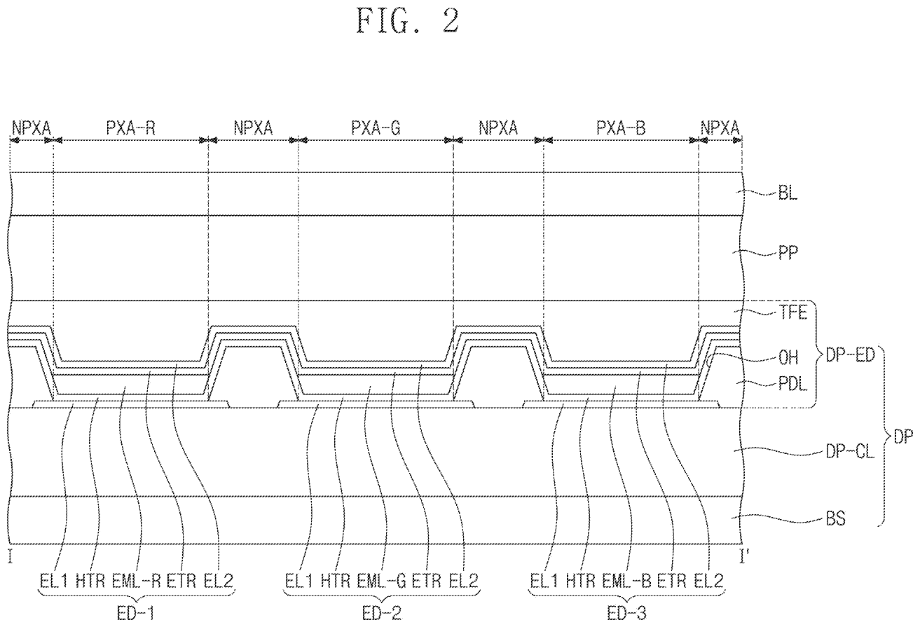

FIG. 1 is a plan view showing an embodiment of a display device DD. FIG. 2 is a cross-sectional view of a display device DD of an embodiment showing a portion corresponding to line I-I′ of FIG. 1.

The display device DD may include a display panel DP and an optical layer PP located or disposed on the display panel DP. The display panel DP includes light emitting elements ED-1, ED-2, and ED-3. The display device DD may include a plurality of light emitting elements ED-1, ED-2, and ED-3. The optical layer PP may be located or disposed on the display panel DP to control reflected light in the display panel DP due to external light. The optical layer PP may include, for example, a polarizing layer or a color filter layer. In some embodiments, unlike what is shown in the drawings (e.g., FIG. 2), the optical layer PP may not be included in the display device DD of an embodiment.

A base substrate BL may be located or disposed on the optical layer PP. The base substrate BL may be a member providing a base surface on which the optical layer PP is located or disposed. The base substrate BL may be a glass substrate, a metal substrate, a plastic substrate, and/or the like. However, the embodiment of the present disclosure is not limited thereto, and the base substrate BL may be an inorganic layer, an organic layer, and/or a composite material layer. In some embodiments, unlike what is shown, the base substrate BL may not be provided in an embodiment.

The display device DD according to an embodiment may further include a filling layer. The filling layer may be located or disposed between a display element layer DP-ED and the base substrate BL. The filling layer may be an organic material layer. The filling layer may include an acrylic resin, a silicone-based resin, and/or an epoxy-based resin.

The display panel DP may include a base layer BS, a circuit layer DP-CL located or provided on the base layer BS, and a display element layer DP-ED. The display element layer DP-ED may include two or more pixel defining films PDL, a plurality of light emitting elements ED-1, ED-2, and ED-3 located or disposed between the pixel defining films PDL, and an encapsulation layer TFE located or disposed on the plurality of light emitting elements ED-1, ED-2, and ED-3.

The base layer BS may be a member providing a base surface in which the display element layer DP-ED is located or disposed. The base layer BS may be a glass substrate, a metal substrate, a plastic substrate, and/or the like. However, the embodiment of the present disclosure is not limited thereto, and the base layer BS may be an inorganic layer, an organic layer, or a composite material layer.

In an embodiment, the circuit layer DP-CL may be located or disposed on the base layer BS, and the circuit layer DP-CL may include a plurality of transistors. The transistors may each include a control electrode, an input electrode, and an output electrode. For example, the circuit layer DP-CL may include a switching transistor and a driving transistor for driving the plurality of light emitting elements ED-1, ED-2 and ED-3 of the display element layer DP-ED.

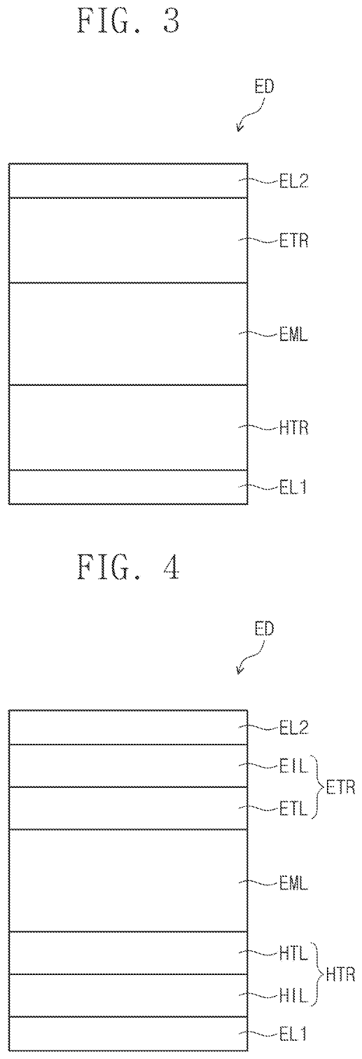

The light emitting elements ED-1, ED-2, and ED-3 may each have a structure of a light emitting element ED according to an embodiment of FIGS. 3 to 6, as described in more detail elsewhere herein. The light emitting elements ED-1, ED-2, and ED-3 may each include a first electrode EL1, a hole transport region HTR, emission layers EML-R, EML-G, and EML-B, an electron transport region ETR, and a second electrode EL2.

FIG. 2 shows an embodiment in which the emission layers EML-R, EML-G, and EML-B of the light emitting elements ED-1, ED-2, and ED-3 are located or disposed in openings OH defined in the pixel defining films PDL, and the hole transport region HTR, the electron transport region ETR, and the second electrode EL2 are provided as a common layer throughout the light emitting elements ED-1, ED-2, and ED-3. However, the embodiment of the present disclosure is not limited thereto, and unlike what is shown in FIG. 2, in an embodiment, the hole transport region HTR and the electron transport region ETR may be provided to be patterned inside the openings OH defined in the pixel defining films PDL. For example, in an embodiment, the hole transport region HTR, the emission layers EML-R, EML-G, and EML-B, and the electron transport region ETR, and/or the like of the light emitting elements ED-1, ED-2, and ED-3 may be patterned and/or provided through an inkjet printing method.

The encapsulation layer TFE may cover the light emitting elements ED-1, ED-2 and ED-3. The encapsulation layer TFE may seal the display element layer DP-ED. The encapsulation layer TFE may be a thin film encapsulation layer. The encapsulation layer TFE may be a single layer or a laminated layer of a plurality of layers. The encapsulation layer TFE includes at least one insulating layer. The encapsulation layer TFE according to an embodiment may include at least one inorganic film (hereinafter, an encapsulation inorganic film). In some embodiments, the encapsulation layer TFE according to an embodiment may include at least one organic film (hereinafter, an encapsulation organic film) and at least one encapsulation inorganic film.

The encapsulation inorganic film protects the display element layer DP-ED from moisture and/or oxygen, and the encapsulation organic film protects the display element layer DP-ED from foreign substances such as dust particles. The encapsulation inorganic film may include silicon nitride, silicon oxy nitride, silicon oxide, titanium oxide, aluminum oxide, and/or the like, but is not limited thereto. The encapsulation organic film may include an acrylic compound, an epoxy-based compound, and/or the like. The encapsulation organic film may include a photopolymerizable organic material, but is not limited thereto.

The encapsulation layer TFE may be located or disposed on the second electrode EL2, and may be located or disposed to fill the openings OH.

Referring to FIGS. 1 and 2, the display device DD may include one or more non-light emitting regions NPXA and light emitting regions PXA-R, PXA-G, and PXA-B. The light emitting regions PXA-R, PXA-G, and PXA-B may each be a region emitting light generated from each of the light emitting elements ED-1, ED-2, and ED-3. The light emitting regions PXA-R, PXA-G, and PXA-B may be spaced from each other when viewed on a plane.

The light emitting regions PXA-R, PXA-G, and PXA-B may each be a region separated by the pixel defining films PDL. The non-light emitting regions NPXA may be regions between neighboring light emitting regions PXA-R, PXA-G, and PXA-B, and may correspond to the pixel defining films PDL. In some embodiments, In the present description, the light emitting regions PXA-R, PXA-G, and PXA-B may each correspond to a pixel. The pixel defining films PDL may separate the light emitting elements ED-1, ED-2 and ED-3. The emission layers EML-R, EML-G, and EML-B of the light emitting elements ED-1, ED-2 and ED-3 may be located or disposed and separated in openings OH defined by the pixel defining films PDL.

The light emitting regions PXA-R, PXA-G, and PXA-B may be divided into a plurality of groups according to the color of light generated from the light emitting elements ED-1, ED-2, and ED-3. In the display device DD of an embodiment shown in FIGS. 1 and 2, three light emitting regions PXA-R, PXA-G, and PXA-B which emit red light, green light, and blue light, are shown as an example. For example, the display device DD of an embodiment may include a red light emitting region PXA-R, a green light emitting region PXA-G, and a blue light emitting region PXA-B, which are distinct from one another.

In the display device DD according to an embodiment, the plurality of light emitting elements ED-1, ED-2, and ED-3 may be configured to emit light having different wavelength ranges. For example, in an embodiment, the display device DD may include a first light emitting element ED-1 emitting red light, a second light emitting element ED-2 emitting green light, and a third light emitting element ED-3 emitting blue light. For example, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, the embodiment of the present disclosure is not limited thereto, and the first to third light emitting elements ED-1, ED-2 and ED-3 may be configured to emit light in substantially the same wavelength range or emit light in at least one different wavelength range. For example, the first to third light emitting elements ED-1, ED-2, and ED-3 all may be configured to emit blue light.

The light emitting regions PXA-R, PXA-G, and PXA-B in the display device DD according to an embodiment may be arranged in the form of a stripe. Referring to FIG. 1, a plurality of red light emitting regions PXA-R may be with each other arranged along a second directional axis DR2, a plurality of green light emitting regions PXA-G may be with each other arranged along the second directional axis DR2, and a plurality of blue light emitting regions PXA-B may be arranged with each other along the second directional axis DR2. In some embodiments, the (one) red light emitting region PXA-R, the (one) green light emitting region PXA-G, and the (one) blue light emitting region PXA-B may be alternately arranged with other in turn along a first directional axis DR1.

FIGS. 1 and 2 show that the light emitting regions PXA-R, PXA-G, and PXA-B are all similar in size, but the embodiment of the present disclosure is not limited thereto, and the light emitting regions PXA-R, PXA-G and PXA-B may be different in size from each other according to wavelength range of emitted light. In some embodiments, the areas of the light emitting regions PXA-R, PXA-G, and PXA-B may refer to regions when viewed on a plane defined by the first directional axis DR1 and the second directional axis DR2 (in a plan view).

In some embodiments, the arrangement of the light emitting regions PXA-R, PXA-G, and PXA-B is not limited to what is shown in FIG. 1, and the order in which the red light emitting region PXA-R, the green light emitting region PXA-G, and the blue light emitting region PXA-B are arranged comes with varied combination according to display quality characteristics required for the display device DD. For example, the light emitting regions PXA-R, PXA-G, and PXA-B may be configured in the PENTILER arrangement form or the Diamond Pixel® arrangement form (PENTILE® and Diamond Pixel® each is a registered trademark owned by Samsung Display Co., Ltd.).

In some embodiments, areas of each of the light emitting regions PXA-R, PXA-G, and PXA-B may be different in size from one another. For example, in an embodiment, the green light emitting region PXA-G may be smaller than the blue light emitting region PXA-B in size, but the embodiment of the present disclosure is not limited thereto.

### Light Emitting Element

FIGS. 3 to 6 are each a cross-sectional view schematically showing a light emitting element according to an embodiment of the present disclosure. For example, a light emitting element ED according to an embodiment may include a first electrode EL1, a hole transport region HTR, an emission layer EML, an electron transport region ETR, and a second electrode EL2.

As illustrated in FIG. 3, the light emitting element ED includes the first electrode EL1, a hole transport region HTR, a light emitting layer EML, an electron transport region ETR, and the second electrode EL2 which are sequentially laminated.

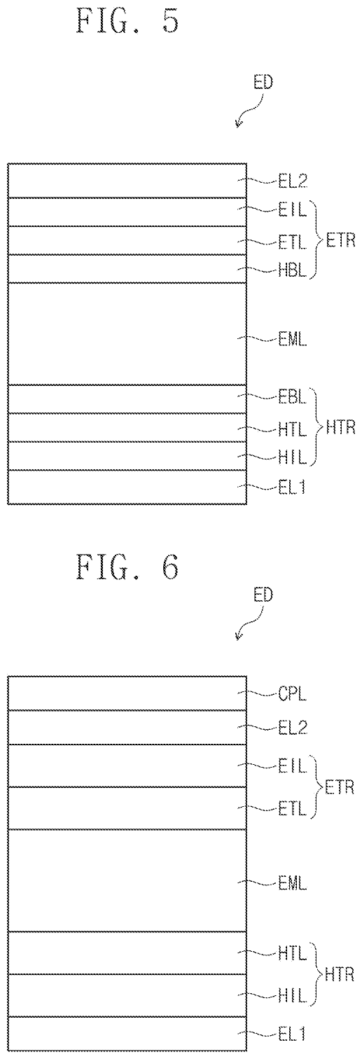

FIG. 4 shows, compared with FIG. 3, a cross-sectional view of a light emitting element ED of an embodiment in which the hole transport region HTR includes a hole injection layer HIL and a hole transport layer HTL, and the electron transport region ETR includes an electron injection layer EIL and an electron transport layer ETL. FIG. 5 shows, compared with FIG. 3, a cross-sectional view of a light emitting element ED according to an embodiment in which the hole transport region HTR includes a hole injection layer HIL, a hole transport layer HTL, and an electron blocking layer EBL, and the electron transport region ETR includes an electron injection layer EIL, an electron transport layer ETL, and a hole blocking layer HBL. FIG. 6 shows, compared with FIG. 4, a cross-sectional view of a light emitting element ED according to an embodiment, in which a capping layer CPL disposed on the second electrode EL2 is included.

The light emitting element ED of an embodiment may include the polycyclic compound of an embodiment in at least one functional layer disposed between a first electrode and a second electrode. At least one functional layer may include a hole transport region HTR, an emission layer EML, and an electron transport region ETR. For example, the hole transport region HTR may include a polycyclic compound of an embodiment.

### Polycyclic Compound

The polycyclic compound of an embodiment may include two carbazole groups and a benzo naphthalo heteroaryl group. The two carbazole groups and benzonaphthalo heteroaryl group may reduce or optimize stacking between individual molecules of the polycyclic compound and enhance or improve the intermolecular interactions thereof, to enable or produce excellent or suitable orientation of the polycyclic compound. The polycyclic compound of an embodiment may have reduced or optimized quenching caused by stacking. The polycyclic compound of an embodiment may have enhanced or improved hole transport ability due to excellent or suitable orientation. Accordingly, the light emitting element ED including the polycyclic compound of an embodiment may exhibit enhanced or improved (e.g., high) power efficiency.

The light emitting element ED according to an embodiment may include a polycyclic compound according to an embodiment. The polycyclic compound according to an embodiment may be represented by Formula 1.

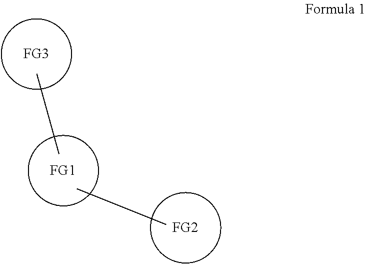

In Formula 1, FG1 may be represented by Formula 1-1.

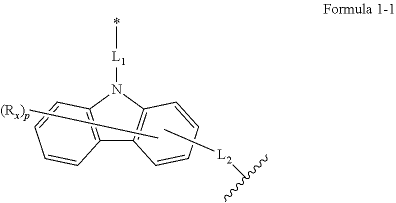

In Formula 1-1, “ [symbol] ” is a portion bonded to FG2, and “ [symbol] ” is a portion bonded to FG3.

L₁ may be a direct linkage or a substituted or unsubstituted arylene group having 6 to 30 ring-forming carbon atoms, When Li is a direct linkage, FG1 may be directly bonded to FG3, and when L₁ is a substituted or unsubstituted arylene group having 6 to 30 ring-forming carbon atoms, FG1 may be bonded to FG3 through L₁.

L₂ may be a direct linkage or a substituted or unsubstituted arylene group having 6 to 30 ring-forming carbon atoms, When L₂ is a direct linkage, FG1 is directly bonded to FG2. When L₂ is a substituted or unsubstituted arylene group having 6 to 30 ring carbon atoms, FG1 may be bonded to FG2 through L₂.

Rₓ may be a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, Rₓ may be a hydrogen atom or an unsubstituted phenyl group.

In Formula 1-1, p may be an integer of 0 to 7 that represents or indicates the number of Rₓ groups to be substituted. In some embodiments, when p is an integer of 2 to 7, a plurality of Rₓ groups may all be the same or at least one may be different from the others.

In Formula 1, FG2 may be represented by Formula 1-2.

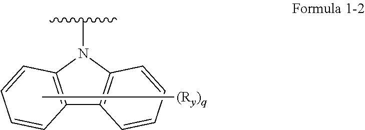

In Formula 1-2, “ [symbol] ” is a portion bonded to FG1.

R_{y} may be a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R_{y} may be a hydrogen atom, a deuterium atom, a substituted or unsubstituted phenyl group, an unsubstituted naphthyl group, an unsubstituted dibenzofuran group, or an unsubstituted dibenzothiophene group.

In Formula 1-2, q may be an integer of 0 to 8 that represents or indicates the number of R_{y} groups to be substituted. In some embodiments, when q is an integer of 2 to 8, a plurality of R_{y} groups may all be the same or at least one may be different from the others.

In Formula 1, FG3 may be represented by any one among Formulas 1-3 to 1-5.

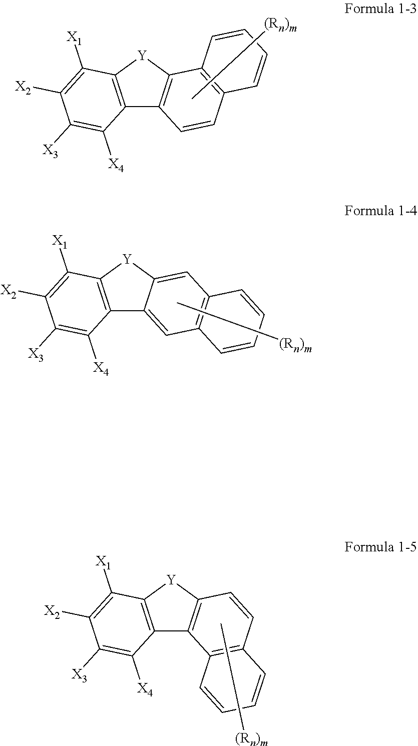

In Formula 1-3 to Formula 1-5, at least (e.g., any) one among X₁ to X₄ may be a portion bonded to FG1 of Formula 1, and the other X₁ to X₄ may each independently be a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, each of X₁ to X₄ that is not bonded to FG1 of Formula 1 may be a hydrogen atom.

In Formula 1-3 to Formula 1-5, Y may be O or S. When Y is O, FG3 may be a benzonaphthalofuran group. The polycyclic compound of an embodiment that includes a benzonaphthalofuran group may have excellent or suitable hole transport ability. In an embodiment, hole transport region HTR, the hole transport layer HTL and/or the light emitting element including the polycyclic compound of the present disclosure having the benzonaphthalofuran group, as described herein, may have excellent or suitable hole transport ability. When Y is S, FG3 may be a benzonaphthalothiophene group. The polycyclic compound of an embodiment that includes a benzonaphthalothiophene group may have excellent or suitable hole transport ability. In an embodiment, hole transport region HTR, the hole transport layer HTL and/or the light emitting element including the polycyclic compound of the present disclosure having the benzonaphthalothiophene group, as described herein, may have excellent or suitable hole transport ability.

In Formula 1-3 to Formula 1-5, m may be an integer of 0 to 6 that represents the number of Rₙ groups included in FG3. Rₙ may be a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In some embodiments, when m is an integer of 2 or greater, a plurality of Rₙ groups may all be the same or at least one may be different from the others.

Formula 1 may be represented by any one among Formulas 2-1 to 2-3. Each of Formulas 2-1 to 2-3 locates (or specifically defines the position) where L₂ is connected to (or substituted on) a carbazole group that represents FG1 in Formula 1.

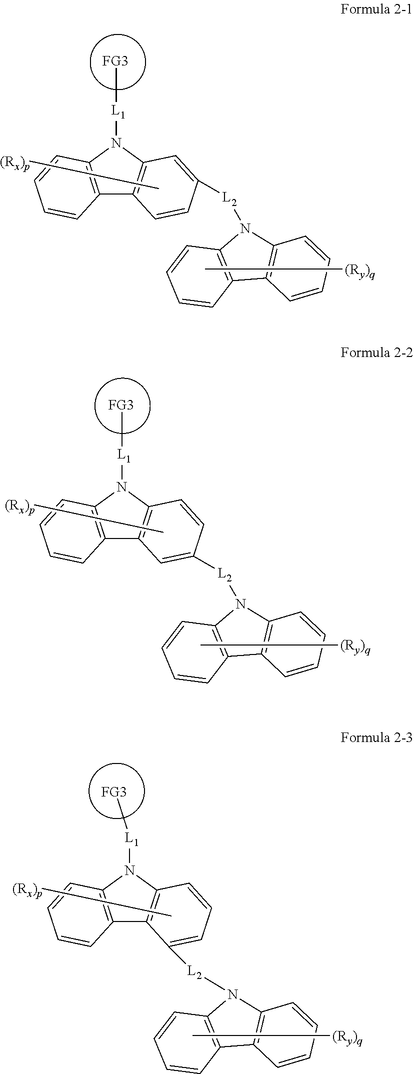

In Formulas 2-1 to 2-3, FG3, L₁, L₂, Rₓ, R_{y}, p, and q may be the same as defined in Formula 1.

In an embodiment, Formula 1 above may be represented by Formula 3-1 or Formula 3-2. Each of Formula 3-1 and Formula 3-2 describes (or specifically defines) L₁ in Formula 1. Formula 3-1 is a case where L₁ in Formula 1 is a direct linkage. Formula 3-2 is a case where L₁ in Formula 1 is a phenylene group.

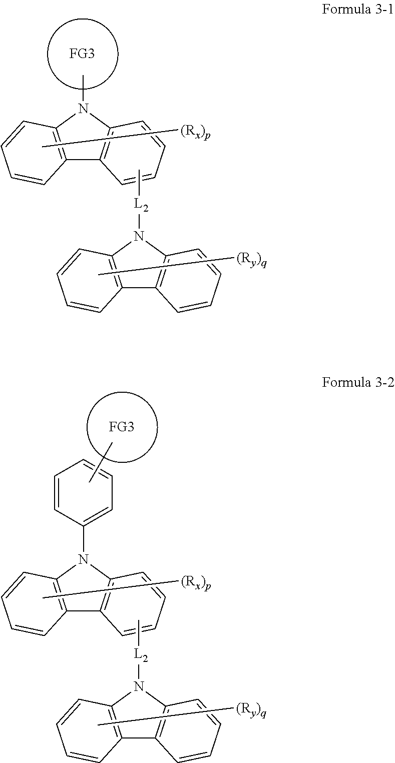

In Formulas 3-1 and 3-2, FG3, L₂, Rₓ, R_{y}, p, and q may be the same as defined in Formula 1 above.

In an embodiment, Formula 3-2 may be represented by Formula 3A or Formula 3B. Formulas 3A and 3B locate (or specifically define positions) where FG3 is connected to (or substituted on) L₁ in Formula 3-2. Formula 3A is a case in which the carbazole group that represents FG1 and FG3 are positioned para with respect to L₁ that is represented as a phenylene group. Formula 3B is a case in which the carbazole group that represents FG1 and FG3 are positioned meta with respect to L₁ that is represented as a phenylene group.

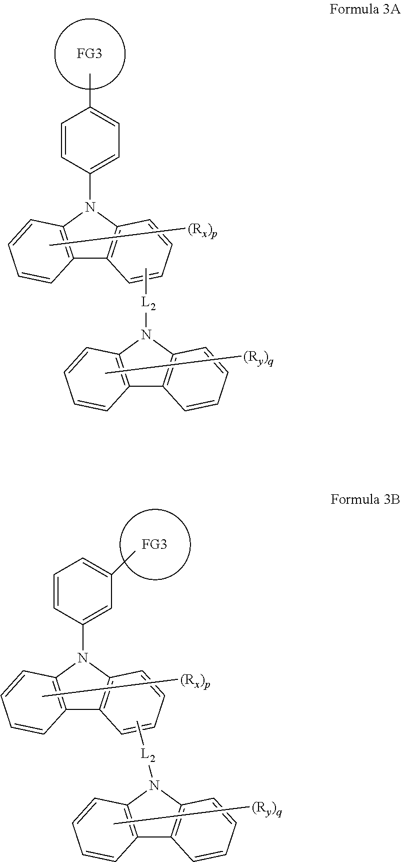

In Formulas 3A and 3B, FG3, L₂, Rₓ, R_{y}, p, and q may be the same as defined in Formula 1.

In an embodiment, Formula 1 may be represented by Formula 4-1 or Formula 4-2. Each of Formula 4-1 and Formula 4-2 describe (or specifically defines) L₂ in Formula 1. Formula 4-1 is a case where L₂ is a direct linkage. Formula 4-2 is a case where L₂ is a phenylene group.

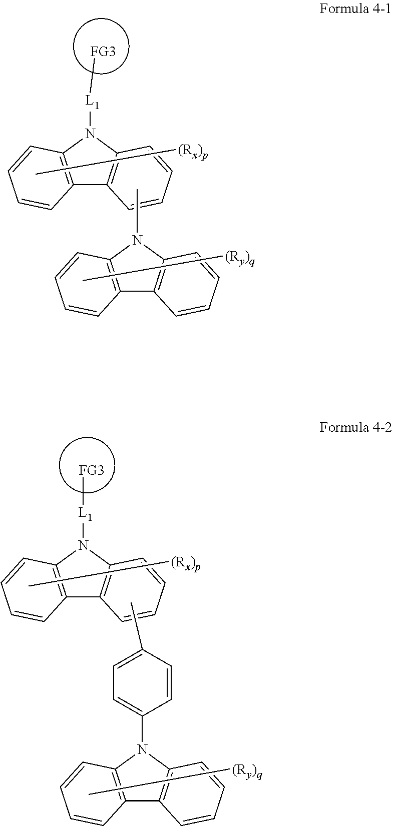

In each of Formulas 4-1 and 4-2, FG3, L₁, Rₓ, R_{y}, p, and q may be the same as defined in Formula 1.

In Formula 1 above, FG1 may be represented by any one selected from among B-1 to B-7.

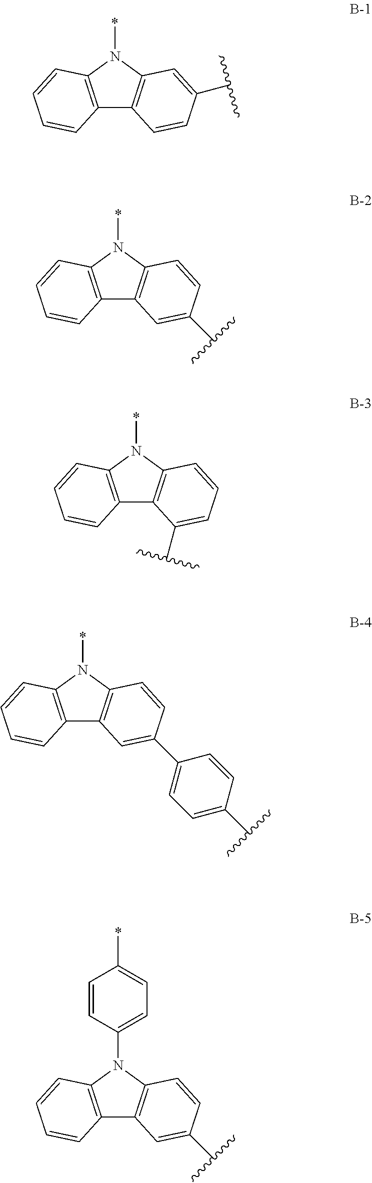 [SMILES: *c1ccc2c3ccccc3n(*)c2c1 ; *c1ccc2c(c1)c1ccccc1n2* ; *c1cccc2c1c1ccccc1n2* ; *c1ccc(-c2ccc3c(c2)c2ccccc2n3*)cc1 ; *c1ccc(-n2c3ccccc3c3cc(*)ccc32)cc1]

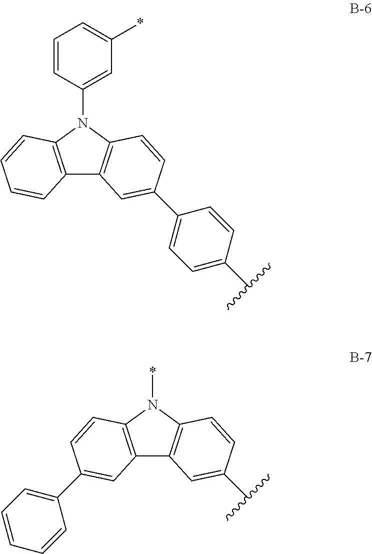 [SMILES: *c1ccc(-c2ccc3c(c2)c2ccccc2n3-c2cccc(*)c2)cc1 ; *c1ccc2c(c1)c1cc(-c3ccccc3)ccc1n2*]

In Formula 1 above, FG2 may be represented by any one selected from among C-1 to C-8.

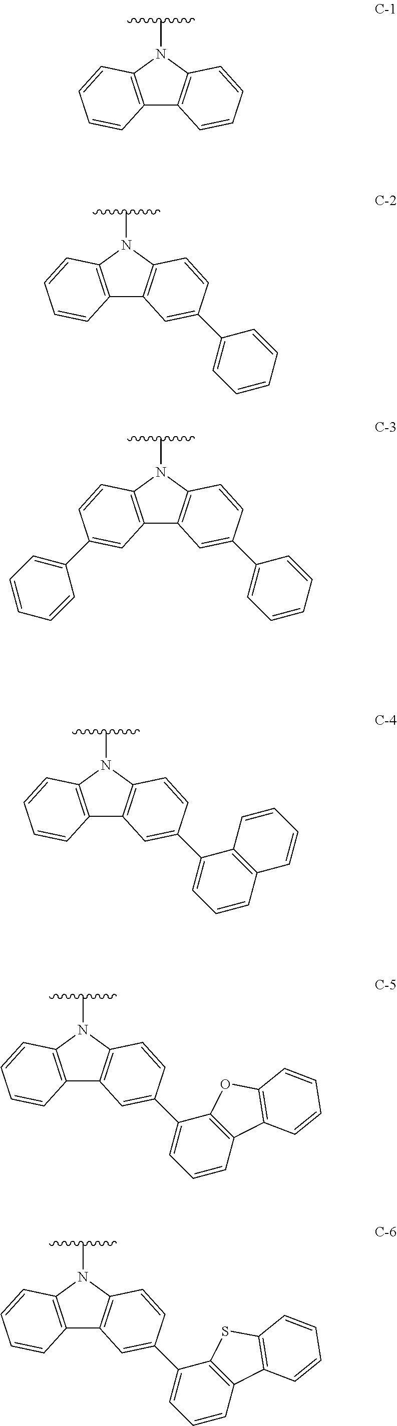 [SMILES: *n1c2ccccc2c2ccccc21 ; *n1c2ccccc2c2cc(-c3ccccc3)ccc21 ; *n1c2ccc(-c3ccccc3)cc2c2cc(-c3ccccc3)ccc21 ; *n1c2ccccc2c2cc(-c3cccc4ccccc34)ccc21 ; *n1c2ccccc2c2cc(-c3cccc4c3oc3ccccc34)ccc21 ; *n1c2ccccc2c2cc(-c3cccc4c3sc3ccccc34)ccc21]

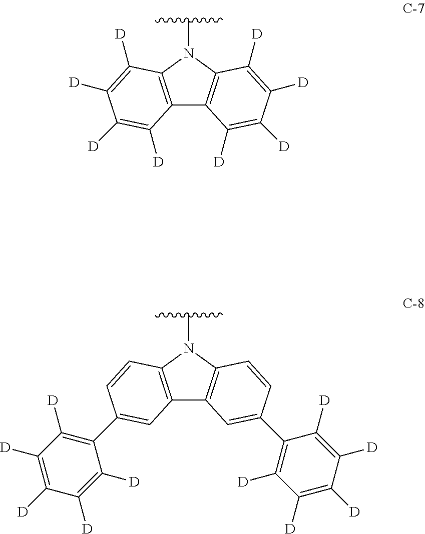 [SMILES: *n1c2c([2H])c([2H])c([2H])c([2H])c2c2c([2H])c([2H])c([2H])c([2H])c21 ; *n1c2ccc(-c3c([2H])c([2H])c([2H])c([2H])c3[2H])cc2c2cc(-c3c([2H])c([2H])c([2H])c([2H])c3[2H])ccc21]

The polycyclic compound represented by Formula 1 may be represented by any one selected from among the compounds shown in Table 1. Referring to Table 1, the “definition of FG3” indicates that FG3 represented by Formula 1 is represented by any one among Formulas 1-3 to 1-5. As described in Formulas 1-3 to 1-5, the “bonding position of FG3 and FG1” indicates which one among X₁ to X₄ in FG3 is bonded to FG1. The “Y” indicates whether Y is O or S in Formulas 1-3 to 1-5. The “FG1” indicates any one among B-1 to B-7 described above. The “FG2” indicates any one among C-1 to C-8 described above.

TABLE 1

| Compound | Definition of | Bonding position of | | | |
|---|---|---|---|---|---|
| No. | FG3 | FG3 and FG1 | Y | FG1 | FG2 |
| AO1 | Formula 1-3 | X₁ | O | B-1 | C-1 |
| AO2 | Formula 1-3 | X₁ | O | B-1 | C-2 |
| AO3 | Formula 1-3 | X₁ | O | B-1 | C-3 |
| AO4 | Formula 1-3 | X₁ | O | B-1 | C-4 |
| AO5 | Formula 1-3 | X₁ | O | B-1 | C-5 |
| AO6 | Formula 1-3 | X₁ | O | B-1 | C-6 |
| AO7 | Formula 1-3 | X₁ | O | B-2 | C-1 |
| AO8 | Formula 1-3 | X₁ | O | B-2 | C-2 |
| AO9 | Formula 1-3 | X₁ | O | B-2 | C-3 |
| AO10 | Formula 1-3 | X₁ | O | B-2 | C-4 |
| AO11 | Formula 1-3 | X₁ | O | B-2 | C-5 |
| AO12 | Formula 1-3 | X₁ | O | B-2 | C-6 |
| AO13 | Formula 1-3 | X₁ | O | B-3 | C-1 |
| AO14 | Formula 1-3 | X₁ | O | B-3 | C-2 |
| AO15 | Formula 1-3 | X₁ | O | B-3 | C-3 |
| AO16 | Formula 1-3 | X₁ | O | B-3 | C-4 |
| AO17 | Formula 1-3 | X₁ | O | B-3 | C-5 |
| AO18 | Formula 1-3 | X₁ | O | B-3 | C-6 |
| AO19 | Formula 1-3 | X₁ | O | B-4 | C-1 |
| AO20 | Formula 1-3 | X₁ | O | B-4 | C-2 |
| AO21 | Formula 1-3 | X₁ | O | B-4 | C-3 |
| AO22 | Formula 1-3 | X₁ | O | B-4 | C-4 |
| AO23 | Formula 1-3 | X₁ | O | B-4 | C-5 |
| AO24 | Formula 1-3 | X₁ | O | B-4 | C-6 |
| AO25 | Formula 1-3 | X₁ | O | B-5 | C-1 |
| AO26 | Formula 1-3 | X₁ | O | B-5 | C-2 |
| AO27 | Formula 1-3 | X₁ | O | B-5 | C-3 |
| AO28 | Formula 1-3 | X₁ | O | B-5 | C-4 |
| AO29 | Formula 1-3 | X₁ | O | B-5 | C-5 |
| AO30 | Formula 1-3 | X₁ | O | B-5 | C-6 |
| AO31 | Formula 1-3 | X₁ | O | B-6 | C-1 |
| AO32 | Formula 1-3 | X₁ | O | B-6 | C-2 |
| AO33 | Formula 1-3 | X₁ | O | B-6 | C-3 |
| AO34 | Formula 1-3 | X₁ | O | B-6 | C-4 |
| AO35 | Formula 1-3 | X₁ | O | B-6 | C-5 |
| AO36 | Formula 1-3 | X₁ | O | B-6 | C-6 |
| AO37 | Formula 1-3 | X₁ | O | B-2 | C-7 |
| AO38 | Formula 1-3 | X₁ | O | B-2 | C-8 |
| AO39 | Formula 1-3 | X₁ | O | B-7 | C-1 |
| BO1 | Formula 1-4 | X₁ | O | B-1 | C-1 |
| BO2 | Formula 1-4 | X₁ | O | B-1 | C-2 |
| BO3 | Formula 1-4 | X₁ | O | B-1 | C-3 |
| BO4 | Formula 1-4 | X₁ | O | B-1 | C-4 |
| BO5 | Formula 1-4 | X₁ | O | B-1 | C-5 |
| BO6 | Formula 1-4 | X₁ | O | B-1 | C-6 |
| BO7 | Formula 1-4 | X₁ | O | B-2 | C-1 |
| BO8 | Formula 1-4 | X₁ | O | B-2 | C-2 |
| BO9 | Formula 1-4 | X₁ | O | B-2 | C-3 |
| BO10 | Formula 1-4 | X₁ | O | B-2 | C-4 |
| BO11 | Formula 1-4 | X₁ | O | B-2 | C-5 |
| BO12 | Formula 1-4 | X₁ | O | B-2 | C-6 |
| BO13 | Formula 1-4 | X₁ | O | B-3 | C-1 |
| BO14 | Formula 1-4 | X₁ | O | B-3 | C-2 |
| BO15 | Formula 1-4 | X₁ | O | B-3 | C-3 |
| BO16 | Formula 1-4 | X₁ | O | B-3 | C-4 |
| BO17 | Formula 1-4 | X₁ | O | B-3 | C-5 |
| BO18 | Formula 1-4 | X₁ | O | B-3 | C-6 |
| BO19 | Formula 1-4 | X₁ | O | B-4 | C-1 |
| BO20 | Formula 1-4 | X₁ | O | B-4 | C-2 |
| BO21 | Formula 1-4 | X₁ | O | B-4 | C-3 |
| BO22 | Formula 1-4 | X₁ | O | B-4 | C-4 |
| BO23 | Formula 1-4 | X₁ | O | B-4 | C-5 |
| BO24 | Formula 1-4 | X₁ | O | B-4 | C-6 |
| BO25 | Formula 1-4 | X₁ | O | B-5 | C-1 |
| BO26 | Formula 1-4 | X₁ | O | B-5 | C-2 |
| BO27 | Formula 1-4 | X₁ | O | B-5 | C-3 |
| BO28 | Formula 1-4 | X₁ | O | B-5 | C-4 |
| BO29 | Formula 1-4 | X₁ | O | B-5 | C-5 |
| BO30 | Formula 1-4 | X₁ | O | B-5 | C-6 |
| BO31 | Formula 1-4 | X₁ | O | B-6 | C-1 |
| BO32 | Formula 1-4 | X₁ | O | B-6 | C-2 |
| BO33 | Formula 1-4 | X₁ | O | B-6 | C-3 |
| BO34 | Formula 1-4 | X₁ | O | B-6 | C-4 |
| BO35 | Formula 1-4 | X₁ | O | B-6 | C-5 |
| BO36 | Formula 1-4 | X₁ | O | B-6 | C-6 |
| BO37 | Formula 1-4 | X₁ | O | B-2 | C-7 |
| BO38 | Formula 1-4 | X₁ | O | B-2 | C-8 |
| CO1 | Formula 1-5 | X₁ | O | B-1 | C-1 |
| CO2 | Formula 1-5 | X₁ | O | B-1 | C-2 |
| CO3 | Formula 1-5 | X₁ | O | B-1 | C-3 |
| CO4 | Formula 1-5 | X₁ | O | B-1 | C-4 |
| CO5 | Formula 1-5 | X₁ | O | B-1 | C-5 |
| CO6 | Formula 1-5 | X₁ | O | B-1 | C-6 |
| CO7 | Formula 1-5 | X₁ | O | B-2 | C-1 |
| CO8 | Formula 1-5 | X₁ | O | B-2 | C-2 |
| CO9 | Formula 1-5 | X₁ | O | B-2 | C-3 |
| CO10 | Formula 1-5 | X₁ | O | B-2 | C-4 |
| CO11 | Formula 1-5 | X₁ | O | B-2 | C-5 |
| CO12 | Formula 1-5 | X₁ | O | B-2 | C-6 |
| CO13 | Formula 1-5 | X₁ | O | B-3 | C-1 |
| CO14 | Formula 1-5 | X₁ | O | B-3 | C-2 |
| CO15 | Formula 1-5 | X₁ | O | B-3 | C-3 |
| CO16 | Formula 1-5 | X₁ | O | B-3 | C-4 |
| CO17 | Formula 1-5 | X₁ | O | B-3 | C-5 |
| CO18 | Formula 1-5 | X₁ | O | B-3 | C-6 |
| CO19 | Formula 1-5 | X₁ | O | B-4 | C-1 |
| CO20 | Formula 1-5 | X₁ | O | B-4 | C-2 |
| CO21 | Formula 1-5 | X₁ | O | B-4 | C-3 |
| CO22 | Formula 1-5 | X₁ | O | B-4 | C-4 |
| CO23 | Formula 1-5 | X₁ | O | B-4 | C-5 |
| CO24 | Formula 1-5 | X₁ | O | B-4 | C-6 |
| CO25 | Formula 1-5 | X₁ | O | B-5 | C-1 |
| CO26 | Formula 1-5 | X₁ | O | B-5 | C-2 |
| CO27 | Formula 1-5 | X₁ | O | B-5 | C-3 |
| CO28 | Formula 1-5 | X₁ | O | B-5 | C-4 |
| CO29 | Formula 1-5 | X₁ | O | B-5 | C-5 |
| CO30 | Formula 1-5 | X₁ | O | B-5 | C-6 |
| CO31 | Formula 1-5 | X₁ | O | B-6 | C-1 |
| CO32 | Formula 1-5 | X₁ | O | B-6 | C-2 |
| CO33 | Formula 1-5 | X₁ | O | B-6 | C-3 |
| CO34 | Formula 1-5 | X₁ | O | B-6 | C-4 |
| CO35 | Formula 1-5 | X₁ | O | B-6 | C-5 |
| CO36 | Formula 1-5 | X₁ | O | B-6 | C-6 |
| CO37 | Formula 1-5 | X₁ | O | B-2 | C-7 |
| CO38 | Formula 1-5 | X₁ | O | B-2 | C-8 |
| DO1 | Formula 1-3 | X₂ | O | B-1 | C-1 |
| DO2 | Formula 1-3 | X₂ | O | B-1 | C-2 |
| DO3 | Formula 1-3 | X₂ | O | B-1 | C-3 |
| DO4 | Formula 1-3 | X₂ | O | B-1 | C-4 |
| DO5 | Formula 1-3 | X₂ | O | B-1 | C-5 |
| DO6 | Formula 1-3 | X₂ | O | B-1 | C-6 |
| DO7 | Formula 1-3 | X₂ | O | B-2 | C-1 |
| DO8 | Formula 1-3 | X₂ | O | B-2 | C-2 |
| DO9 | Formula 1-3 | X₂ | O | B-2 | C-3 |
| DO10 | Formula 1-3 | X₂ | O | B-2 | C-4 |
| DO11 | Formula 1-3 | X₂ | O | B-2 | C-5 |
| DO12 | Formula 1-3 | X₂ | O | B-2 | C-6 |
| DO13 | Formula 1-3 | X₂ | O | B-3 | C-1 |
| DO14 | Formula 1-3 | X₂ | O | B-3 | C-2 |
| DO15 | Formula 1-3 | X₂ | O | B-3 | C-3 |
| DO16 | Formula 1-3 | X₂ | O | B-3 | C-4 |
| DO17 | Formula 1-3 | X₂ | O | B-3 | C-5 |
| DO18 | Formula 1-3 | X₂ | O | B-3 | C-6 |
| DO19 | Formula 1-3 | X₂ | O | B-4 | C-1 |
| DO20 | Formula 1-3 | X₂ | O | B-4 | C-2 |
| DO21 | Formula 1-3 | X₂ | O | B-4 | C-3 |
| DO22 | Formula 1-3 | X₂ | O | B-4 | C-4 |
| DO23 | Formula 1-3 | X₂ | O | B-4 | C-5 |
| DO24 | Formula 1-3 | X₂ | O | B-4 | C-6 |
| DO25 | Formula 1-3 | X₂ | O | B-5 | C-1 |
| DO26 | Formula 1-3 | X₂ | O | B-5 | C-2 |
| DO27 | Formula 1-3 | X₂ | O | B-5 | C-3 |
| DO28 | Formula 1-3 | X₂ | O | B-5 | C-4 |
| DO29 | Formula 1-3 | X₂ | O | B-5 | C-5 |
| DO30 | Formula 1-3 | X₂ | O | B-5 | C-6 |
| DO31 | Formula 1-3 | X₂ | O | B-6 | C-1 |
| DO32 | Formula 1-3 | X₂ | O | B-6 | C-2 |
| DO33 | Formula 1-3 | X₂ | O | B-6 | C-3 |
| DO34 | Formula 1-3 | X₂ | O | B-6 | C-4 |
| DO35 | Formula 1-3 | X₂ | O | B-6 | C-5 |
| DO36 | Formula 1-3 | X₂ | O | B-6 | C-6 |
| DO37 | Formula 1-3 | X₂ | O | B-2 | C-7 |
| DO38 | Formula 1-3 | X₂ | O | B-2 | C-8 |
| EO1 | Formula 1-4 | X₂ | O | B-1 | C-1 |
| EO2 | Formula 1-4 | X₂ | O | B-1 | C-2 |
| EO3 | Formula 1-4 | X₂ | O | B-1 | C-3 |
| EO4 | Formula 1-4 | X₂ | O | B-1 | C-4 |
| EO5 | Formula 1-4 | X₂ | O | B-1 | C-5 |
| EO6 | Formula 1-4 | X₂ | O | B-1 | C-6 |
| EO7 | Formula 1-4 | X₂ | O | B-2 | C-1 |
| EO8 | Formula 1-4 | X₂ | O | B-2 | C-2 |
| EO9 | Formula 1-4 | X₂ | O | B-2 | C-3 |
| EO10 | Formula 1-4 | X₂ | O | B-2 | C-4 |
| EO11 | Formula 1-4 | X₂ | O | B-2 | C-5 |
| EO12 | Formula 1-4 | X₂ | O | B-2 | C-6 |
| EO13 | Formula 1-4 | X₂ | O | B-3 | C-1 |
| EO14 | Formula 1-4 | X₂ | O | B-3 | C-2 |
| EO15 | Formula 1-4 | X₂ | O | B-3 | C-3 |
| EO16 | Formula 1-4 | X₂ | O | B-3 | C-4 |
| EO17 | Formula 1-4 | X₂ | O | B-3 | C-5 |
| EO18 | Formula 1-4 | X₂ | O | B-3 | C-6 |
| EO19 | Formula 1-4 | X₂ | O | B-4 | C-1 |
| EO20 | Formula 1-4 | X₂ | O | B-4 | C-2 |
| EO21 | Formula 1-4 | X₂ | O | B-4 | C-3 |
| EO22 | Formula 1-4 | X₂ | O | B-4 | C-4 |
| EO23 | Formula 1-4 | X₂ | O | B-4 | C-5 |
| EO24 | Formula 1-4 | X₂ | O | B-4 | C-6 |
| EO25 | Formula 1-4 | X₂ | O | B-5 | C-1 |
| EO26 | Formula 1-4 | X₂ | O | B-5 | C-2 |
| EO27 | Formula 1-4 | X₂ | O | B-5 | C-3 |
| EO28 | Formula 1-4 | X₂ | O | B-5 | C-4 |
| EO29 | Formula 1-4 | X₂ | O | B-5 | C-5 |
| EO30 | Formula 1-4 | X₂ | O | B-5 | C-6 |
| EO31 | Formula 1-4 | X₂ | O | B-6 | C-1 |
| EO32 | Formula 1-4 | X₂ | O | B-6 | C-2 |
| EO33 | Formula 1-4 | X₂ | O | B-6 | C-3 |
| EO34 | Formula 1-4 | X₂ | O | B-6 | C-4 |
| EO35 | Formula 1-4 | X₂ | O | B-6 | C-5 |
| EO36 | Formula 1-4 | X₂ | O | B-6 | C-6 |
| EO37 | Formula 1-4 | X₂ | O | B-2 | C-7 |
| EO38 | Formula 1-4 | X₂ | O | B-2 | C-8 |
| FO1 | Formula 1-5 | X₂ | O | B-1 | C-1 |
| FO2 | Formula 1-5 | X₂ | O | B-1 | C-2 |
| FO3 | Formula 1-5 | X₂ | O | B-1 | C-3 |
| FO4 | Formula 1-5 | X₂ | O | B-1 | C-4 |
| FO5 | Formula 1-5 | X₂ | O | B-1 | C-5 |
| FO6 | Formula 1-5 | X₂ | O | B-1 | C-6 |
| FO7 | Formula 1-5 | X₂ | O | B-2 | C-1 |
| FO8 | Formula 1-5 | X₂ | O | B-2 | C-2 |
| FO9 | Formula 1-5 | X₂ | O | B-2 | C-3 |
| FO10 | Formula 1-5 | X₂ | O | B-2 | C-4 |
| FO11 | Formula 1-5 | X₂ | O | B-2 | C-5 |
| FO12 | Formula 1-5 | X₂ | O | B-2 | C-6 |
| FO13 | Formula 1-5 | X₂ | O | B-3 | C-1 |
| FO14 | Formula 1-5 | X₂ | O | B-3 | C-2 |
| FO15 | Formula 1-5 | X₂ | O | B-3 | C-3 |
| FO16 | Formula 1-5 | X₂ | O | B-3 | C-4 |
| FO17 | Formula 1-5 | X₂ | O | B-3 | C-5 |
| FO18 | Formula 1-5 | X₂ | O | B-3 | C-6 |
| FO19 | Formula 1-5 | X₂ | O | B-4 | C-1 |
| FO20 | Formula 1-5 | X₂ | O | B-4 | C-2 |
| FO21 | Formula 1-5 | X₂ | O | B-4 | C-3 |
| FO22 | Formula 1-5 | X₂ | O | B-4 | C-4 |
| FO23 | Formula 1-5 | X₂ | O | B-4 | C-5 |
| FO24 | Formula 1-5 | X₂ | O | B-4 | C-6 |
| FO25 | Formula 1-5 | X₂ | O | B-5 | C-1 |
| FO26 | Formula 1-5 | X₂ | O | B-5 | C-2 |
| FO27 | Formula 1-5 | X₂ | O | B-5 | C-3 |
| FO28 | Formula 1-5 | X₂ | O | B-5 | C-4 |
| FO29 | Formula 1-5 | X₂ | O | B-5 | C-5 |
| FO30 | Formula 1-5 | X₂ | O | B-5 | C-6 |
| FO31 | Formula 1-5 | X₂ | O | B-6 | C-1 |
| FO32 | Formula 1-5 | X₂ | O | B-6 | C-2 |
| FO33 | Formula 1-5 | X₂ | O | B-6 | C-3 |
| FO34 | Formula 1-5 | X₂ | O | B-6 | C-4 |
| FO35 | Formula 1-5 | X₂ | O | B-6 | C-5 |
| FO36 | Formula 1-5 | X₂ | O | B-6 | C-6 |
| FO37 | Formula 1-5 | X₂ | O | B-2 | C-7 |
| FO38 | Formula 1-5 | X₂ | O | B-2 | C-8 |
| GO1 | Formula 1-3 | X₃ | O | B-1 | C-1 |
| GO2 | Formula 1-3 | X₃ | O | B-1 | C-2 |
| GO3 | Formula 1-3 | X₃ | O | B-1 | C-3 |
| GO4 | Formula 1-3 | X₃ | O | B-1 | C-4 |
| GO5 | Formula 1-3 | X₃ | O | B-1 | C-5 |
| GO6 | Formula 1-3 | X₃ | O | B-1 | C-6 |
| GO7 | Formula 1-3 | X₃ | O | B-2 | C-1 |
| GO8 | Formula 1-3 | X₃ | O | B-2 | C-2 |
| GO9 | Formula 1-3 | X₃ | O | B-2 | C-3 |
| GO10 | Formula 1-3 | X₃ | O | B-2 | C-4 |
| GO11 | Formula 1-3 | X₃ | O | B-2 | C-5 |
| GO12 | Formula 1-3 | X₃ | O | B-2 | C-6 |
| GO13 | Formula 1-3 | X₃ | O | B-3 | C-1 |
| GO14 | Formula 1-3 | X₃ | O | B-3 | C-2 |
| GO15 | Formula 1-3 | X₃ | O | B-3 | C-3 |
| GO16 | Formula 1-3 | X₃ | O | B-3 | C-4 |
| GO17 | Formula 1-3 | X₃ | O | B-3 | C-5 |
| GO18 | Formula 1-3 | X₃ | O | B-3 | C-6 |
| GO19 | Formula 1-3 | X₃ | O | B-4 | C-1 |
| GO20 | Formula 1-3 | X₃ | O | B-4 | C-2 |
| GO21 | Formula 1-3 | X₃ | O | B-4 | C-3 |
| GO22 | Formula 1-3 | X₃ | O | B-4 | C-4 |
| GO23 | Formula 1-3 | X₃ | O | B-4 | C-5 |
| GO24 | Formula 1-3 | X₃ | O | B-4 | C-6 |
| GO25 | Formula 1-3 | X₃ | O | B-5 | C-1 |
| GO26 | Formula 1-3 | X₃ | O | B-5 | C-2 |
| GO27 | Formula 1-3 | X₃ | O | B-5 | C-3 |
| GO28 | Formula 1-3 | X₃ | O | B-5 | C-4 |
| GO29 | Formula 1-3 | X₃ | O | B-5 | C-5 |
| GO30 | Formula 1-3 | X₃ | O | B-5 | C-6 |
| GO31 | Formula 1-3 | X₃ | O | B-6 | C-1 |
| GO32 | Formula 1-3 | X₃ | O | B-6 | C-2 |
| GO33 | Formula 1-3 | X₃ | O | B-6 | C-3 |
| GO34 | Formula 1-3 | X₃ | O | B-6 | C-4 |
| GO35 | Formula 1-3 | X₃ | O | B-6 | C-5 |
| GO36 | Formula 1-3 | X₃ | O | B-6 | C-6 |
| GO37 | Formula 1-3 | X₃ | O | B-2 | C-7 |
| GO38 | Formula 1-3 | X₃ | O | B-2 | C-8 |
| HO1 | Formula 1-4 | X₃ | O | B-1 | C-1 |
| HO2 | Formula 1-4 | X₃ | O | B-1 | C-2 |
| HO3 | Formula 1-4 | X₃ | O | B-1 | C-3 |
| HO4 | Formula 1-4 | X₃ | O | B-1 | C-4 |
| HO5 | Formula 1-4 | X₃ | O | B-1 | C-5 |
| HO6 | Formula 1-4 | X₃ | O | B-1 | C-6 |
| HO7 | Formula 1-4 | X₃ | O | B-2 | C-1 |
| HO8 | Formula 1-4 | X₃ | O | B-2 | C-2 |
| HO9 | Formula 1-4 | X₃ | O | B-2 | C-3 |
| HO10 | Formula 1-4 | X₃ | O | B-2 | C-4 |
| HO11 | Formula 1-4 | X₃ | O | B-2 | C-5 |
| HO12 | Formula 1-4 | X₃ | O | B-2 | C-6 |
| HO13 | Formula 1-4 | X₃ | O | B-3 | C-1 |
| HO14 | Formula 1-4 | X₃ | O | B-3 | C-2 |
| HO15 | Formula 1-4 | X₃ | O | B-3 | C-3 |
| HO16 | Formula 1-4 | X₃ | O | B-3 | C-4 |
| HO17 | Formula 1-4 | X₃ | O | B-3 | C-5 |
| HO18 | Formula 1-4 | X₃ | O | B-3 | C-6 |
| HO19 | Formula 1-4 | X₃ | O | B-4 | C-1 |
| HO20 | Formula 1-4 | X₃ | O | B-4 | C-2 |
| HO21 | Formula 1-4 | X₃ | O | B-4 | C-3 |
| HO22 | Formula 1-4 | X₃ | O | B-4 | C-4 |
| HO23 | Formula 1-4 | X₃ | O | B-4 | C-5 |
| HO24 | Formula 1-4 | X₃ | O | B-4 | C-6 |
| HO25 | Formula 1-4 | X₃ | O | B-5 | C-1 |
| HO26 | Formula 1-4 | X₃ | O | B-5 | C-2 |
| HO27 | Formula 1-4 | X₃ | O | B-5 | C-3 |
| HO28 | Formula 1-4 | X₃ | O | B-5 | C-4 |
| HO29 | Formula 1-4 | X₃ | O | B-5 | C-5 |
| HO30 | Formula 1-4 | X₃ | O | B-5 | C-6 |
| HO31 | Formula 1-4 | X₃ | O | B-6 | C-1 |
| HO32 | Formula 1-4 | X₃ | O | B-6 | C-2 |
| HO33 | Formula 1-4 | X₃ | O | B-6 | C-3 |
| HO34 | Formula 1-4 | X₃ | O | B-6 | C-4 |
| HO35 | Formula 1-4 | X₃ | O | B-6 | C-5 |
| HO36 | Formula 1-4 | X₃ | O | B-6 | C-6 |
| HO37 | Formula 1-4 | X₃ | O | B-2 | C-7 |
| HO38 | Formula 1-4 | X₃ | O | B-2 | C-8 |
| IO1 | Formula 1-5 | X₃ | O | B-1 | C-1 |
| IO2 | Formula 1-5 | X₃ | O | B-1 | C-2 |
| IO3 | Formula 1-5 | X₃ | O | B-1 | C-3 |
| IO4 | Formula 1-5 | X₃ | O | B-1 | C-4 |
| IO5 | Formula 1-5 | X₃ | O | B-1 | C-5 |
| IO6 | Formula 1-5 | X₃ | O | B-1 | C-6 |
| IO7 | Formula 1-5 | X₃ | O | B-2 | C-1 |
| IO8 | Formula 1-5 | X₃ | O | B-2 | C-2 |
| IO9 | Formula 1-5 | X₃ | O | B-2 | C-3 |
| IO10 | Formula 1-5 | X₃ | O | B-2 | C-4 |
| IO11 | Formula 1-5 | X₃ | O | B-2 | C-5 |
| IO12 | Formula 1-5 | X₃ | O | B-2 | C-6 |
| IO13 | Formula 1-5 | X₃ | O | B-3 | C-1 |
| IO14 | Formula 1-5 | X₃ | O | B-3 | C-2 |
| IO15 | Formula 1-5 | X₃ | O | B-3 | C-3 |
| IO16 | Formula 1-5 | X₃ | O | B-3 | C-4 |
| IO17 | Formula 1-5 | X₃ | O | B-3 | C-5 |
| IO18 | Formula 1-5 | X₃ | O | B-3 | C-6 |
| IO19 | Formula 1-5 | X₃ | O | B-4 | C-1 |
| IO20 | Formula 1-5 | X₃ | O | B-4 | C-2 |
| IO21 | Formula 1-5 | X₃ | O | B-4 | C-3 |
| IO22 | Formula 1-5 | X₃ | O | B-4 | C-4 |
| IO23 | Formula 1-5 | X₃ | O | B-4 | C-5 |
| IO24 | Formula 1-5 | X₃ | O | B-4 | C-6 |
| IO25 | Formula 1-5 | X₃ | O | B-5 | C-1 |
| IO26 | Formula 1-5 | X₃ | O | B-5 | C-2 |
| IO27 | Formula 1-5 | X₃ | O | B-5 | C-3 |
| IO28 | Formula 1-5 | X₃ | O | B-5 | C-4 |
| IO29 | Formula 1-5 | X₃ | O | B-5 | C-5 |
| IO30 | Formula 1-5 | X₃ | O | B-5 | C-6 |
| IO31 | Formula 1-5 | X₃ | O | B-6 | C-1 |
| IO32 | Formula 1-5 | X₃ | O | B-6 | C-2 |
| IO33 | Formula 1-5 | X₃ | O | B-6 | C-3 |
| IO34 | Formula 1-5 | X₃ | O | B-6 | C-4 |
| IO35 | Formula 1-5 | X₃ | O | B-6 | C-5 |
| IO36 | Formula 1-5 | X₃ | O | B-6 | C-6 |
| IO37 | Formula 1-5 | X₃ | O | B-2 | C-7 |
| IO38 | Formula 1-5 | X₃ | O | B-2 | C-8 |
| AS1 | Formula 1-3 | X₁ | S | B-1 | C-1 |
| AS2 | Formula 1-3 | X₁ | S | B-1 | C-2 |
| AS3 | Formula 1-3 | X₁ | S | B-1 | C-3 |
| AS4 | Formula 1-3 | X₁ | S | B-1 | C-4 |
| AS5 | Formula 1-3 | X₁ | S | B-1 | C-5 |
| AS6 | Formula 1-3 | X₁ | S | B-1 | C-6 |
| AS7 | Formula 1-3 | X₁ | S | B-2 | C-1 |
| AS8 | Formula 1-3 | X₁ | S | B-2 | C-2 |
| AS9 | Formula 1-3 | X₁ | S | B-2 | C-3 |
| AS10 | Formula 1-3 | X₁ | S | B-2 | C-4 |
| AS11 | Formula 1-3 | X₁ | S | B-2 | C-5 |
| AS12 | Formula 1-3 | X₁ | S | B-2 | C-6 |
| AS13 | Formula 1-3 | X₁ | S | B-3 | C-1 |
| AS14 | Formula 1-3 | X₁ | S | B-3 | C-2 |
| AS15 | Formula 1-3 | X₁ | S | B-3 | C-3 |
| AS16 | Formula 1-3 | X₁ | S | B-3 | C-4 |
| AS17 | Formula 1-3 | X₁ | S | B-3 | C-5 |
| AS18 | Formula 1-3 | X₁ | S | B-3 | C-6 |
| AS19 | Formula 1-3 | X₁ | S | B-4 | C-1 |
| AS20 | Formula 1-3 | X₁ | S | B-4 | C-2 |
| AS21 | Formula 1-3 | X₁ | S | B-4 | C-3 |
| AS22 | Formula 1-3 | X₁ | S | B-4 | C-4 |
| AS23 | Formula 1-3 | X₁ | S | B-4 | C-5 |
| AS24 | Formula 1-3 | X₁ | S | B-4 | C-6 |
| AS25 | Formula 1-3 | X₁ | S | B-5 | C-1 |
| AS26 | Formula 1-3 | X₁ | S | B-5 | C-2 |
| AS27 | Formula 1-3 | X₁ | S | B-5 | C-3 |
| AS28 | Formula 1-3 | X₁ | S | B-5 | C-4 |
| AS29 | Formula 1-3 | X₁ | S | B-5 | C-5 |
| AS30 | Formula 1-3 | X₁ | S | B-5 | C-6 |
| AS31 | Formula 1-3 | X₁ | S | B-6 | C-1 |
| AS32 | Formula 1-3 | X₁ | S | B-6 | C-2 |
| AS33 | Formula 1-3 | X₁ | S | B-6 | C-3 |
| AS34 | Formula 1-3 | X₁ | S | B-6 | C-4 |
| AS35 | Formula 1-3 | X₁ | S | B-6 | C-5 |
| AS36 | Formula 1-3 | X₁ | S | B-6 | C-6 |
| AS37 | Formula 1-3 | X₁ | S | B-2 | C-7 |
| AS38 | Formula 1-3 | X₁ | S | B-2 | C-8 |
| BS1 | Formula 1-4 | X₁ | S | B-1 | C-1 |
| BS2 | Formula 1-4 | X₁ | S | B-1 | C-2 |
| BS3 | Formula 1-4 | X₁ | S | B-1 | C-3 |
| BS4 | Formula 1-4 | X₁ | S | B-1 | C-4 |
| BS5 | Formula 1-4 | X₁ | S | B-1 | C-5 |
| BS6 | Formula 1-4 | X₁ | S | B-1 | C-6 |
| BS7 | Formula 1-4 | X₁ | S | B-2 | C-1 |
| BS8 | Formula 1-4 | X₁ | S | B-2 | C-2 |
| BS9 | Formula 1-4 | X₁ | S | B-2 | C-3 |
| BS10 | Formula 1-4 | X₁ | S | B-2 | C-4 |
| BS11 | Formula 1-4 | X₁ | S | B-2 | C-5 |
| BS12 | Formula 1-4 | X₁ | S | B-2 | C-6 |
| BS13 | Formula 1-4 | X₁ | S | B-3 | C-1 |
| BS14 | Formula 1-4 | X₁ | S | B-3 | C-2 |
| BS15 | Formula 1-4 | X₁ | S | B-3 | C-3 |
| BS16 | Formula 1-4 | X₁ | S | B-3 | C-4 |
| BS17 | Formula 1-4 | X₁ | S | B-3 | C-5 |
| BS18 | Formula 1-4 | X₁ | S | B-3 | C-6 |
| BS19 | Formula 1-4 | X₁ | S | B-4 | C-1 |
| BS20 | Formula 1-4 | X₁ | S | B-4 | C-2 |
| BS21 | Formula 1-4 | X₁ | S | B-4 | C-3 |
| BS22 | Formula 1-4 | X₁ | S | B-4 | C-4 |
| BS23 | Formula 1-4 | X₁ | S | B-4 | C-5 |
| BS24 | Formula 1-4 | X₁ | S | B-4 | C-6 |
| BS25 | Formula 1-4 | X₁ | S | B-5 | C-1 |
| BS26 | Formula 1-4 | X₁ | S | B-5 | C-2 |
| BS27 | Formula 1-4 | X₁ | S | B-5 | C-3 |
| BS28 | Formula 1-4 | X₁ | S | B-5 | C-4 |
| BS29 | Formula 1-4 | X₁ | S | B-5 | C-5 |
| BS30 | Formula 1-4 | X₁ | S | B-5 | C-6 |
| BS31 | Formula 1-4 | X₁ | S | B-6 | C-1 |
| BS32 | Formula 1-4 | X₁ | S | B-6 | C-2 |
| BS33 | Formula 1-4 | X₁ | S | B-6 | C-3 |
| BS34 | Formula 1-4 | X₁ | S | B-6 | C-4 |
| BS35 | Formula 1-4 | X₁ | S | B-6 | C-5 |
| BS36 | Formula 1-4 | X₁ | S | B-6 | C-6 |
| BS37 | Formula 1-4 | X₁ | S | B-2 | C-7 |
| BS38 | Formula 1-4 | X₁ | S | B-2 | C-8 |
| CS1 | Formula 1-5 | X₁ | S | B-1 | C-1 |
| CS2 | Formula 1-5 | X₁ | S | B-1 | C-2 |
| CS3 | Formula 1-5 | X₁ | S | B-1 | C-3 |
| CS4 | Formula 1-5 | X₁ | S | B-1 | C-4 |
| CS5 | Formula 1-5 | X₁ | S | B-1 | C-5 |
| CS6 | Formula 1-5 | X₁ | S | B-1 | C-6 |
| CS7 | Formula 1-5 | X₁ | S | B-2 | C-1 |
| CS8 | Formula 1-5 | X₁ | S | B-2 | C-2 |
| CS9 | Formula 1-5 | X₁ | S | B-2 | C-3 |
| CS10 | Formula 1-5 | X₁ | S | B-2 | C-4 |
| CS11 | Formula 1-5 | X₁ | S | B-2 | C-5 |
| CS12 | Formula 1-5 | X₁ | S | B-2 | C-6 |
| CS13 | Formula 1-5 | X₁ | S | B-3 | C-1 |
| CS14 | Formula 1-5 | X₁ | S | B-3 | C-2 |
| CS15 | Formula 1-5 | X₁ | S | B-3 | C-3 |
| CS16 | Formula 1-5 | X₁ | S | B-3 | C-4 |
| CS17 | Formula 1-5 | X₁ | S | B-3 | C-5 |
| CS18 | Formula 1-5 | X₁ | S | B-3 | C-6 |
| CS19 | Formula 1-5 | X₁ | S | B-4 | C-1 |
| CS20 | Formula 1-5 | X₁ | S | B-4 | C-2 |
| CS21 | Formula 1-5 | X₁ | S | B-4 | C-3 |
| CS22 | Formula 1-5 | X₁ | S | B-4 | C-4 |
| CS23 | Formula 1-5 | X₁ | S | B-4 | C-5 |
| CS24 | Formula 1-5 | X₁ | S | B-4 | C-6 |
| CS25 | Formula 1-5 | X₁ | S | B-5 | C-1 |
| CS26 | Formula 1-5 | X₁ | S | B-5 | C-2 |
| CS27 | Formula 1-5 | X₁ | S | B-5 | C-3 |
| CS28 | Formula 1-5 | X₁ | S | B-5 | C-4 |
| CS29 | Formula 1-5 | X₁ | S | B-5 | C-5 |
| CS30 | Formula 1-5 | X₁ | S | B-5 | C-6 |
| CS31 | Formula 1-5 | X₁ | S | B-6 | C-1 |
| CS32 | Formula 1-5 | X₁ | S | B-6 | C-2 |
| CS33 | Formula 1-5 | X₁ | S | B-6 | C-3 |
| CS34 | Formula 1-5 | X₁ | S | B-6 | C-4 |
| CS35 | Formula 1-5 | X₁ | S | B-6 | C-5 |
| CS36 | Formula 1-5 | X₁ | S | B-6 | C-6 |
| CS37 | Formula 1-5 | X₁ | S | B-2 | C-7 |
| CS38 | Formula 1-5 | X₁ | S | B-2 | C-8 |
| DS1 | Formula 1-3 | X₂ | S | B-1 | C-1 |
| DS2 | Formula 1-3 | X₂ | S | B-1 | C-2 |
| DS3 | Formula 1-3 | X₂ | S | B-1 | C-3 |
| DS4 | Formula 1-3 | X₂ | S | B-1 | C-4 |
| DS5 | Formula 1-3 | X₂ | S | B-1 | C-5 |
| DS6 | Formula 1-3 | X₂ | S | B-1 | C-6 |
| DS7 | Formula 1-3 | X₂ | S | B-2 | C-1 |
| DS8 | Formula 1-3 | X₂ | S | B-2 | C-2 |
| DS9 | Formula 1-3 | X₂ | S | B-2 | C-3 |
| DS10 | Formula 1-3 | X₂ | S | B-2 | C-4 |
| DS11 | Formula 1-3 | X₂ | S | B-2 | C-5 |
| DS12 | Formula 1-3 | X₂ | S | B-2 | C-6 |
| DS13 | Formula 1-3 | X₂ | S | B-3 | C-1 |
| DS14 | Formula 1-3 | X₂ | S | B-3 | C-2 |
| DS15 | Formula 1-3 | X₂ | S | B-3 | C-3 |
| DS16 | Formula 1-3 | X₂ | S | B-3 | C-4 |
| DS17 | Formula 1-3 | X₂ | S | B-3 | C-5 |
| DS18 | Formula 1-3 | X₂ | S | B-3 | C-6 |
| DS19 | Formula 1-3 | X₂ | S | B-4 | C-1 |
| DS20 | Formula 1-3 | X₂ | S | B-4 | C-2 |
| DS21 | Formula 1-3 | X₂ | S | B-4 | C-3 |
| DS22 | Formula 1-3 | X₂ | S | B-4 | C-4 |
| DS23 | Formula 1-3 | X₂ | S | B-4 | C-5 |
| DS24 | Formula 1-3 | X₂ | S | B-4 | C-6 |
| DS25 | Formula 1-3 | X₂ | S | B-5 | C-1 |
| DS26 | Formula 1-3 | X₂ | S | B-5 | C-2 |
| DS27 | Formula 1-3 | X₂ | S | B-5 | C-3 |
| DS28 | Formula 1-3 | X₂ | S | B-5 | C-4 |
| DS29 | Formula 1-3 | X₂ | S | B-5 | C-5 |
| DS30 | Formula 1-3 | X₂ | S | B-5 | C-6 |
| DS31 | Formula 1-3 | X₂ | S | B-6 | C-1 |
| DS32 | Formula 1-3 | X₂ | S | B-6 | C-2 |
| DS33 | Formula 1-3 | X₂ | S | B-6 | C-3 |
| DS34 | Formula 1-3 | X₂ | S | B-6 | C-4 |
| DS35 | Formula 1-3 | X₂ | S | B-6 | C-5 |
| DS36 | Formula 1-3 | X₂ | S | B-6 | C-6 |
| DS37 | Formula 1-3 | X₂ | S | B-2 | C-7 |
| DS38 | Formula 1-3 | X₂ | S | B-2 | C-8 |
| ES1 | Formula 1-4 | X₂ | S | B-1 | C-1 |
| ES2 | Formula 1-4 | X₂ | S | B-1 | C-2 |
| ES3 | Formula 1-4 | X₂ | S | B-1 | C-3 |
| ES4 | Formula 1-4 | X₂ | S | B-1 | C-4 |
| ES5 | Formula 1-4 | X₂ | S | B-1 | C-5 |
| ES6 | Formula 1-4 | X₂ | S | B-1 | C-6 |
| ES7 | Formula 1-4 | X₂ | S | B-2 | C-1 |
| ES8 | Formula 1-4 | X₂ | S | B-2 | C-2 |
| ES9 | Formula 1-4 | X₂ | S | B-2 | C-3 |
| ES10 | Formula 1-4 | X₂ | S | B-2 | C-4 |
| ES11 | Formula 1-4 | X₂ | S | B-2 | C-5 |
| ES12 | Formula 1-4 | X₂ | S | B-2 | C-6 |
| ES13 | Formula 1-4 | X₂ | S | B-3 | C-1 |
| ES14 | Formula 1-4 | X₂ | S | B-3 | C-2 |
| ES15 | Formula 1-4 | X₂ | S | B-3 | C-3 |
| ES16 | Formula 1-4 | X₂ | S | B-3 | C-4 |
| ES17 | Formula 1-4 | X₂ | S | B-3 | C-5 |
| ES18 | Formula 1-4 | X₂ | S | B-3 | C-6 |
| ES19 | Formula 1-4 | X₂ | S | B-4 | C-1 |
| ES20 | Formula 1-4 | X₂ | S | B-4 | C-2 |
| ES21 | Formula 1-4 | X₂ | S | B-4 | C-3 |
| ES22 | Formula 1-4 | X₂ | S | B-4 | C-4 |
| ES23 | Formula 1-4 | X₂ | S | B-4 | C-5 |
| ES24 | Formula 1-4 | X₂ | S | B-4 | C-6 |
| ES25 | Formula 1-4 | X₂ | S | B-5 | C-1 |
| ES26 | Formula 1-4 | X₂ | S | B-5 | C-2 |
| ES27 | Formula 1-4 | X₂ | S | B-5 | C-3 |
| ES28 | Formula 1-4 | X₂ | S | B-5 | C-4 |
| ES29 | Formula 1-4 | X₂ | S | B-5 | C-5 |
| ES30 | Formula 1-4 | X₂ | S | B-5 | C-6 |
| ES31 | Formula 1-4 | X₂ | S | B-6 | C-1 |
| ES32 | Formula 1-4 | X₂ | S | B-6 | C-2 |
| ES33 | Formula 1-4 | X₂ | S | B-6 | C-3 |
| ES34 | Formula 1-4 | X₂ | S | B-6 | C-4 |
| ES35 | Formula 1-4 | X₂ | S | B-6 | C-5 |
| ES36 | Formula 1-4 | X₂ | S | B-6 | C-6 |
| ES37 | Formula 1-4 | X₂ | S | B-2 | C-7 |
| ES38 | Formula 1-4 | X₂ | S | B-2 | C-8 |
| FS1 | Formula 1-5 | X₂ | S | B-1 | C-1 |
| FS2 | Formula 1-5 | X₂ | S | B-1 | C-2 |
| FS3 | Formula 1-5 | X₂ | S | B-1 | C-3 |
| FS4 | Formula 1-5 | X₂ | S | B-1 | C-4 |
| FS5 | Formula 1-5 | X₂ | S | B-1 | C-5 |
| FS6 | Formula 1-5 | X₂ | S | B-1 | C-6 |
| FS7 | Formula 1-5 | X₂ | S | B-2 | C-1 |
| FS8 | Formula 1-5 | X₂ | S | B-2 | C-2 |
| FS9 | Formula 1-5 | X₂ | S | B-2 | C-3 |
| FS10 | Formula 1-5 | X₂ | S | B-2 | C-4 |
| FS11 | Formula 1-5 | X₂ | S | B-2 | C-5 |
| FS12 | Formula 1-5 | X₂ | S | B-2 | C-6 |
| FS13 | Formula 1-5 | X₂ | S | B-3 | C-1 |
| FS14 | Formula 1-5 | X₂ | S | B-3 | C-2 |
| FS15 | Formula 1-5 | X₂ | S | B-3 | C-3 |
| FS16 | Formula 1-5 | X₂ | S | B-3 | C-4 |
| FS17 | Formula 1-5 | X₂ | S | B-3 | C-5 |
| FS18 | Formula 1-5 | X₂ | S | B-3 | C-6 |
| FS19 | Formula 1-5 | X₂ | S | B-4 | C-1 |
| FS20 | Formula 1-5 | X₂ | S | B-4 | C-2 |
| FS21 | Formula 1-5 | X₂ | S | B-4 | C-3 |
| FS22 | Formula 1-5 | X₂ | S | B-4 | C-4 |
| FS23 | Formula 1-5 | X₂ | S | B-4 | C-5 |
| FS24 | Formula 1-5 | X₂ | S | B-4 | C-6 |
| FS25 | Formula 1-5 | X₂ | S | B-5 | C-1 |
| FS26 | Formula 1-5 | X₂ | S | B-5 | C-2 |
| FS27 | Formula 1-5 | X₂ | S | B-5 | C-3 |
| FS28 | Formula 1-5 | X₂ | S | B-5 | C-4 |
| FS29 | Formula 1-5 | X₂ | S | B-5 | C-5 |
| FS30 | Formula 1-5 | X₂ | S | B-5 | C-6 |
| FS31 | Formula 1-5 | X₂ | S | B-6 | C-1 |
| FS32 | Formula 1-5 | X₂ | S | B-6 | C-2 |
| FS33 | Formula 1-5 | X₂ | S | B-6 | C-3 |
| FS34 | Formula 1-5 | X₂ | S | B-6 | C-4 |
| FS35 | Formula 1-5 | X₂ | S | B-6 | C-5 |
| FS36 | Formula 1-5 | X₂ | S | B-6 | C-6 |
| FS37 | Formula 1-5 | X₂ | S | B-2 | C-7 |
| FS38 | Formula 1-5 | X₂ | S | B-2 | C-8 |
| GS1 | Formula 1-3 | X₃ | S | B-1 | C-1 |
| GS2 | Formula 1-3 | X₃ | S | B-1 | C-2 |
| GS3 | Formula 1-3 | X₃ | S | B-1 | C-3 |
| GS4 | Formula 1-3 | X₃ | S | B-1 | C-4 |
| GS5 | Formula 1-3 | X₃ | S | B-1 | C-5 |
| GS6 | Formula 1-3 | X₃ | S | B-1 | C-6 |
| GS7 | Formula 1-3 | X₃ | S | B-2 | C-1 |
| GS8 | Formula 1-3 | X₃ | S | B-2 | C-2 |
| GS9 | Formula 1-3 | X₃ | S | B-2 | C-3 |
| GS10 | Formula 1-3 | X₃ | S | B-2 | C-4 |
| GS11 | Formula 1-3 | X₃ | S | B-2 | C-5 |
| GS12 | Formula 1-3 | X₃ | S | B-2 | C-6 |
| GS13 | Formula 1-3 | X₃ | S | B-3 | C-1 |
| GS14 | Formula 1-3 | X₃ | S | B-3 | C-2 |
| GS15 | Formula 1-3 | X₃ | S | B-3 | C-3 |
| GS16 | Formula 1-3 | X₃ | S | B-3 | C-4 |
| GS17 | Formula 1-3 | X₃ | S | B-3 | C-5 |
| GS18 | Formula 1-3 | X₃ | S | B-3 | C-6 |
| GS19 | Formula 1-3 | X₃ | S | B-4 | C-1 |
| GS20 | Formula 1-3 | X₃ | S | B-4 | C-2 |
| GS21 | Formula 1-3 | X₃ | S | B-4 | C-3 |
| GS22 | Formula 1-3 | X₃ | S | B-4 | C-4 |
| GS23 | Formula 1-3 | X₃ | S | B-4 | C-5 |
| GS24 | Formula 1-3 | X₃ | S | B-4 | C-6 |
| GS25 | Formula 1-3 | X₃ | S | B-5 | C-1 |
| GS26 | Formula 1-3 | X₃ | S | B-5 | C-2 |
| GS27 | Formula 1-3 | X₃ | S | B-5 | C-3 |
| GS28 | Formula 1-3 | X₃ | S | B-5 | C-4 |
| GS29 | Formula 1-3 | X₃ | S | B-5 | C-5 |
| GS30 | Formula 1-3 | X₃ | S | B-5 | C-6 |
| GS31 | Formula 1-3 | X₃ | S | B-6 | C-1 |
| GS32 | Formula 1-3 | X₃ | S | B-6 | C-2 |
| GS33 | Formula 1-3 | X₃ | S | B-6 | C-3 |
| GS34 | Formula 1-3 | X₃ | S | B-6 | C-4 |
| GS35 | Formula 1-3 | X₃ | S | B-6 | C-5 |
| GS36 | Formula 1-3 | X₃ | S | B-6 | C-6 |
| GS37 | Formula 1-3 | X₃ | S | B-2 | C-7 |
| GS38 | Formula 1-3 | X₃ | S | B-2 | C-8 |
| HS1 | Formula 1-4 | X₃ | S | B-1 | C-1 |
| HS2 | Formula 1-4 | X₃ | S | B-1 | C-2 |
| HS3 | Formula 1-4 | X₃ | S | B-1 | C-3 |
| HS4 | Formula 1-4 | X₃ | S | B-1 | C-4 |
| HS5 | Formula 1-4 | X₃ | S | B-1 | C-5 |
| HS6 | Formula 1-4 | X₃ | S | B-1 | C-6 |
| HS7 | Formula 1-4 | X₃ | S | B-2 | C-1 |
| HS8 | Formula 1-4 | X₃ | S | B-2 | C-2 |
| HS9 | Formula 1-4 | X₃ | S | B-2 | C-3 |
| HS10 | Formula 1-4 | X₃ | S | B-2 | C-4 |
| HS11 | Formula 1-4 | X₃ | S | B-2 | C-5 |
| HS12 | Formula 1-4 | X₃ | S | B-2 | C-6 |
| HS13 | Formula 1-4 | X₃ | S | B-3 | C-1 |
| HS14 | Formula 1-4 | X₃ | S | B-3 | C-2 |
| HS15 | Formula 1-4 | X₃ | S | B-3 | C-3 |
| HS16 | Formula 1-4 | X₃ | S | B-3 | C-4 |
| HS17 | Formula 1-4 | X₃ | S | B-3 | C-5 |
| HS18 | Formula 1-4 | X₃ | S | B-3 | C-6 |
| HS19 | Formula 1-4 | X₃ | S | B-4 | C-1 |
| HS20 | Formula 1-4 | X₃ | S | B-4 | C-2 |
| HS21 | Formula 1-4 | X₃ | S | B-4 | C-3 |
| HS22 | Formula 1-4 | X₃ | S | B-4 | C-4 |
| HS23 | Formula 1-4 | X₃ | S | B-4 | C-5 |
| HS24 | Formula 1-4 | X₃ | S | B-4 | C-6 |
| HS25 | Formula 1-4 | X₃ | S | B-5 | C-1 |
| HS26 | Formula 1-4 | X₃ | S | B-5 | C-2 |
| HS27 | Formula 1-4 | X₃ | S | B-5 | C-3 |
| HS28 | Formula 1-4 | X₃ | S | B-5 | C-4 |
| HS29 | Formula 1-4 | X₃ | S | B-5 | C-5 |
| HS30 | Formula 1-4 | X₃ | S | B-5 | C-6 |
| HS31 | Formula 1-4 | X₃ | S | B-6 | C-1 |
| HS32 | Formula 1-4 | X₃ | S | B-6 | C-2 |
| HS33 | Formula 1-4 | X₃ | S | B-6 | C-3 |
| HS34 | Formula 1-4 | X₃ | S | B-6 | C-4 |
| HS35 | Formula 1-4 | X₃ | S | B-6 | C-5 |
| HS36 | Formula 1-4 | X₃ | S | B-6 | C-6 |
| HS37 | Formula 1-4 | X₃ | S | B-2 | C-7 |
| HS38 | Formula 1-4 | X₃ | S | B-2 | C-8 |
| IS1 | Formula 1-5 | X₃ | S | B-1 | C-1 |
| IS2 | Formula 1-5 | X₃ | S | B-1 | C-2 |
| IS3 | Formula 1-5 | X₃ | S | B-1 | C-3 |
| IS4 | Formula 1-5 | X₃ | S | B-1 | C-4 |
| IS5 | Formula 1-5 | X₃ | S | B-1 | C-5 |
| IS6 | Formula 1-5 | X₃ | S | B-1 | C-6 |
| IS7 | Formula 1-5 | X₃ | S | B-2 | C-1 |
| IS8 | Formula 1-5 | X₃ | S | B-2 | C-2 |
| IS9 | Formula 1-5 | X₃ | S | B-2 | C-3 |
| IS10 | Formula 1-5 | X₃ | S | B-2 | C-4 |
| IS11 | Formula 1-5 | X₃ | S | B-2 | C-5 |
| IS12 | Formula 1-5 | X₃ | S | B-2 | C-6 |
| IS13 | Formula 1-5 | X₃ | S | B-3 | C-1 |
| IS14 | Formula 1-5 | X₃ | S | B-3 | C-2 |
| IS15 | Formula 1-5 | X₃ | S | B-3 | C-3 |
| IS16 | Formula 1-5 | X₃ | S | B-3 | C-4 |
| IS17 | Formula 1-5 | X₃ | S | B-3 | C-5 |
| IS18 | Formula 1-5 | X₃ | S | B-3 | C-6 |
| IS19 | Formula 1-5 | X₃ | S | B-4 | C-1 |
| IS20 | Formula 1-5 | X₃ | S | B-4 | C-2 |
| IS21 | Formula 1-5 | X₃ | S | B-4 | C-3 |
| IS22 | Formula 1-5 | X₃ | S | B-4 | C-4 |
| IS23 | Formula 1-5 | X₃ | S | B-4 | C-5 |
| IS24 | Formula 1-5 | X₃ | S | B-4 | C-6 |
| IS25 | Formula 1-5 | X₃ | S | B-5 | C-1 |
| IS26 | Formula 1-5 | X₃ | S | B-5 | C-2 |
| IS27 | Formula 1-5 | X₃ | S | B-5 | C-3 |
| IS28 | Formula 1-5 | X₃ | S | B-5 | C-4 |
| IS29 | Formula 1-5 | X₃ | S | B-5 | C-5 |
| IS30 | Formula 1-5 | X₃ | S | B-5 | C-6 |
| IS31 | Formula 1-5 | X₃ | S | B-6 | C-1 |
| IS32 | Formula 1-5 | X₃ | S | B-6 | C-2 |
| IS33 | Formula 1-5 | X₃ | S | B-6 | C-3 |
| IS34 | Formula 1-5 | X₃ | S | B-6 | C-4 |
| IS35 | Formula 1-5 | X₃ | S | B-6 | C-5 |
| IS36 | Formula 1-5 | X₃ | S | B-6 | C-6 |
| IS37 | Formula 1-5 | X₃ | S | B-2 | C-7 |
| IS38 | Formula 1-5 | X₃ | S | B-2 | C-8 |
| JS1 | Formula 1-3 | X₄ | S | B-1 | C-1 |
| JS2 | Formula 1-3 | X₄ | S | B-1 | C-2 |
| JS3 | Formula 1-3 | X₄ | S | B-1 | C-3 |
| JS4 | Formula 1-3 | X₄ | S | B-1 | C-4 |
| JS5 | Formula 1-3 | X₄ | S | B-1 | C-5 |
| JS6 | Formula 1-3 | X₄ | S | B-1 | C-6 |
| JS7 | Formula 1-3 | X₄ | S | B-2 | C-1 |
| JS8 | Formula 1-3 | X₄ | S | B-2 | C-2 |
| JS9 | Formula 1-3 | X₄ | S | B-2 | C-3 |
| JS10 | Formula 1-3 | X₄ | S | B-2 | C-4 |
| JS11 | Formula 1-3 | X₄ | S | B-2 | C-5 |
| JS12 | Formula 1-3 | X₄ | S | B-2 | C-6 |
| JS13 | Formula 1-3 | X₄ | S | B-3 | C-1 |
| JS14 | Formula 1-3 | X₄ | S | B-3 | C-2 |
| JS15 | Formula 1-3 | X₄ | S | B-3 | C-3 |
| JS16 | Formula 1-3 | X₄ | S | B-3 | C-4 |
| JS17 | Formula 1-3 | X₄ | S | B-3 | C-5 |
| JS18 | Formula 1-3 | X₄ | S | B-3 | C-6 |
| JS19 | Formula 1-3 | X₄ | S | B-4 | C-1 |
| JS20 | Formula 1-3 | X₄ | S | B-4 | C-2 |
| JS21 | Formula 1-3 | X₄ | S | B-4 | C-3 |
| JS22 | Formula 1-3 | X₄ | S | B-4 | C-4 |
| JS23 | Formula 1-3 | X₄ | S | B-4 | C-5 |
| JS24 | Formula 1-3 | X₄ | S | B-4 | C-6 |
| JS25 | Formula 1-3 | X₄ | S | B-5 | C-1 |
| JS26 | Formula 1-3 | X₄ | S | B-5 | C-2 |
| JS27 | Formula 1-3 | X₄ | S | B-5 | C-3 |
| JS28 | Formula 1-3 | X₄ | S | B-5 | C-4 |
| JS29 | Formula 1-3 | X₄ | S | B-5 | C-5 |
| JS30 | Formula 1-3 | X₄ | S | B-5 | C-6 |
| JS31 | Formula 1-3 | X₄ | S | B-6 | C-1 |
| JS32 | Formula 1-3 | X₄ | S | B-6 | C-2 |
| JS33 | Formula 1-3 | X₄ | S | B-6 | C-3 |
| JS34 | Formula 1-3 | X₄ | S | B-6 | C-4 |
| JS35 | Formula 1-3 | X₄ | S | B-6 | C-5 |
| JS36 | Formula 1-3 | X₄ | S | B-6 | C-6 |
| JS37 | Formula 1-3 | X₄ | S | B-2 | C-7 |
| JS38 | Formula 1-3 | X₄ | S | B-2 | C-8 |
| KS1 | Formula 1-4 | X₄ | S | B-1 | C-1 |
| KS2 | Formula 1-4 | X₄ | S | B-1 | C-2 |
| KS3 | Formula 1-4 | X₄ | S | B-1 | C-3 |
| KS4 | Formula 1-4 | X₄ | S | B-1 | C-4 |
| KS5 | Formula 1-4 | X₄ | S | B-1 | C-5 |
| KS6 | Formula 1-4 | X₄ | S | B-1 | C-6 |
| KS7 | Formula 1-4 | X₄ | S | B-2 | C-1 |
| KS8 | Formula 1-4 | X₄ | S | B-2 | C-2 |
| KS9 | Formula 1-4 | X₄ | S | B-2 | C-3 |
| KS10 | Formula 1-4 | X₄ | S | B-2 | C-4 |
| KS11 | Formula 1-4 | X₄ | S | B-2 | C-5 |
| KS12 | Formula 1-4 | X₄ | S | B-2 | C-6 |
| KS13 | Formula 1-4 | X₄ | S | B-3 | C-1 |
| KS14 | Formula 1-4 | X₄ | S | B-3 | C-2 |
| KS15 | Formula 1-4 | X₄ | S | B-3 | C-3 |
| KS16 | Formula 1-4 | X₄ | S | B-3 | C-4 |
| KS17 | Formula 1-4 | X₄ | S | B-3 | C-5 |
| KS18 | Formula 1-4 | X₄ | S | B-3 | C-6 |
| KS19 | Formula 1-4 | X₄ | S | B-4 | C-1 |
| KS20 | Formula 1-4 | X₄ | S | B-4 | C-2 |
| KS21 | Formula 1-4 | X₄ | S | B-4 | C-3 |
| KS22 | Formula 1-4 | X₄ | S | B-4 | C-4 |
| KS23 | Formula 1-4 | X₄ | S | B-4 | C-5 |
| KS24 | Formula 1-4 | X₄ | S | B-4 | C-6 |
| KS25 | Formula 1-4 | X₄ | S | B-5 | C-1 |
| KS26 | Formula 1-4 | X₄ | S | B-5 | C-2 |
| KS27 | Formula 1-4 | X₄ | S | B-5 | C-3 |
| KS28 | Formula 1-4 | X₄ | S | B-5 | C-4 |
| KS29 | Formula 1-4 | X₄ | S | B-5 | C-5 |
| KS30 | Formula 1-4 | X₄ | S | B-5 | C-6 |
| KS31 | Formula 1-4 | X₄ | S | B-6 | C-1 |
| KS32 | Formula 1-4 | X₄ | S | B-6 | C-2 |
| KS33 | Formula 1-4 | X₄ | S | B-6 | C-3 |
| KS34 | Formula 1-4 | X₄ | S | B-6 | C-4 |
| KS35 | Formula 1-4 | X₄ | S | B-6 | C-5 |
| KS36 | Formula 1-4 | X₄ | S | B-6 | C-6 |
| KS37 | Formula 1-4 | X₄ | S | B-2 | C-7 |
| KS38 | Formula 1-4 | X₄ | S | B-2 | C-8 |
| LS1 | Formula 1-5 | X₄ | S | B-1 | C-1 |
| LS2 | Formula 1-5 | X₄ | S | B-1 | C-2 |
| LS3 | Formula 1-5 | X₄ | S | B-1 | C-3 |
| LS4 | Formula 1-5 | X₄ | S | B-1 | C-4 |
| LS5 | Formula 1-5 | X₄ | S | B-1 | C-5 |
| LS6 | Formula 1-5 | X₄ | S | B-1 | C-6 |
| LS7 | Formula 1-5 | X₄ | S | B-2 | C-1 |
| LS8 | Formula 1-5 | X₄ | S | B-2 | C-2 |
| LS9 | Formula 1-5 | X₄ | S | B-2 | C-3 |
| LS10 | Formula 1-5 | X₄ | S | B-2 | C-4 |
| LS11 | Formula 1-5 | X₄ | S | B-2 | C-5 |
| LS12 | Formula 1-5 | X₄ | S | B-2 | C-6 |
| LS13 | Formula 1-5 | X₄ | S | B-3 | C-1 |
| LS14 | Formula 1-5 | X₄ | S | B-3 | C-2 |
| LS15 | Formula 1-5 | X₄ | S | B-3 | C-3 |
| LS16 | Formula 1-5 | X₄ | S | B-3 | C-4 |
| LS17 | Formula 1-5 | X₄ | S | B-3 | C-5 |
| LS18 | Formula 1-5 | X₄ | S | B-3 | C-6 |
| LS19 | Formula 1-5 | X₄ | S | B-4 | C-1 |
| LS20 | Formula 1-5 | X₄ | S | B-4 | C-2 |
| LS21 | Formula 1-5 | X₄ | S | B-4 | C-3 |
| LS22 | Formula 1-5 | X₄ | S | B-4 | C-4 |
| LS23 | Formula 1-5 | X₄ | S | B-4 | C-5 |
| LS24 | Formula 1-5 | X₄ | S | B-4 | C-6 |
| LS25 | Formula 1-5 | X₄ | S | B-5 | C-1 |
| LS26 | Formula 1-5 | X₄ | S | B-5 | C-2 |
| LS27 | Formula 1-5 | X₄ | S | B-5 | C-3 |
| LS28 | Formula 1-5 | X₄ | S | B-5 | C-4 |
| LS29 | Formula 1-5 | X₄ | S | B-5 | C-5 |
| LS30 | Formula 1-5 | X₄ | S | B-5 | C-6 |
| LS31 | Formula 1-5 | X₄ | S | B-6 | C-1 |
| LS32 | Formula 1-5 | X₄ | S | B-6 | C-2 |
| LS33 | Formula 1-5 | X₄ | S | B-6 | C-3 |
| LS34 | Formula 1-5 | X₄ | S | B-6 | C-4 |
| LS35 | Formula 1-5 | X₄ | S | B-6 | C-5 |
| LS36 | Formula 1-5 | X₄ | S | B-6 | C-6 |
| LS37 | Formula 1-5 | X₄ | S | B-2 | C-7 |
| LS38 | Formula 1-5 | X₄ | S | B-2 | C-8 |

The polycyclic compound of an embodiment includes two carbazole groups and one benzo naphthalo heteroaryl group. Accordingly, the polycyclic compound of an embodiment may have enhanced or improved hole transport ability. A light emitting element of an embodiment includes the polycyclic compound of an embodiment in a hole transport region, and may thus exhibit excellent or suitable power efficiency. The polycyclic compound selected from among the compounds shown in Table 1 may have enhanced or improved hole transport ability. A light emitting element that includes the polycyclic compound selected from among the compounds shown in Table 1 in a hole transport region may exhibit excellent or suitable power efficiency.

Returning to FIGS. 3 to 6, the hole transport region HTR is located or provided on the first electrode EL1. The hole transport region HTR may have, for example, a thickness of about 50 angstrom (Å) to about 15000 Å.

The hole transport region HTR may include at least one of a hole injection layer HIL, a hole transport layer HTL, a buffer layer, a light emitting auxiliary layer, or an electron blocking layer EBL. At least one of the hole injection layer HIL, the hole transport layer HTL, and/or the electron blocking layer EBL may include the polycyclic compound of an embodiment. For example, the hole transport layer HTL may include at least one polycyclic compound of an embodiment.

The hole transport region HTR may have a single layer formed of a single material, a single layer formed of a plurality of different materials, or a multilayer structure having a plurality of layers formed of a plurality of different materials.

For example, the hole transport region HTR may have a single-layer structure formed of the hole injection layer HIL or the hole transport layer HTL, or a single-layer structure formed of a hole injection material or a hole transport material. For example, the hole transport region HTR may have a single-layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order from the first electrode EL1, but the embodiment of the present disclosure is not limited thereto.

The hole transport region HTR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The hole transport region HTR may further include one or more compounds as described herein. For example, the hole transport region HTR may include a compound represented by Formula H-1.

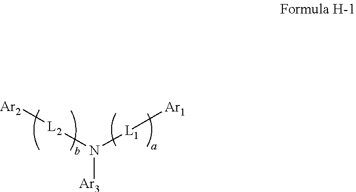

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. In Formula H-1, a and b may each independently be an integer of 0 to 10. In some embodiments, when a or b is an integer of 2 or greater, a plurality of L₁ groups and L₂ groups may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar₃ may be a substituted or unsubstituted aryl group having 6 to 30 ring-forming carbon atoms.

A compound represented by Formula H-1 may be a monoamine compound. In some embodiments, the compound represented by Formula H-1 may be a diamine compound in which at least one of Ar₁ to Ar₃ includes an amine group as a substituent. In some embodiments, the compound represented by Formula H-1 may be a carbazole-based compound including a substituted or unsubstituted carbazole group in at least one of Ar₁ or Ar₂ or a substituted or unsubstituted fluorene-based group in at least one of Ar₁ or Ar₂.

The compound represented by Formula H-1 may be represented by any one selected from among compounds from Compound Group H. However, the compounds listed in Compound Group H are presented as an example, and the compound represented by Formula H-1 is not limited to the those listed in Compound Group H.

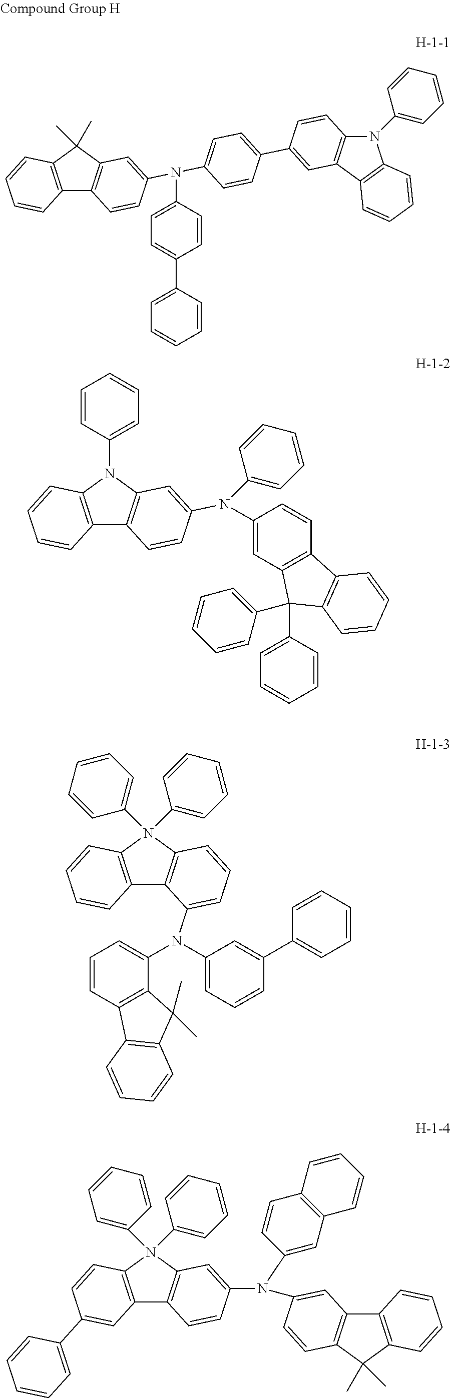

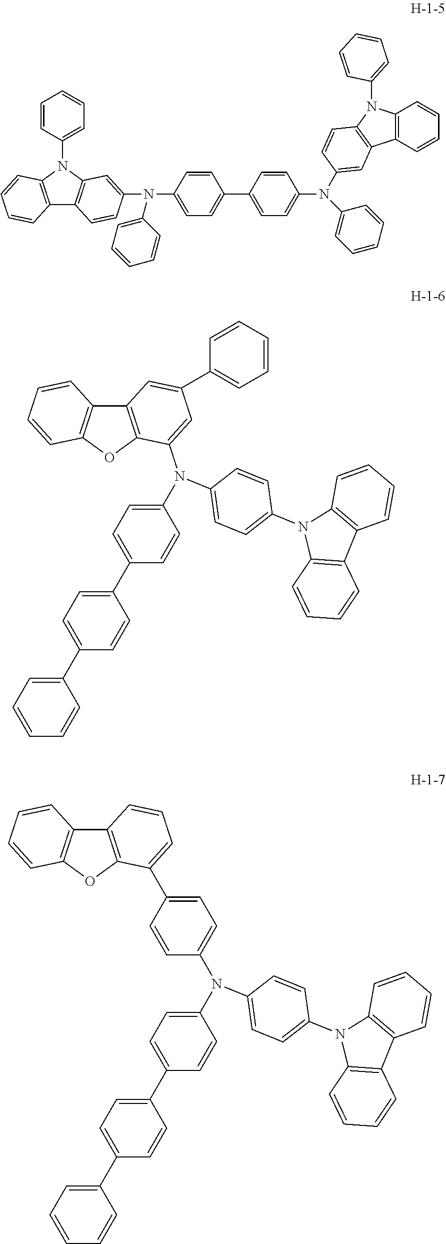 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

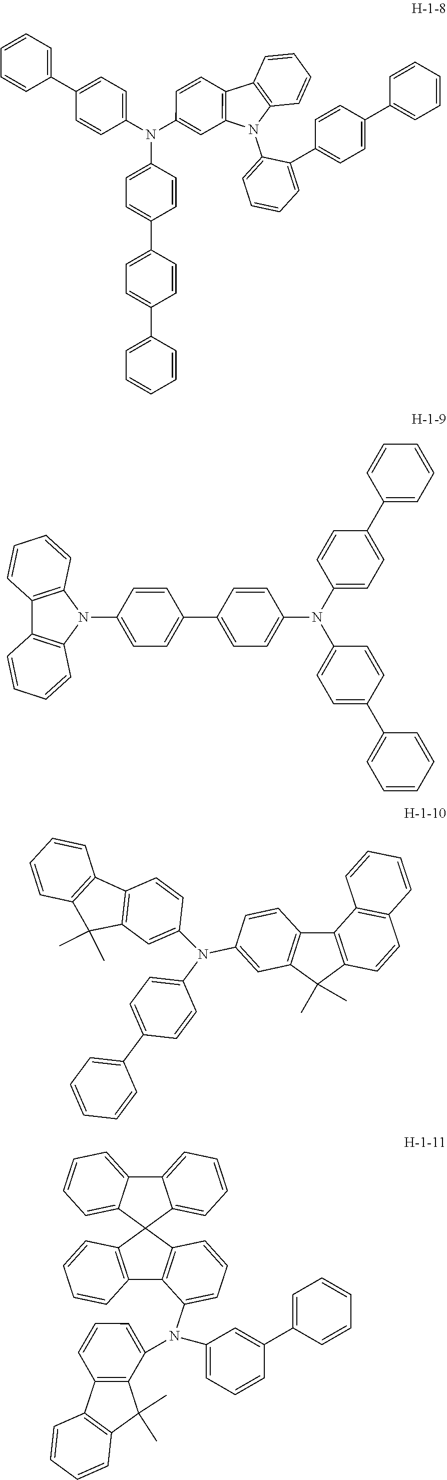

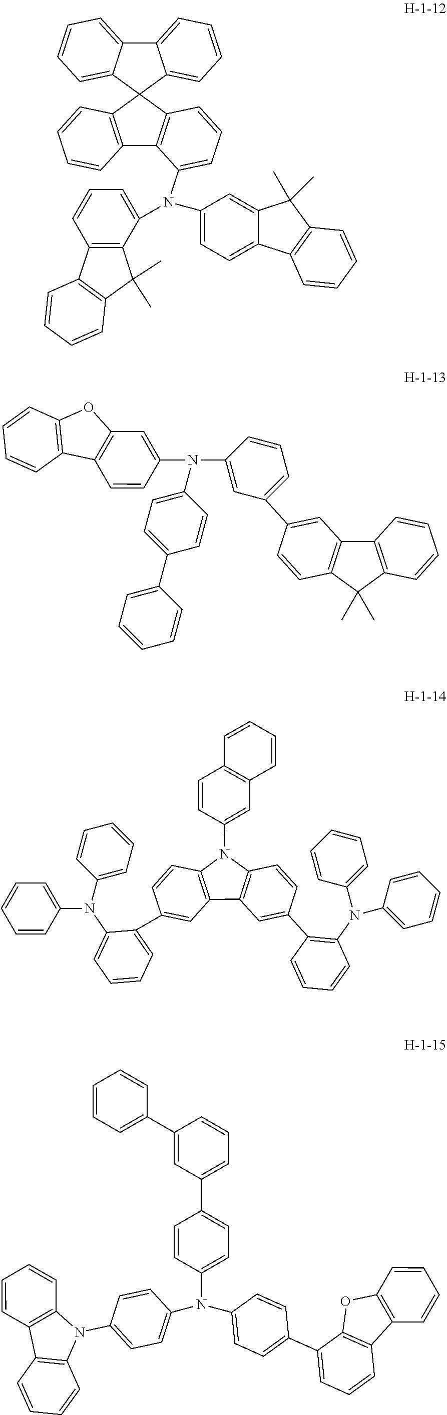 [SMILES: CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1]

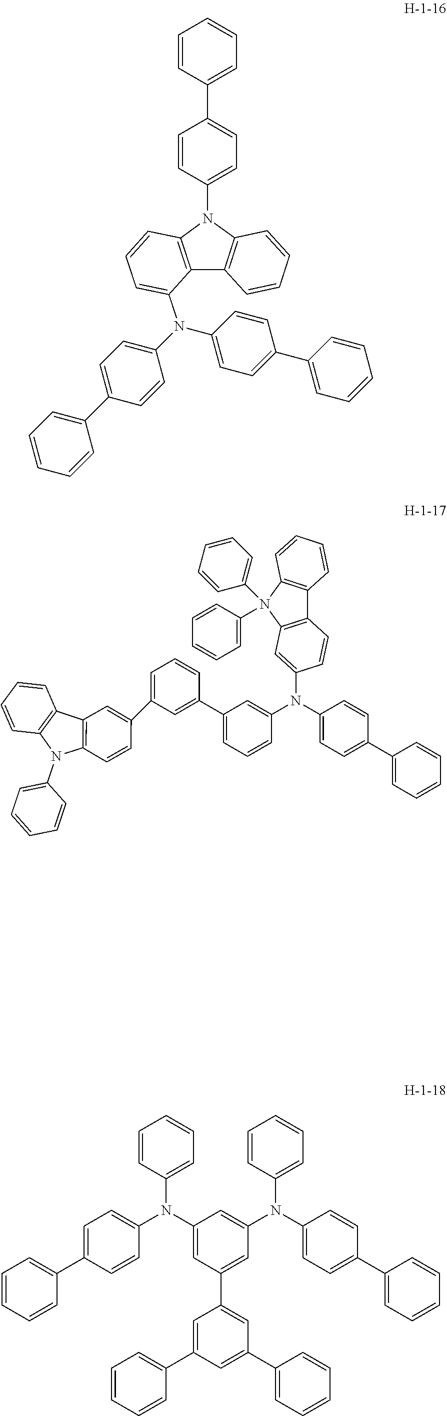

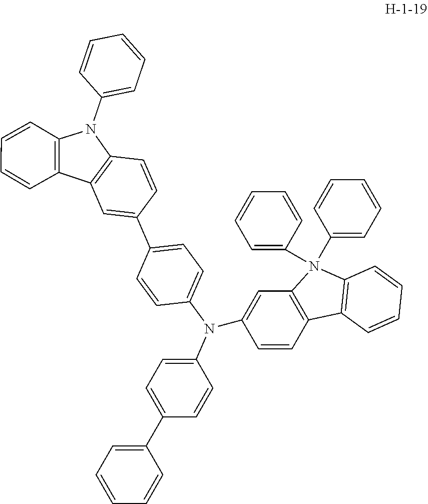

The hole transport region HTR may include a phthalocyanine compound such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′4″-Tris(N,N-diphenylamino)triphenylamine (TDATA), 4,4′,4″-tris[N-(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonicacid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium tetrakis(pentafluorophenyl)borate, dipyrazino[2,3-f: 2′,3′-h]quinoxaline-2,3,6,7,10,11-hexacarbonitrile (HATCN), and/or the like.

The hole transport region HTR may include carbazole-based derivatives such as N-phenyl carbazole and polyvinyl carbazole, fluorene-based derivatives, N,N′-bis(3-methylphenyl)-N,N′-diphenyl-[1,1′-biphenyl]-4,4′-diamine (TPD), triphenylamine-based derivatives such as 4,4′,4″-tris(N-carbazolyl)triphenylamine (TCTA), N,N′-di(naphthalene-I-yl)-N,N′-diphenyl-benzidine (NPB), 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 1,3-bis(N-carbazolyl)benzene (mCP), and/or the like.

In some embodiments, the hole transport region HTR may include 9-(4-tert-Butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include one or more or the compounds of the hole transport region, as described herein, in at least one of the hole injection layer HIL, the hole transport layer HTL, or the electron blocking layer EBL. The hole transport region HTR may have a thickness of about 100 Å to about 10000 Å, for example, about 100 Å to about 5000 Å. When the hole transport region HTR includes the hole injection layer HIL, the hole injection layer HIL may have a thickness of, for example, about 30 Å to about 1000 Å. When the hole transport region HTR includes the hole transport layer HTL, the hole transport layer HTL may have a thickness of about 30 Å to about 1000 Å. When the hole transport region HTR includes the electron blocking layer EBL, the electron blocking layer EBL may have a thickness of, for example, about 10 Å to about 1000 Å. When the thicknesses of the hole transport region HTR, the hole injection layer HIL, the hole transport layer HTL, and the electron blocking layer EBL satisfy the above-described ranges, satisfactory hole transport properties may be obtained without a substantial increase in driving voltage.

The hole transport region HTR may further include, in addition to the materials described herein, a charge generation material that is configured to enhance or increase conductivity. The charge generation material may be uniformly or non-uniformly dispersed in the hole transport region HTR. The charge generation material may be, for example, a p-dopant. The p-dopant may include at least one of halogenated metal compounds, quinone derivatives, metal oxides, or cyano group-containing compounds, but is not limited thereto. For example, the p-dopant may include halogenated metal compounds such as Cul and Rbl, quinone derivatives such as tetracyanoquinodimethane (TCNQ) and 2,3,5,6-tetrafluoro-7,7,8,8-tetracyanoquinodimethane (F4-TCNQ), metal oxides such as tungsten oxides and molybdenum oxides, cyano group-containing compounds such as dipyrazino[2,3-f: 2′,3′-h] quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, but is not limited thereto.

As described elsewhere herein, the hole transport region HTR may further include at least one of a buffer layer or an electron blocking layer EBL in addition to the hole injection layer HIL and the hole transport layer HTL. The buffer layer may compensate for a resonance distance according to wavelengths of light emitted from an emission layer EML, and may thus enhance or increase light emitting efficiency. Materials which may be included in the hole transport region HTR may be utilized as materials included in the buffer layer. The electron blocking layer EBL is a layer that serves to prevent or reduce electrons from being injected from the electron transport region ETR to the hole transport region HTR.

The first electrode EL1 has conductivity (e.g., is a conductor). The first electrode EL1 may be formed of a metal material, a metal alloy or a conductive compound. The first electrode EL1 may be an anode or a cathode. However, the embodiment of the present disclosure is not limited thereto. In some embodiments, the first electrode EL1 may be a pixel electrode. The first electrode EL1 may be a transmissive electrode, a transflective electrode, or a reflective electrode. The first electrode EL1 may be a transmissive electrode, a transflective electrode, or a reflective electrode. The first electrode may include at least one selected from Ag, Mg, Cu, Al, Pt, Pd, Au, Ni, Nd, Ir, Cr, Li, Ca, LiF, Mo, Ti, W, In, Sn, and Zn, two or more compounds selected therefrom, two or more mixtures selected therefrom, and/or an oxide thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stack structure of LiF and Ca), LiF/Al (a stack structure of LiF and Al), Mo, Ti, W, a compound thereof, or a mixture thereof (e.g., a mixture of Ag and Mg). In some embodiments, the first electrode EL1 may have a multilayer structure including a reflective film or a transflective film formed of the above-described materials, and a transparent conductive film formed of indium tin oxide (ITO), indium zinc oxide (IZO), zinc oxide (ZnO), indium tin zinc oxide (ITZO), and/or the like. For example, the first electrode EL1 may have a three-layer structure of ITO/Ag/ITO, but is not limited thereto. In some embodiments, the embodiment of the present disclosure is not limited thereto, and the first electrode EL1 may include the above-described metal materials, a combination of two or more metal materials selected from the above-described metal materials, and/or oxide(s) of the above-described metal materials. The first electrode EL1 may have a thickness of about 700 Å to about 10000 Å. For example, the first electrode EL1 may have a thickness of 1000 Å to about 3000 Å.

The emission layer EML is located or provided on the hole transport region HTR. The emission layer EML may have, for example, a thickness of about 100 Å to about 1000 Å or about 100 Å to about 300 Å. The emission layer EML may have a single layer formed of a single material, a single layer formed of a plurality of different materials, or a multilayer structure having a plurality of layers formed of a plurality of different materials.

In the light emitting element ED according to an embodiment, the emission layer EML may include an anthracene derivative, a pyrene derivative, a fluoranthene derivative, a chrysene derivative, a dihydrobenzanthracene derivative, and/or a triphenylene derivative. In an embodiment, the emission layer EML may include an anthracene derivative and/or a pyrene derivative.

In the light emitting element ED of the embodiment shown in FIGS. 3 to 6, the emission layer EML may include a host and a dopant, and the emission layer EML may include a compound represented by Formula E-1. In an embodiment, the compound represented by Formula E-1 may be utilized as a fluorescent host material.

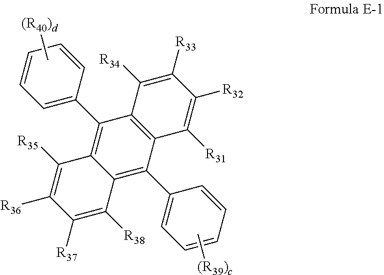

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring. In some embodiments, R₃₁ to R₄₀ may be bonded to an adjacent group to form a saturated hydrocarbon ring, an unsaturated hydrocarbon ring, a saturated heterocycle, or an unsaturated heterocycle.

In Formula E-1, c and d may each independently be an integer of 0 to 5.

Formula E-1 may be represented by any one selected from among compounds E1 to E19.

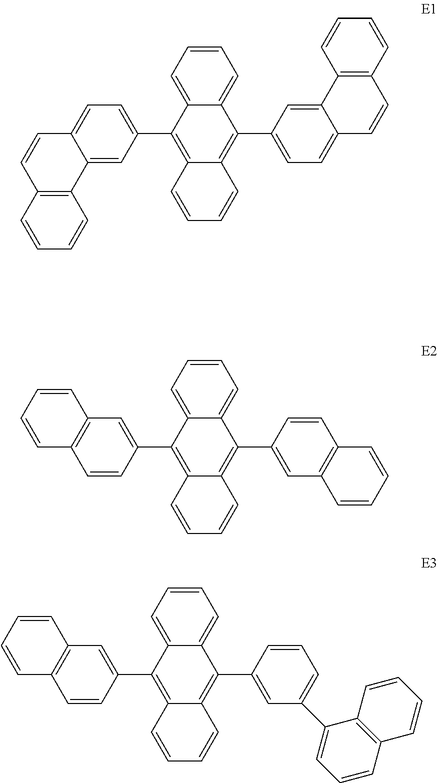

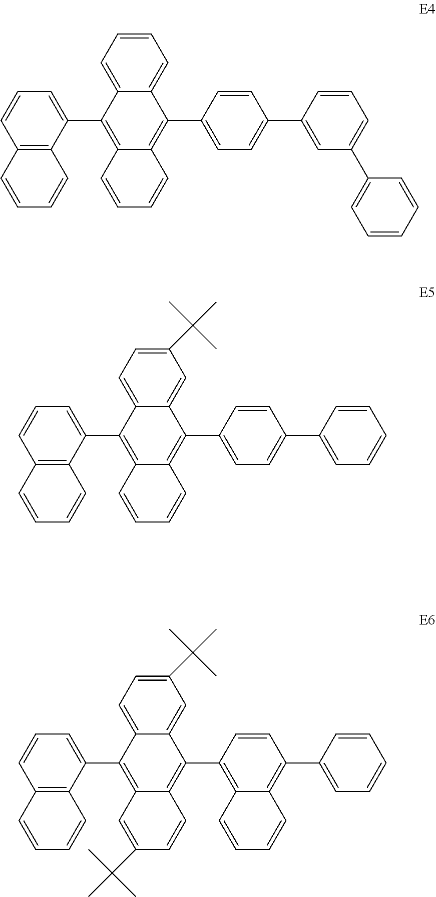

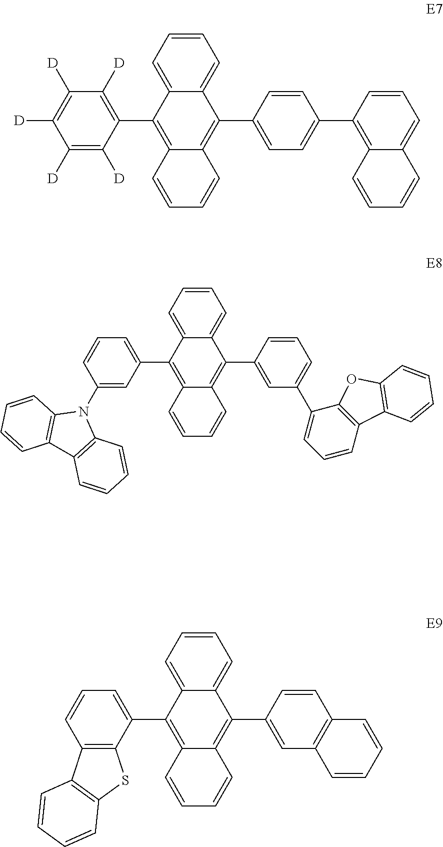 [SMILES: [2H]c1c([2H])c([2H])c(-c2c3ccccc3c(-c3ccc(-c4cccc5ccccc45)cc3)c3ccccc23)c([2H])c1[2H] ; c1cc(-c2c3ccccc3c(-c3cccc(-n4c5ccccc5c5ccccc54)c3)c3ccccc23)cc(-c2cccc3c2oc2ccccc23)c1 ; c1ccc2cc(-c3c4ccccc4c(-c4cccc5c4sc4ccccc45)c4ccccc34)ccc2c1]

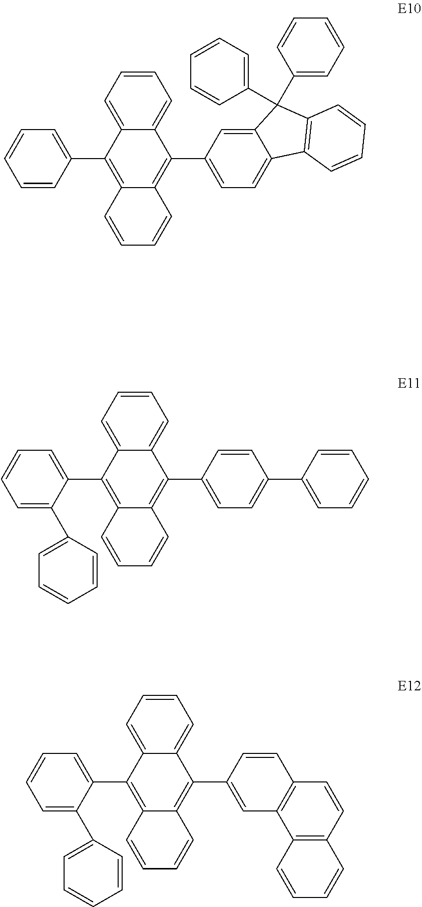

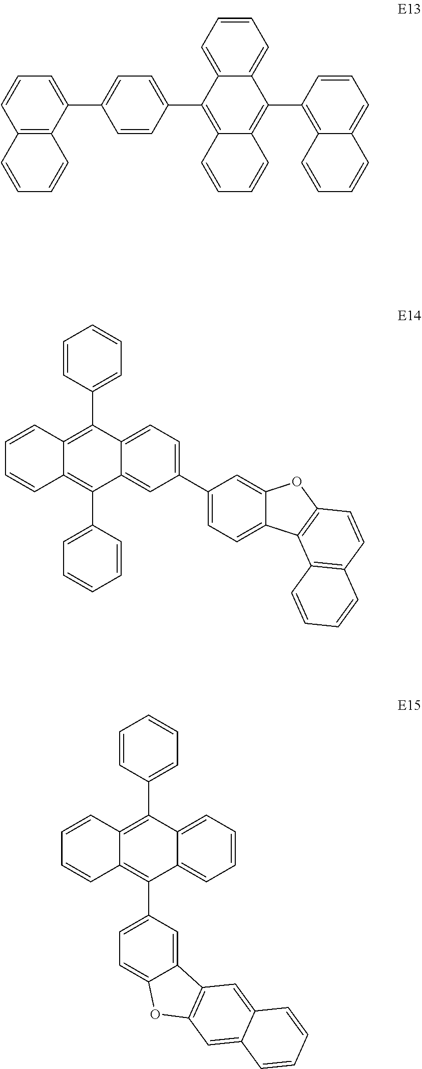

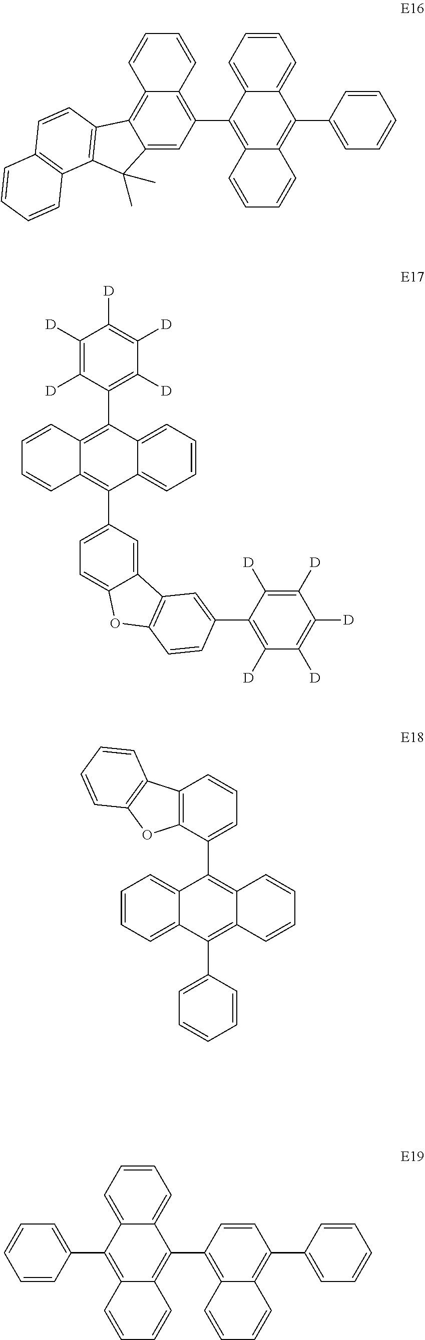

In an embodiment, the emission layer EML may include a first compound represented by Formula E-1, and at least one of a second compound represented by Formula HT-1, a third compound represented by Formula ET-1, or a fourth compound represented by Formula M-b.

In an embodiment, the second compound may be utilized as a hole transporting host material of the emission layer EML.

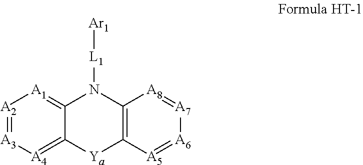

In Formula HT-1, A₁ to A₈ may be each independently N or CR₅₁. For example, all of A₁ to A₈ may be CR₅₁. Alternatively, any one among A₁ to A₈ may be N, and the rest may be CR₅₁.

In Formula HT-1, L₁ may be a direct linkage, a substituted or unsubstituted arylene group having 6 to 30 ring-forming carbon atoms, or a substituted or unsubstituted heteroarylene group having 2 to 30 ring-forming carbon atoms. For example, L₁ may be a direct linkage, a substituted or unsubstituted phenylene group, a substituted or unsubstituted divalent biphenyl group, or a substituted or unsubstituted divalent carbazole group, etc., but the embodiment of the inventive concept is not limited thereto.

In Formula HT-1, Yₐ may be a direct linkage, CR₅₂R₅₃, or SiR₅₄R₅₅. That is, it may mean that the two benzene rings linked to the nitrogen atom in Formula HT-1 are linked via a direct linkage,

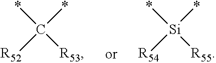

In Formula HT-1, when Yₐ is a direct linkage, the second compound represented by Formula HT-1 may include a carbazole moiety.

In Formula HT-1, Ar₁ may be a substituted or unsubstituted aryl group having 6 to 30 ring-forming carbon atoms, or a substituted or unsubstituted heteroaryl group having 2 to 30 ring-forming carbon atoms. For example, Ar₁ may be a substituted or unsubstituted carbazole group, a substituted or unsubstituted dibenzofuran group, a substituted or unsubstituted dibenzothiophene group, a substituted or unsubstituted biphenyl group, etc., but the embodiment of the inventive concept is not limited thereto.

In Formula HT-1, R₅₁ to R₅₅ may be each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms. Alternatively, each of R₅₁ to R₅₅ may be bonded to an adjacent group to form a ring. For example, R₅₁ to R₅₅ may be each independently a hydrogen atom or a deuterium atom. R₅₁ to R₅₅ may be each independently an unsubstituted methyl group or an unsubstituted phenyl group.

The second compound may be represented by any one selected from among compounds of Compound Group 2. In Compound Group 2, D may be a deuterium atom.

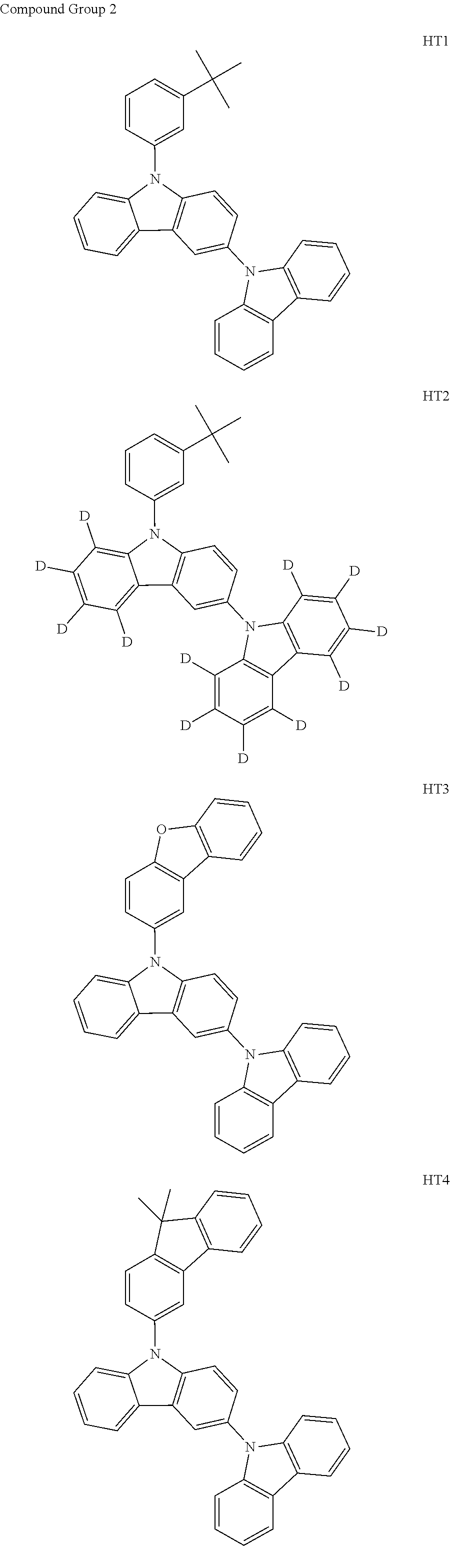

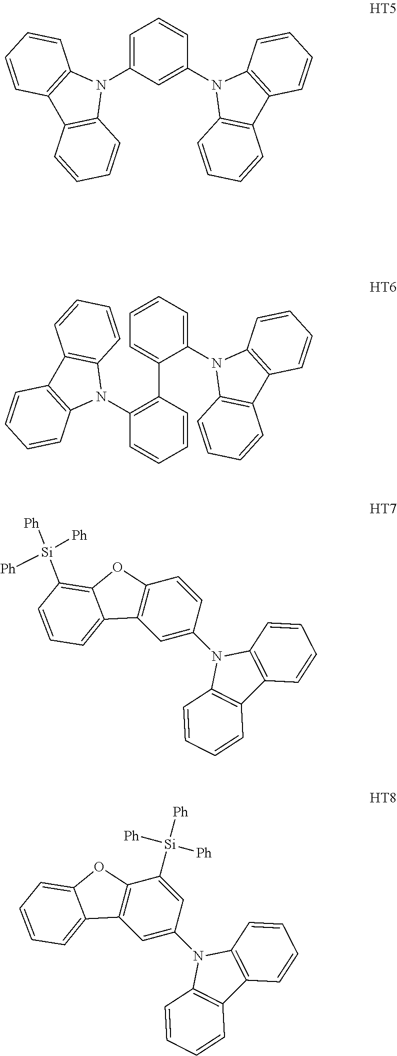 [SMILES: c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1 ; c1ccc([Si](c2ccccc2)(c2ccccc2)c2cc(-n3c4ccccc4c4ccccc43)cc3c2oc2ccccc23)cc1]

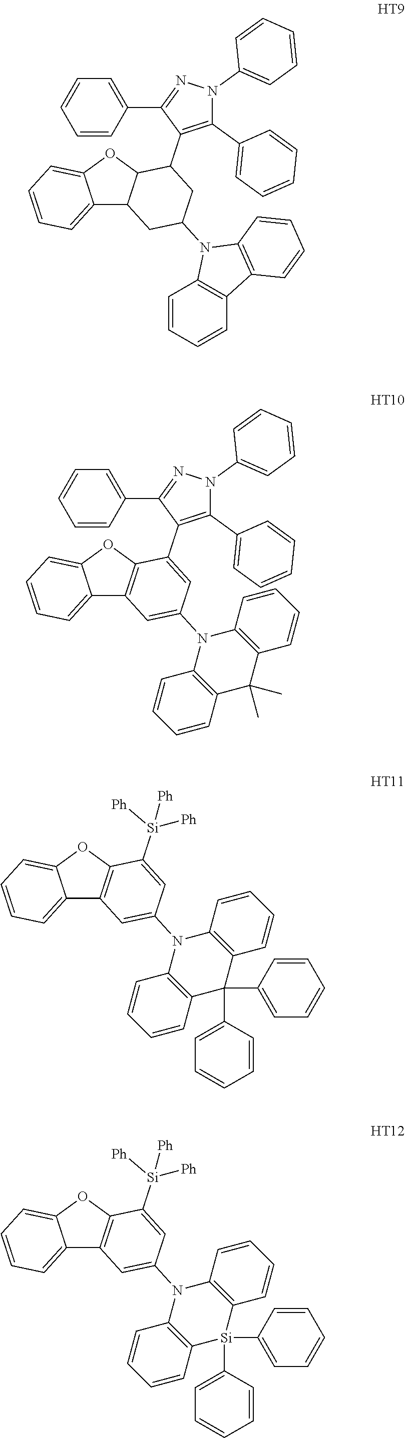 [SMILES: c1ccc(-c2nn(-c3ccccc3)c(-c3ccccc3)c2C2CC(n3c4ccccc4c4ccccc43)CC3c4ccccc4OC32)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1 ; c1ccc([Si]2(c3ccccc3)c3ccccc3N(c3cc([Si](c4ccccc4)(c4ccccc4)c4ccccc4)c4oc5ccccc5c4c3)c3ccccc32)cc1]

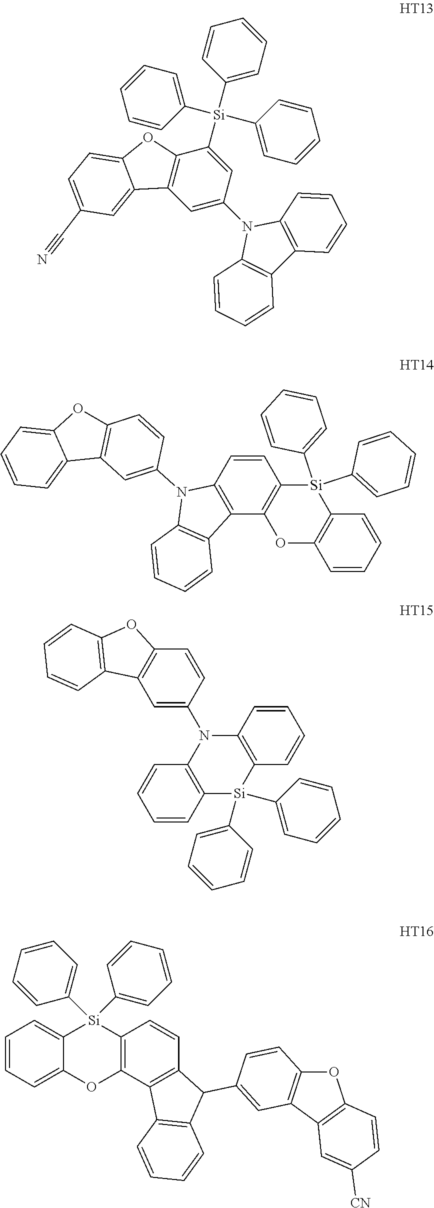 [SMILES: N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1 ; c1ccc([Si]2(c3ccccc3)c3ccccc3N(c3ccc4oc5ccccc5c4c3)c3ccccc32)cc1 ; N#Cc1ccc2oc3ccc(C4c5ccccc5-c5c4ccc4c5Oc5ccccc5[Si]4(c4ccccc4)c4ccccc4)cc3c2c1]

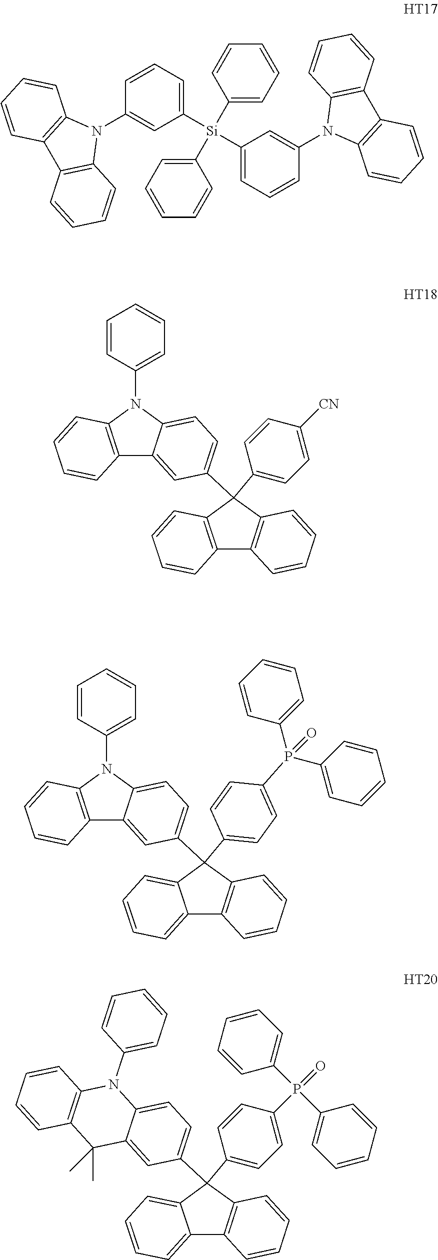 [SMILES: c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21]

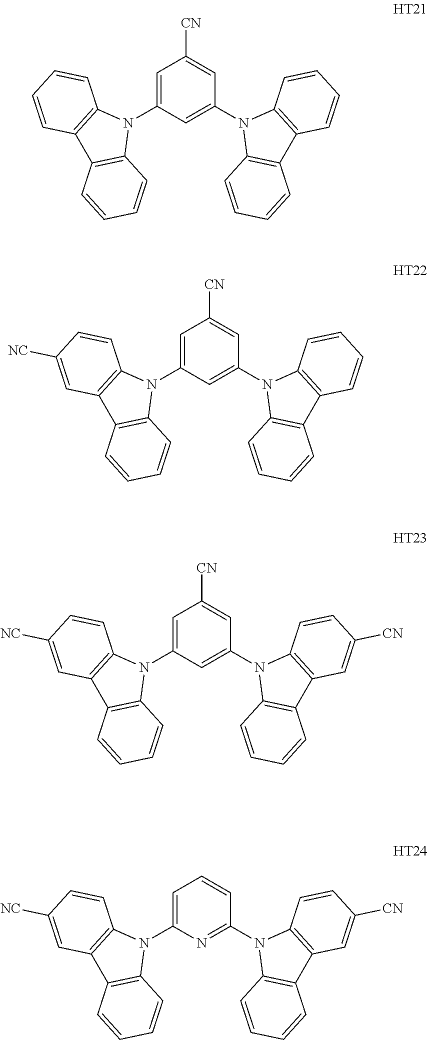 [SMILES: N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1]

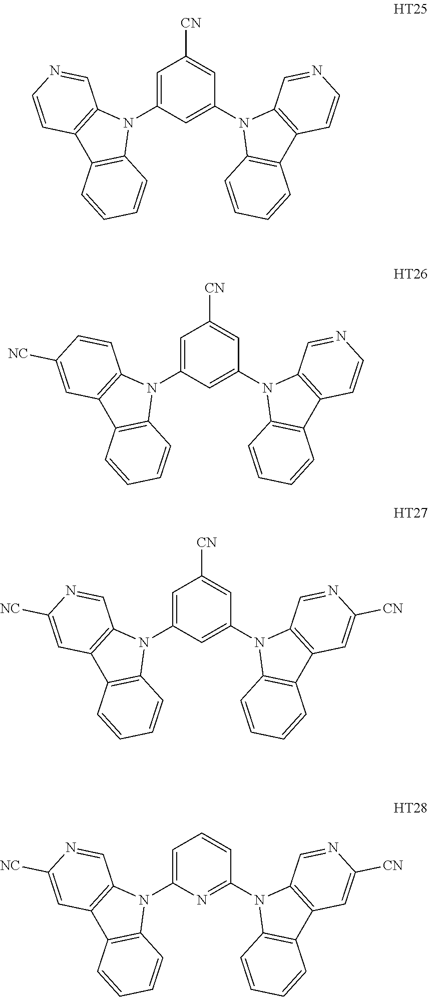 [SMILES: N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1]

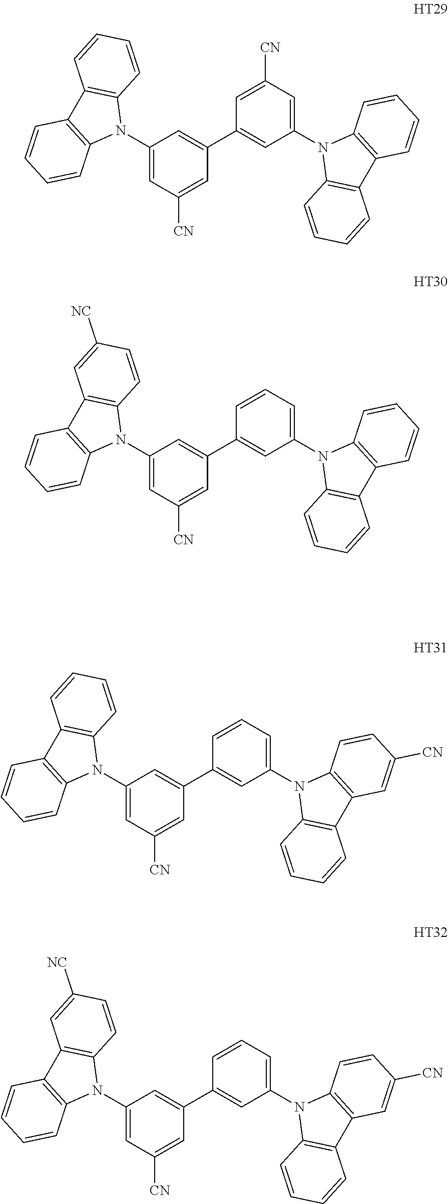 [SMILES: N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1 ; N#Cc1cc(-c2cccc(-n3c4ccccc4c4cc(C#N)ccc43)c2)cc(-n2c3ccccc3c3cc(C#N)ccc32)c1]

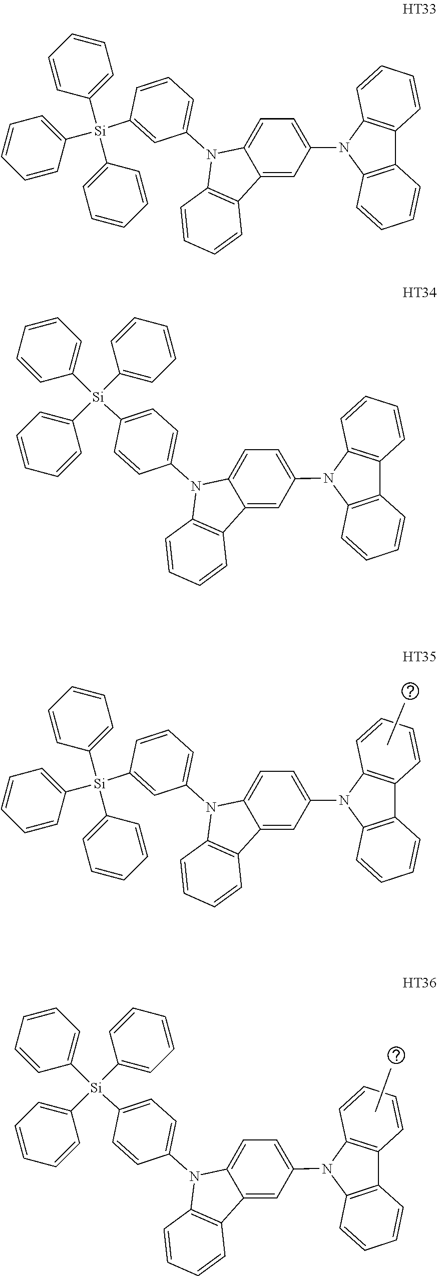

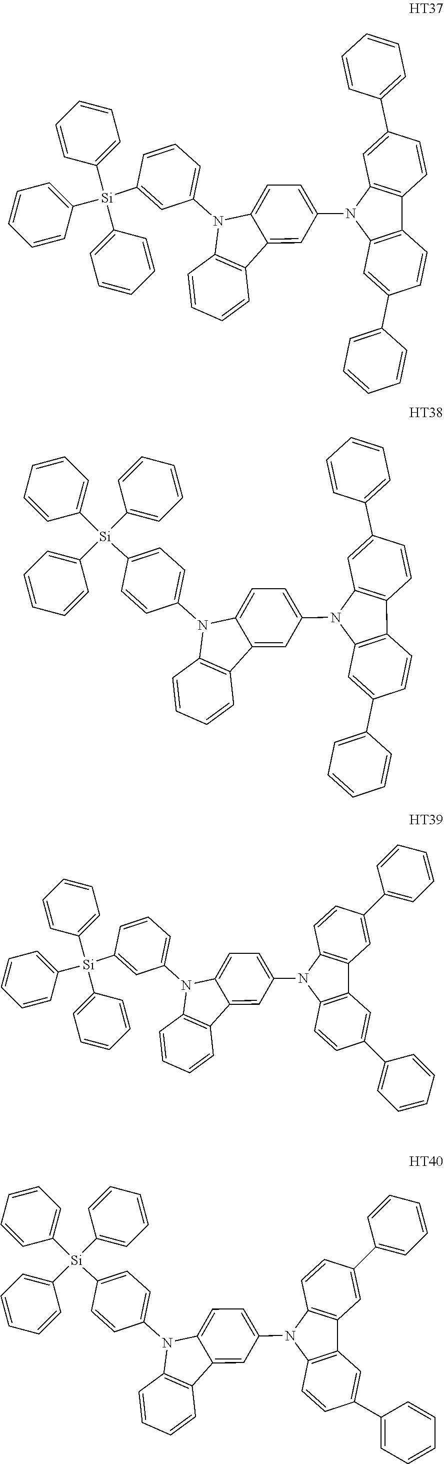 [SMILES: c1ccc(-c2ccc3c4ccc(-c5ccccc5)cc4n(-c4ccc5c(c4)c4ccccc4n5-c4cccc([Si](c5ccccc5)(c5ccccc5)c5ccccc5)c4)c3c2)cc1 ; c1ccc(-c2ccc3c4ccc(-c5ccccc5)cc4n(-c4ccc5c(c4)c4ccccc4n5-c4ccc([Si](c5ccccc5)(c5ccccc5)c5ccccc5)cc4)c3c2)cc1 ; c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1]

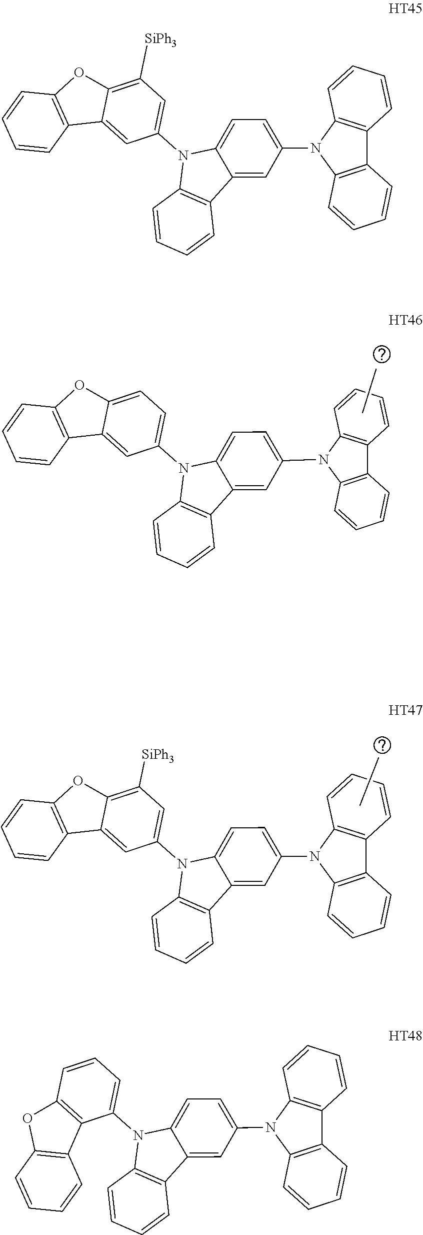

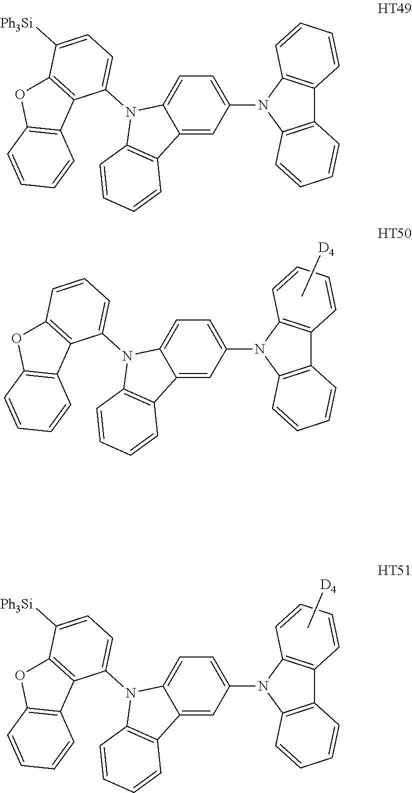

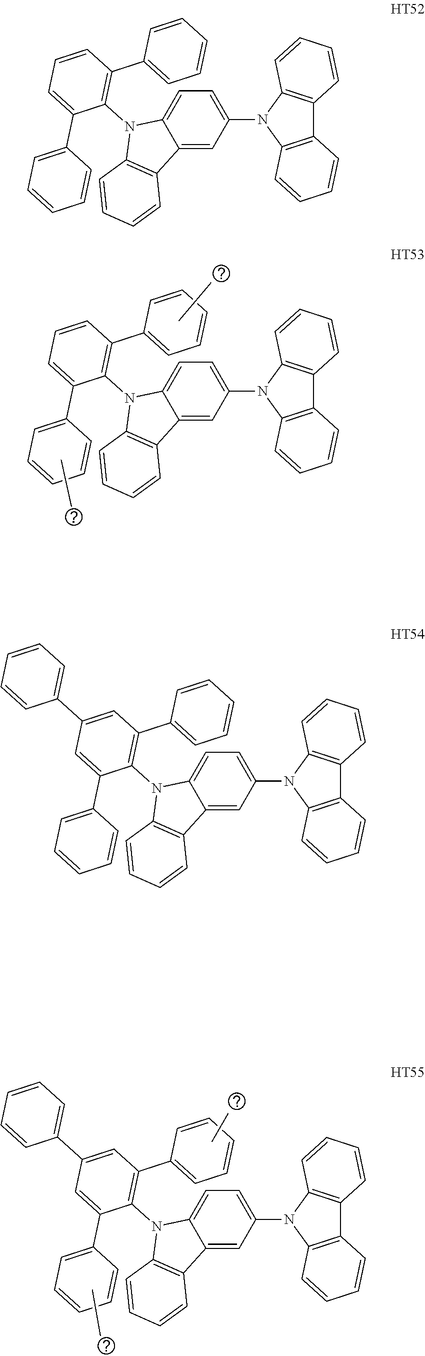

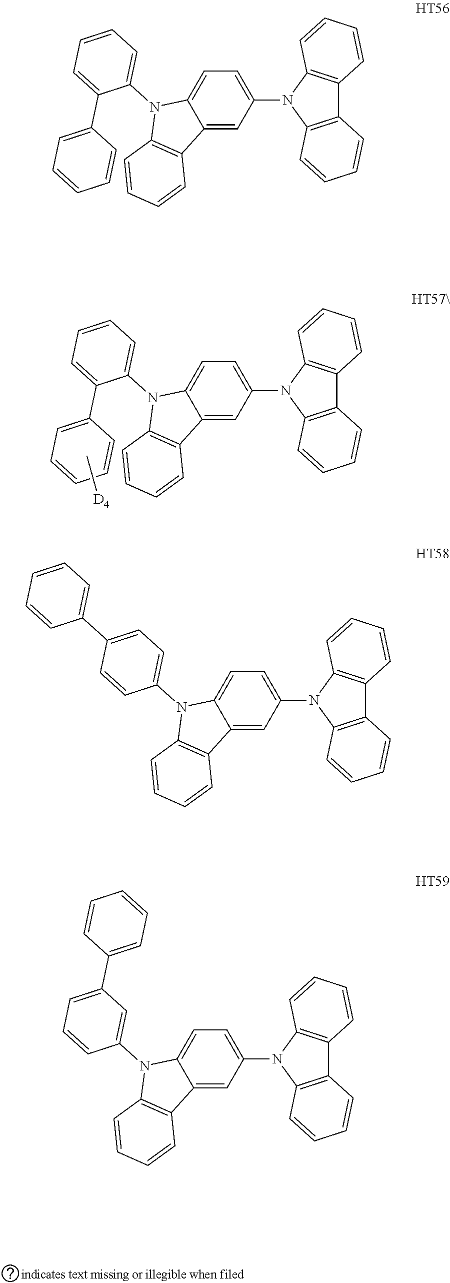

In an embodiment, the emission layer EML may include a third compound represented by Formula ET-1. For example, the third compound may be utilized as an electron transporting host material of the emission layer EML.

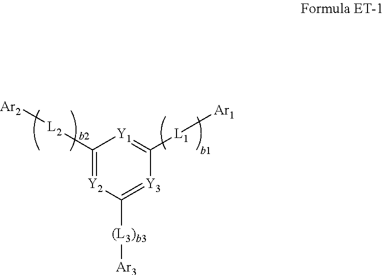

In Formula ET-1, at least one of Y₁ to Y₃ may be N, and the others may be CRₐ, and Rₐ may be a hydrogen atom, a deuterium atom, a substituted or unsubstituted alkyl group having 1 to 20 carbon atoms, a substituted or unsubstituted aryl group having 6 to 60 ring-forming carbon atoms, or a substituted or unsubstituted heteroaryl group having 2 to 60 ring-forming carbon atoms.

In Formula ET-1, b1 to b3 may each independently be an integer of 0 to 10. L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms.

In Formula ET-1, Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, Ar₁ to Ar₃ may a substituted or unsubstituted phenyl group or a substituted or unsubstituted carbazole group.

The third compound may be represented by any one selected from among compounds of Compound Group 3. The light emitting element ED according to an embodiment may include any one selected from among compounds of Compound Group 3. In Compound Group 3, D may be a deuterium atom.

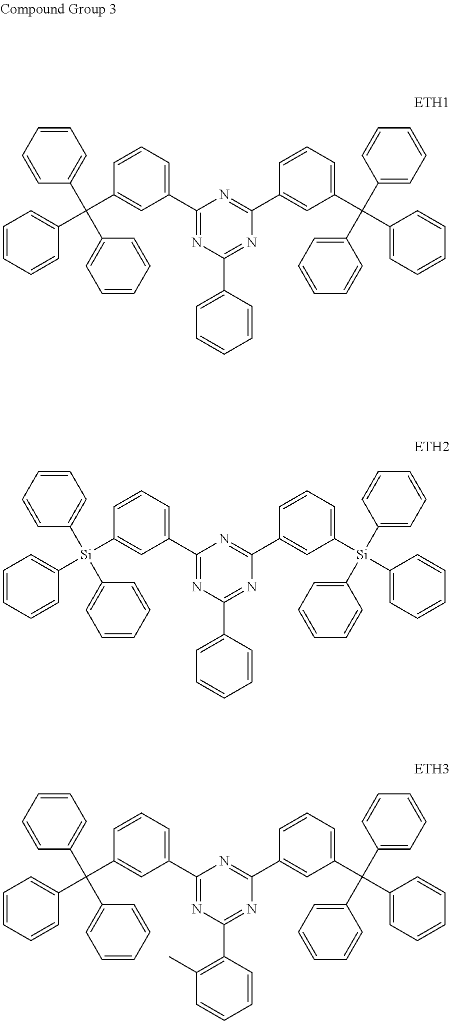 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1]

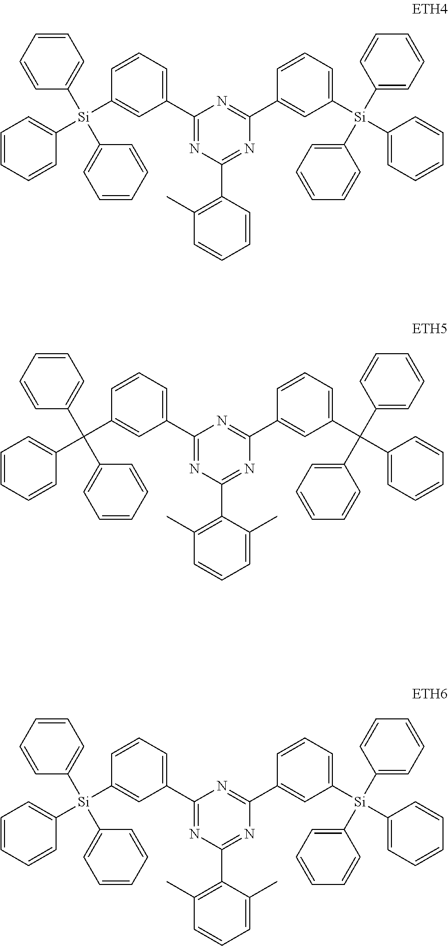 [SMILES: Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

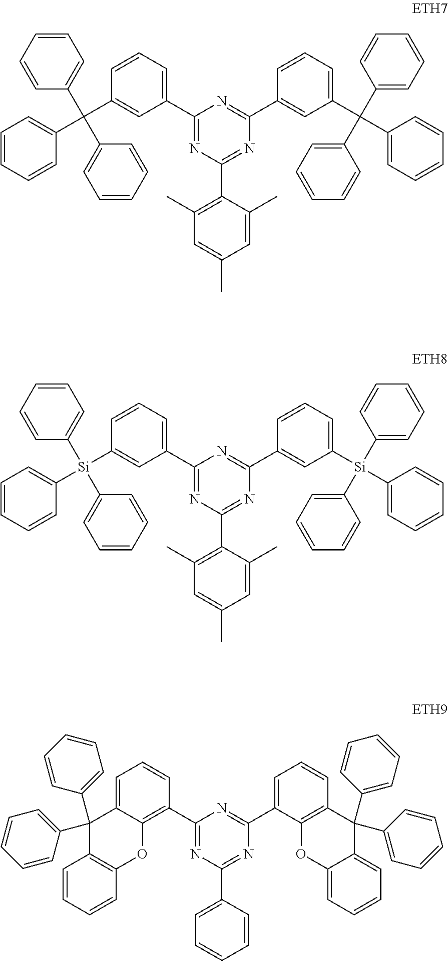 [SMILES: Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1 ; c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1]

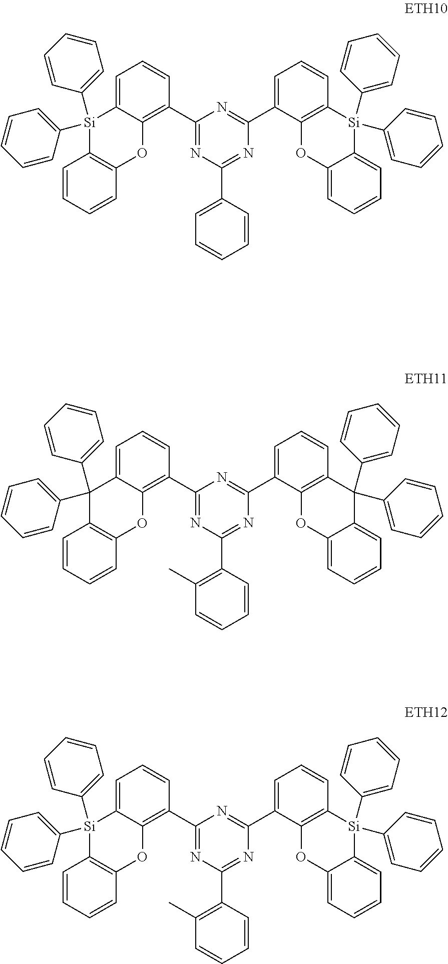 [SMILES: c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

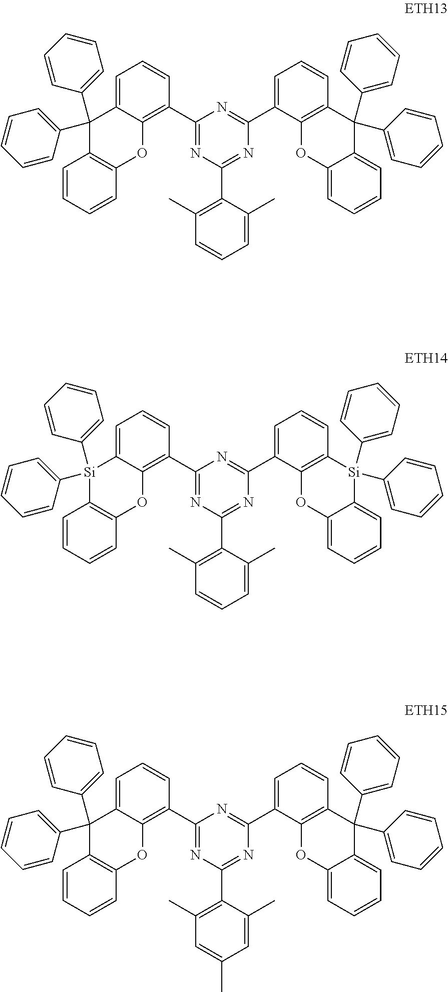 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1]

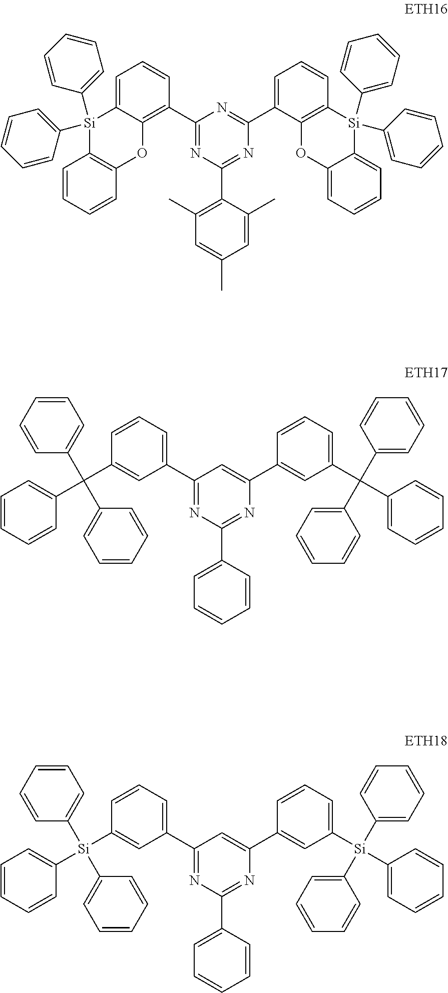 [SMILES: Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1 ; c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1]

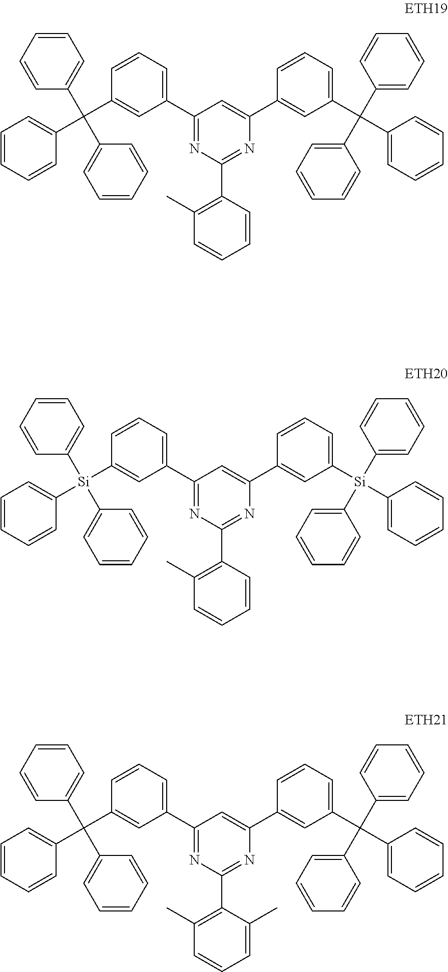 [SMILES: Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1]

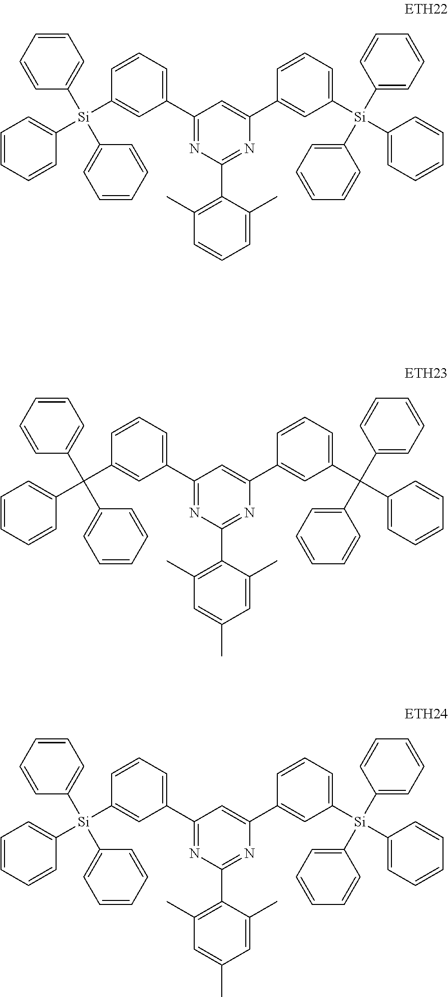 [SMILES: Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

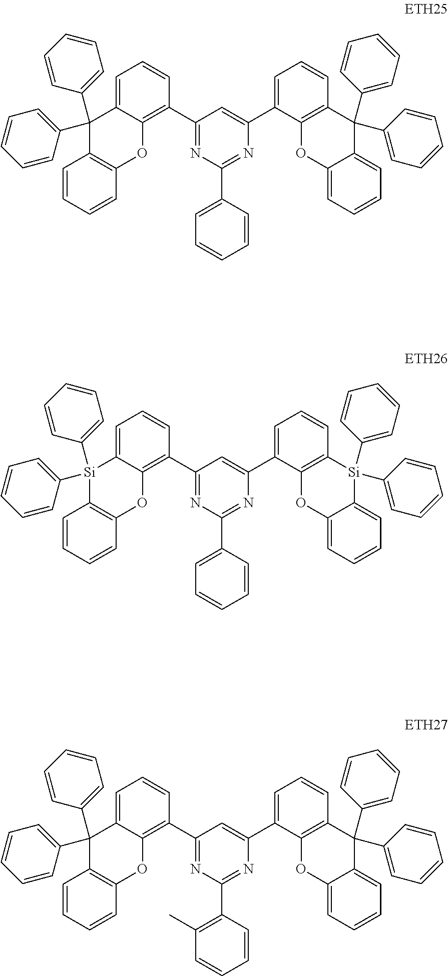 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1]

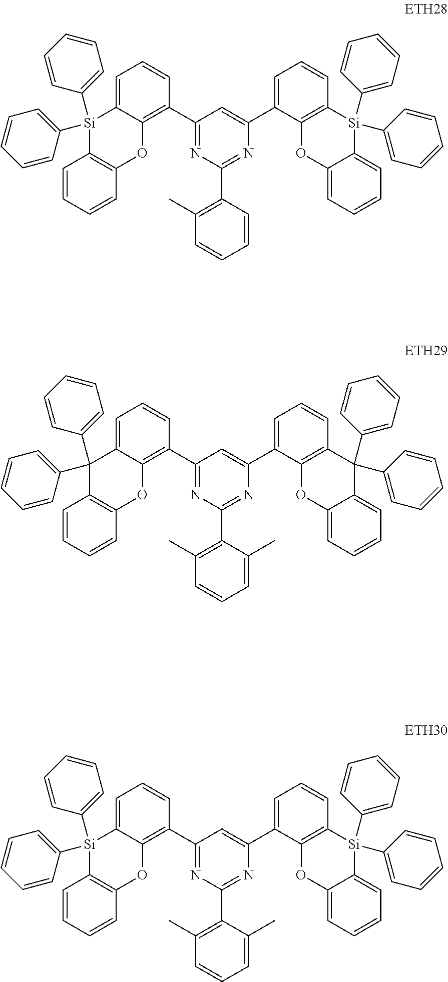 [SMILES: Cc1ccccc1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1 ; Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

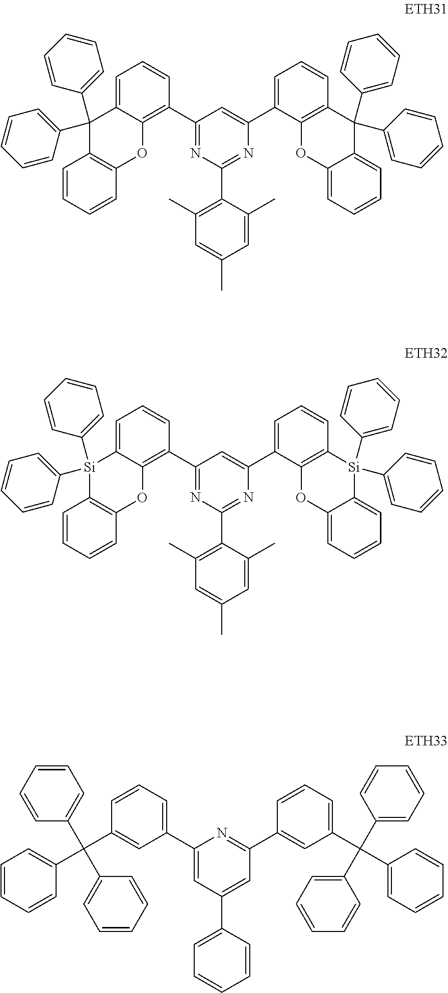 [SMILES: Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1 ; c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1]

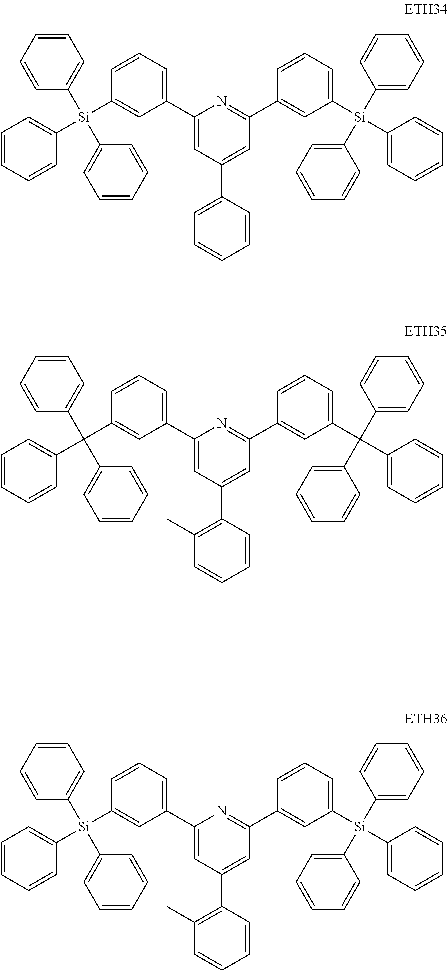 [SMILES: c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

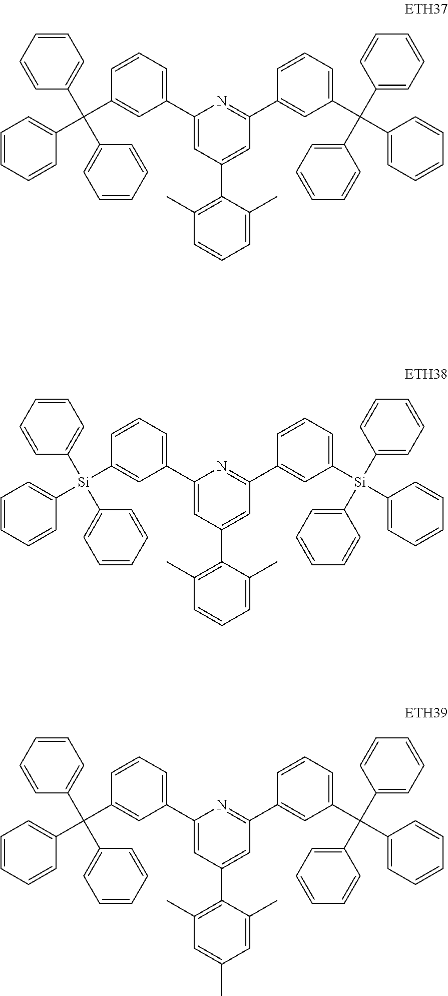 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1]

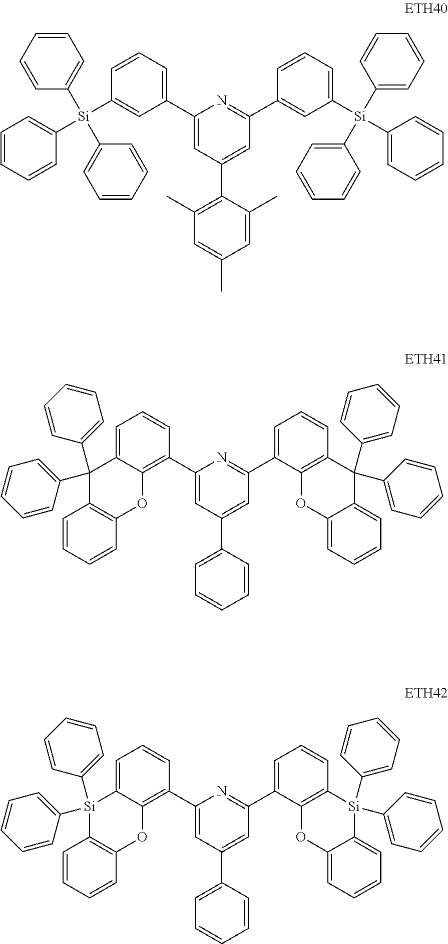 [SMILES: Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1 ; c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1]

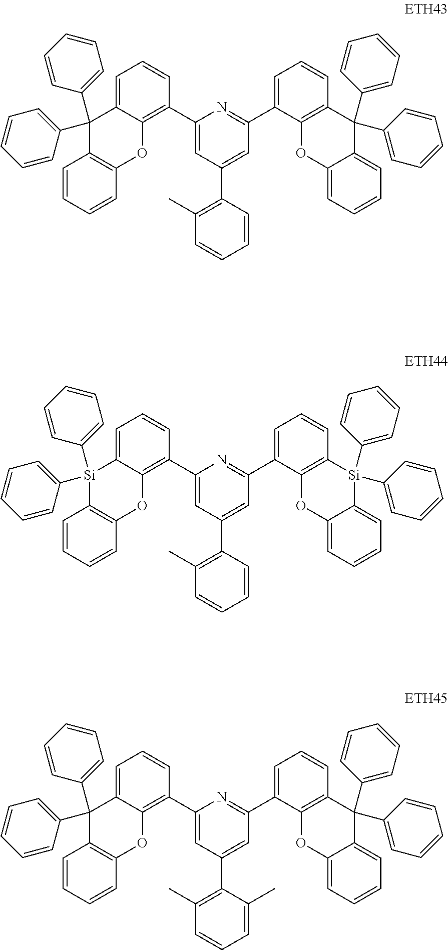 [SMILES: Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1]

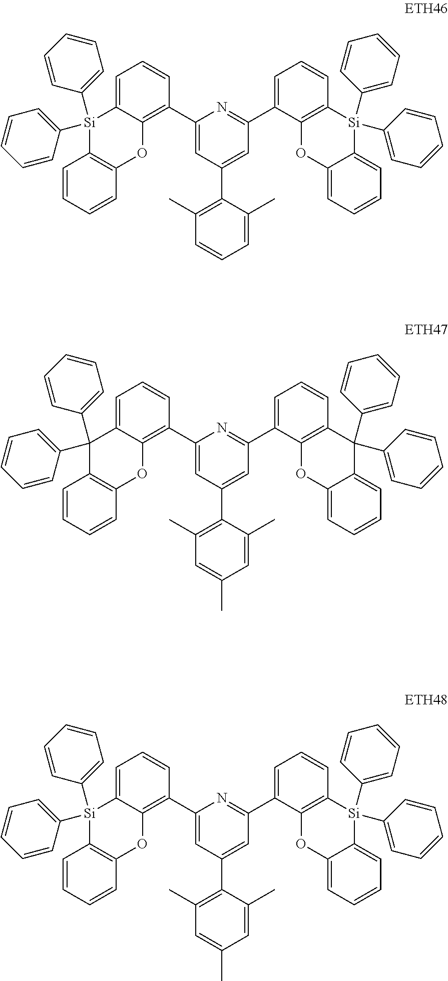 [SMILES: Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

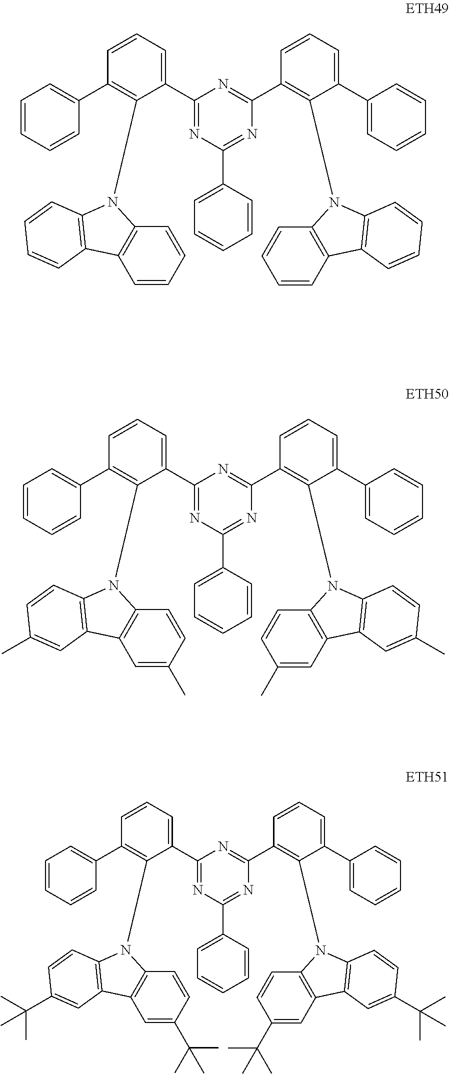

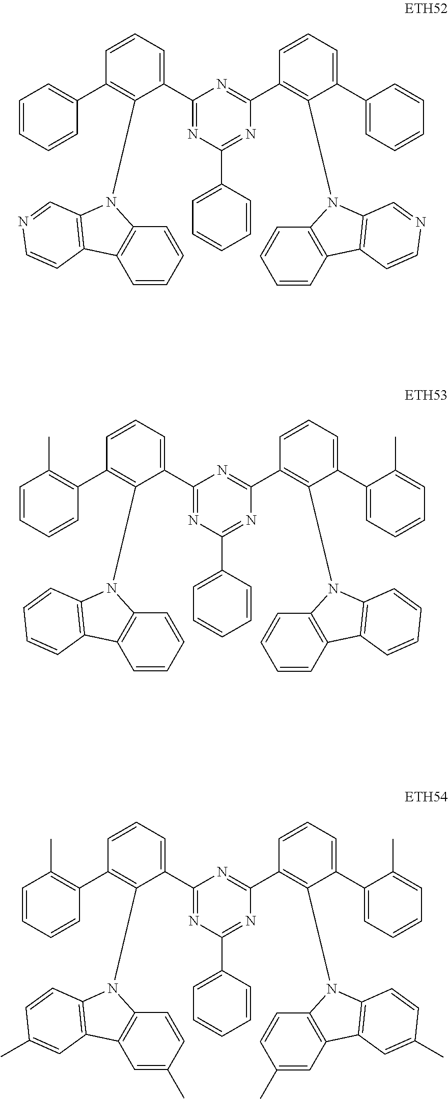

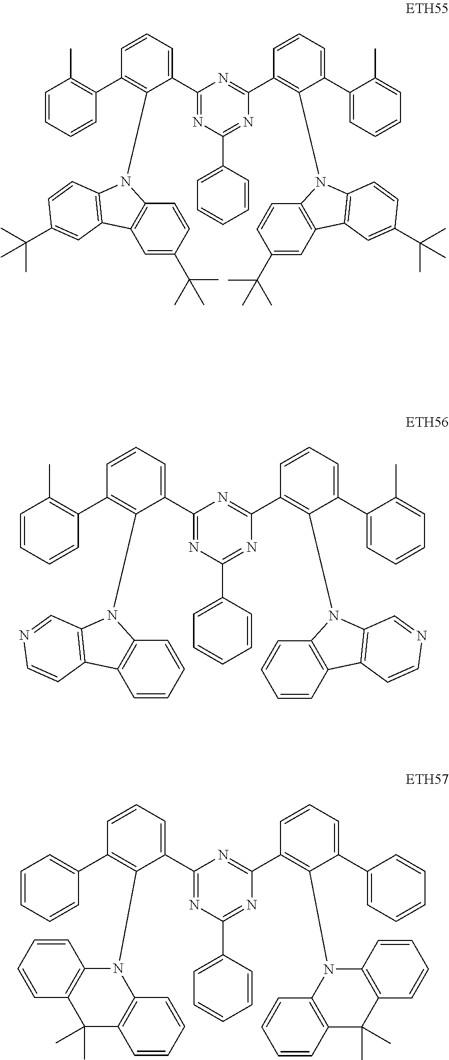 [SMILES: Cc1ccccc1-c1cccc(-c2nc(-c3ccccc3)nc(-c3cccc(-c4ccccc4C)c3-n3c4ccc(C(C)(C)C)cc4c4cc(C(C)(C)C)ccc43)n2)c1-n1c2ccc(C(C)(C)C)cc2c2cc(C(C)(C)C)ccc21 ; Cc1ccccc1-c1cccc(-c2nc(-c3ccccc3)nc(-c3cccc(-c4ccccc4C)c3-n3c4ccccc4c4ccncc43)n2)c1-n1c2ccccc2c2ccncc21 ; CC1(C)c2ccccc2N(c2c(-c3ccccc3)cccc2-c2nc(-c3ccccc3)nc(-c3cccc(-c4ccccc4)c3N3c4ccccc4C(C)(C)c4ccccc43)n2)c2ccccc21]

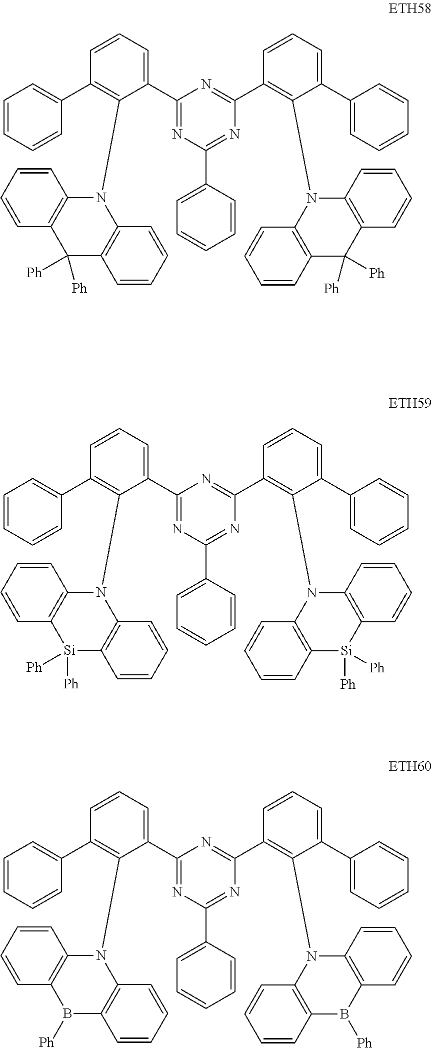 [SMILES: c1ccc(-c2nc(-c3cccc(-c4ccccc4)c3N3c4ccccc4C(c4ccccc4)(c4ccccc4)c4ccccc43)nc(-c3cccc(-c4ccccc4)c3N3c4ccccc4C(c4ccccc4)(c4ccccc4)c4ccccc43)n2)cc1 ; c1ccc(-c2nc(-c3cccc(-c4ccccc4)c3N3c4ccccc4[Si](c4ccccc4)(c4ccccc4)c4ccccc43)nc(-c3cccc(-c4ccccc4)c3N3c4ccccc4[Si](c4ccccc4)(c4ccccc4)c4ccccc43)n2)cc1 ; c1ccc(B2c3ccccc3N(c3c(-c4ccccc4)cccc3-c3nc(-c4ccccc4)nc(-c4cccc(-c5ccccc5)c4N4c5ccccc5B(c5ccccc5)c5ccccc54)n3)c3ccccc32)cc1]

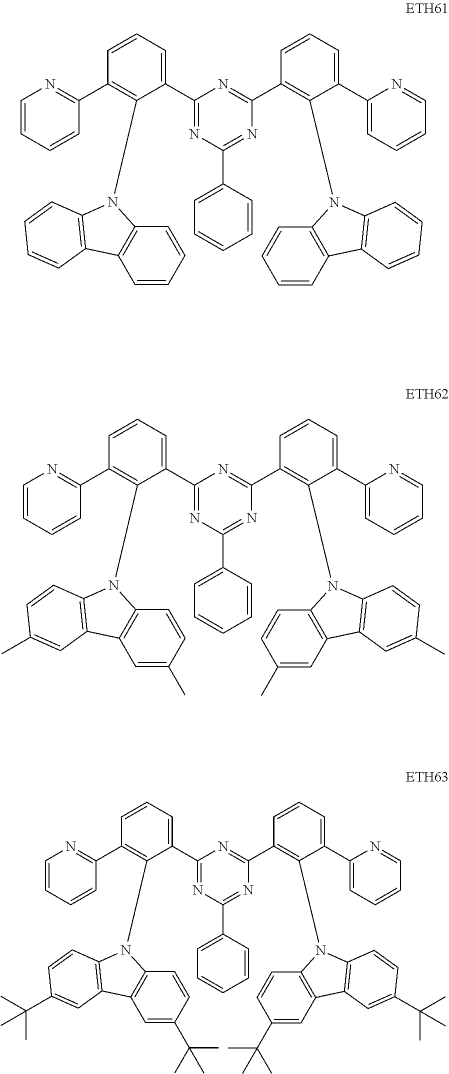

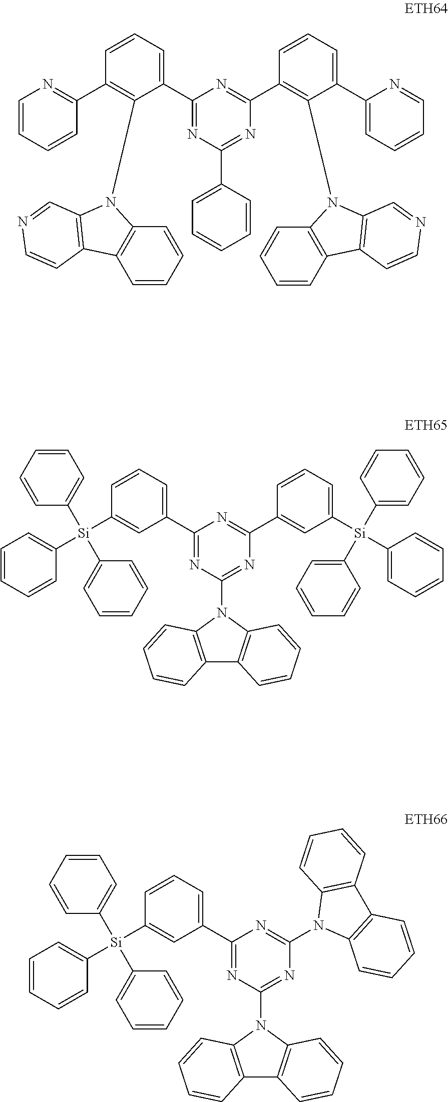 [SMILES: c1ccc(-c2nc(-c3cccc(-c4ccccn4)c3-n3c4ccccc4c4ccncc43)nc(-c3cccc(-c4ccccn4)c3-n3c4ccccc4c4ccncc43)n2)cc1 ; c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1]

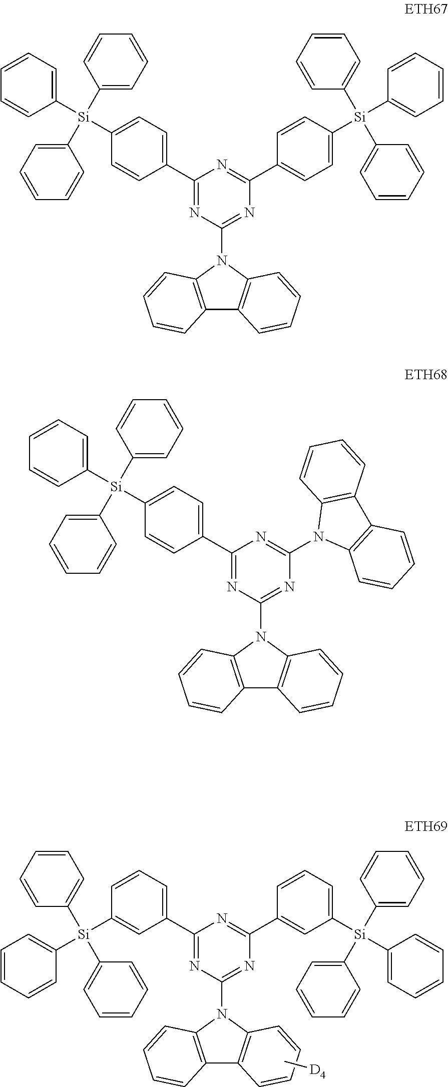

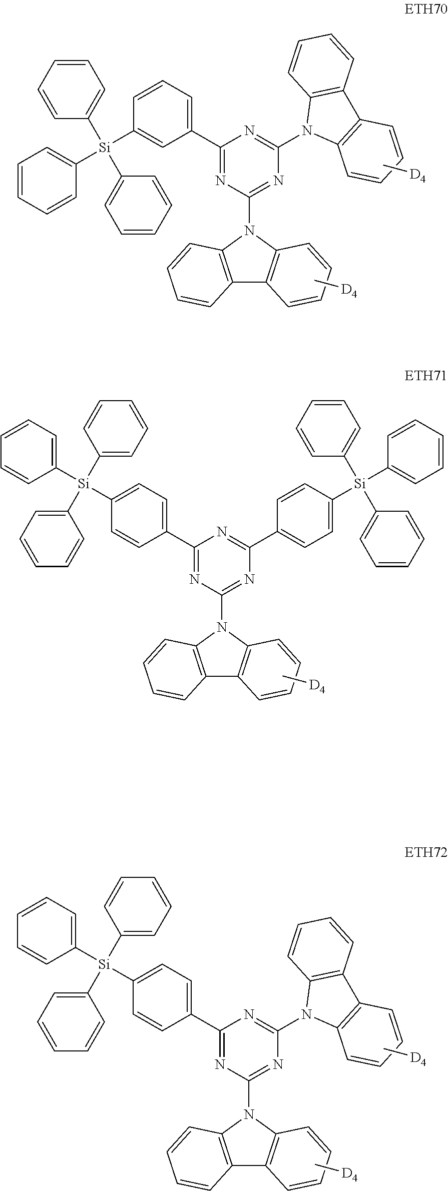

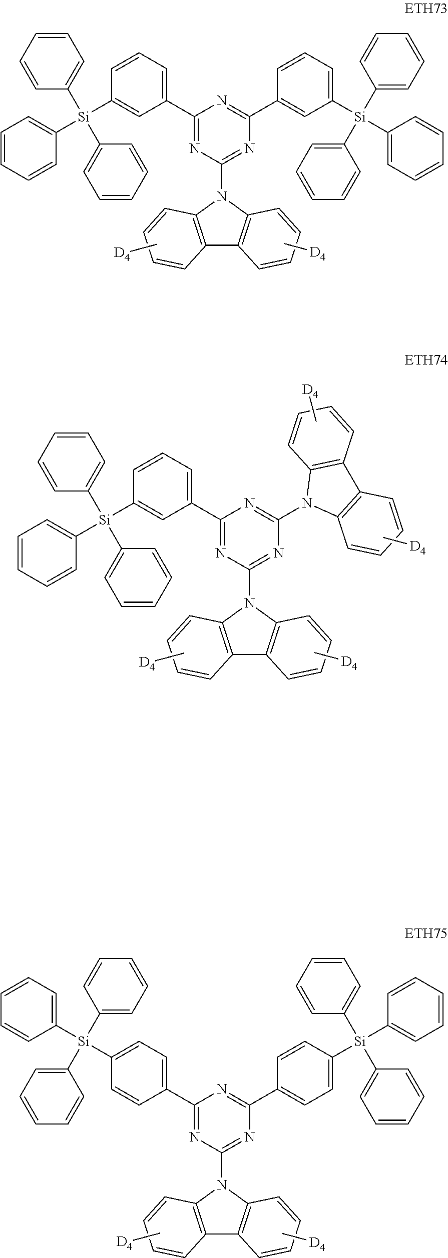

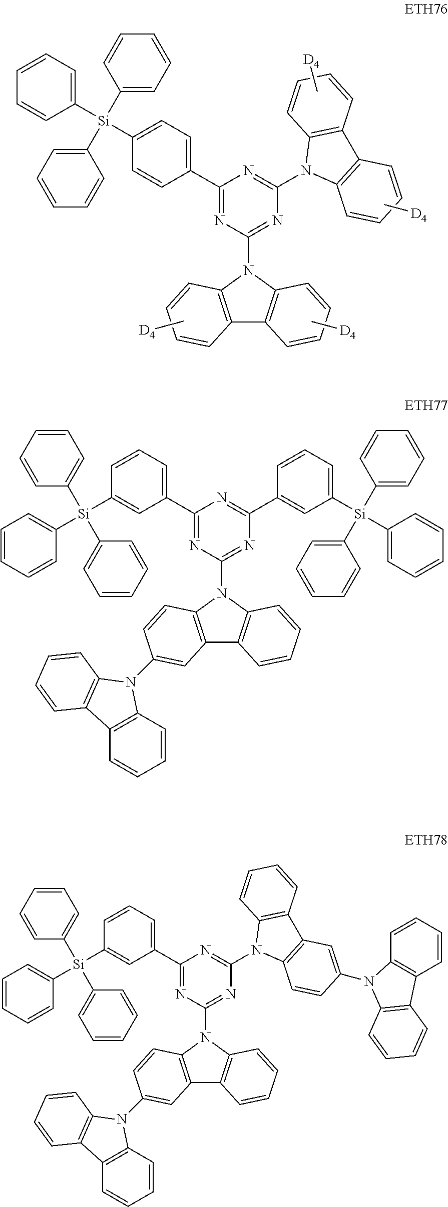

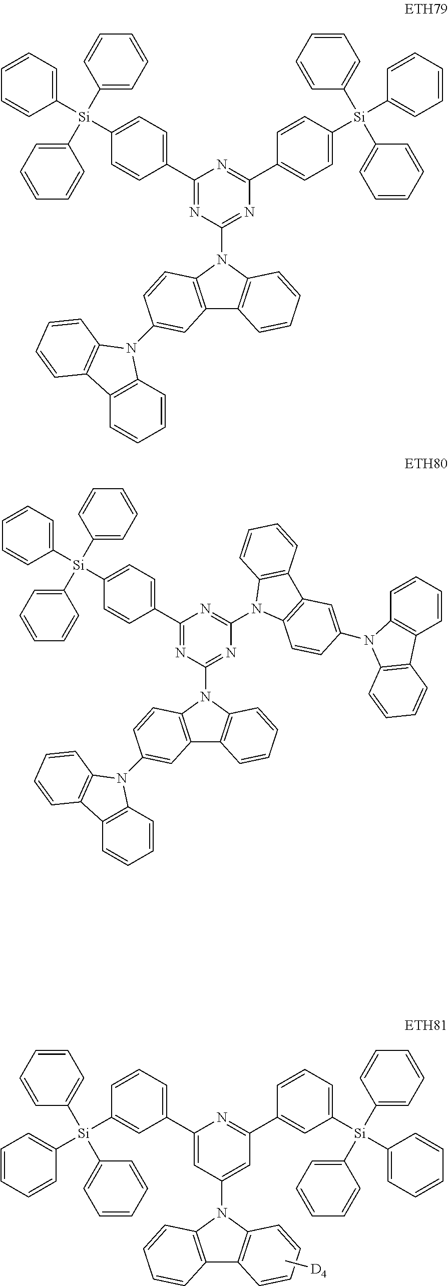

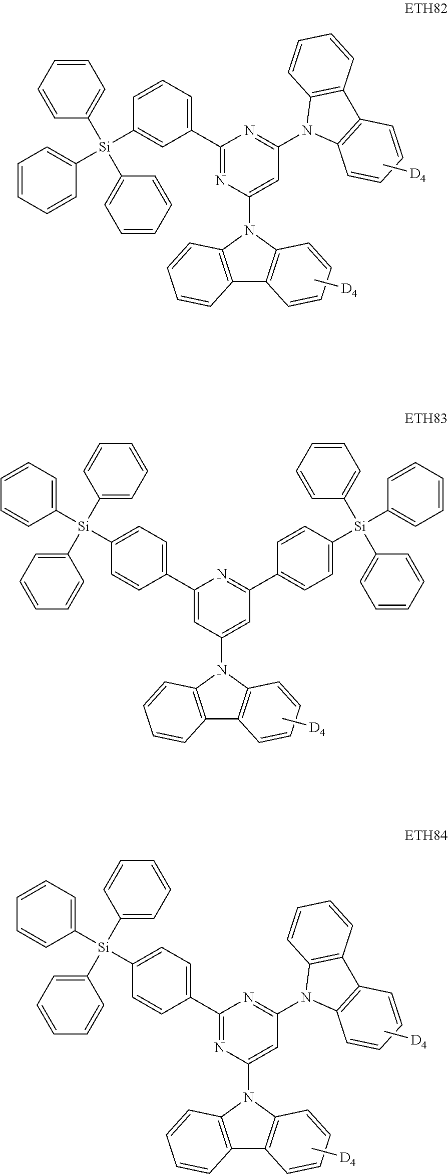

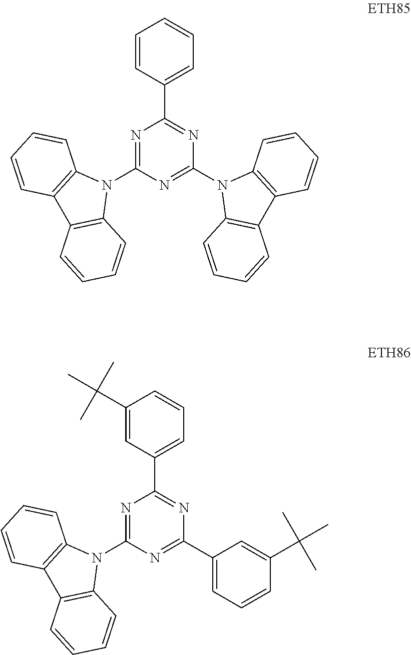 [SMILES: c1ccc(-c2nc(-n3c4ccccc4c4ccccc43)nc(-n3c4ccccc4c4ccccc43)n2)cc1 ; CC(C)(C)c1cccc(-c2nc(-c3cccc(C(C)(C)C)c3)nc(-n3c4ccccc4c4ccccc43)n2)c1]

In an embodiment, the emission layer EML may include a compound represented by Formula E-2a or Formula E-2b. The compound represented by Formula E-2a or Formula E-2b may be utilized as a phosphorescent host material.

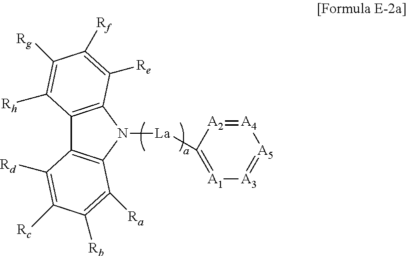

In Formula E-2a, a may be an integer of 0 to 10, and La may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or greater, a plurality of La groups may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may each independently be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring. Rₐ to Rᵢ may be bonded to an adjacent group to form a hydrocarbon ring or a heterocycle containing N, O, S, and/or the like as a ring-forming atom.

In some embodiments, in Formula E-2a, two or three selected from A₁ to A₅ may be N, and the others may be CRᵢ.

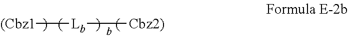

In Formula E-2b, Cbz1 and Cbz2 may each independently be an unsubstituted carbazole group or an aryl-substituted carbazole group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms, b may be an integer of 0 to 10, and when b is an integer of 2 or greater, a plurality of L_{b} groups may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one selected from among compounds from Compound Group E-2. However, the compounds listed in Compound Group E-2 are presented as an example, and the compound represented by Formula E-2a or Formula E-2b is not limited to those listed in Compound Group E-2.

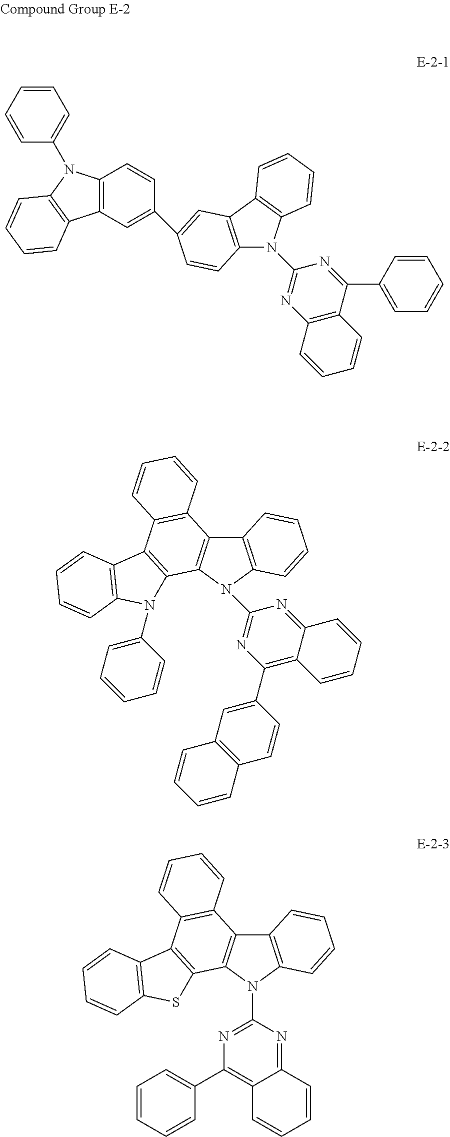 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

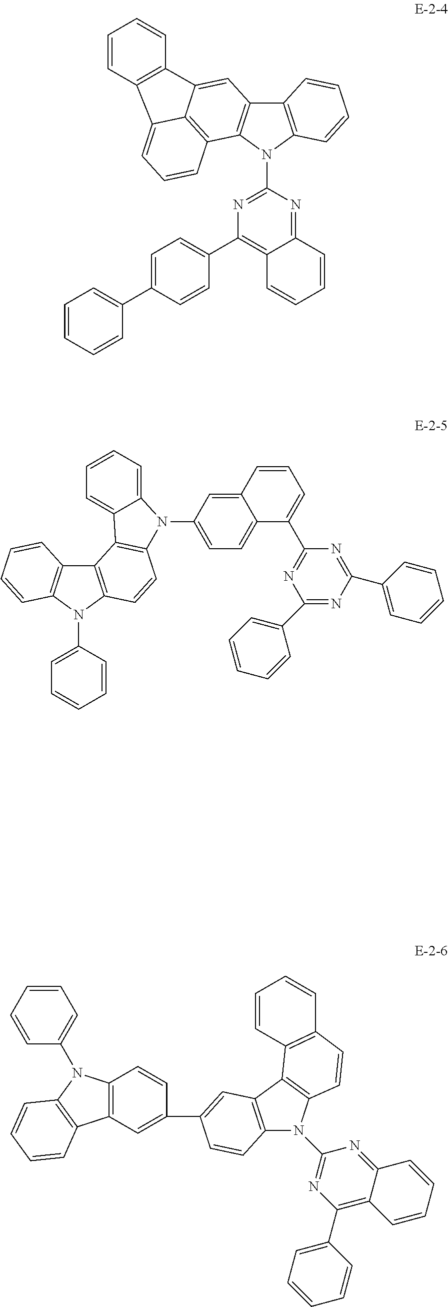 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

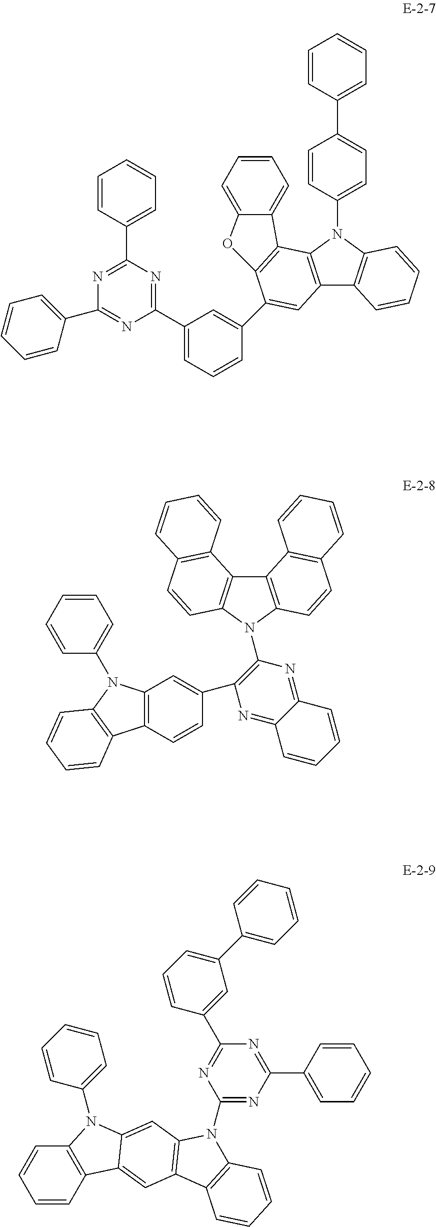 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

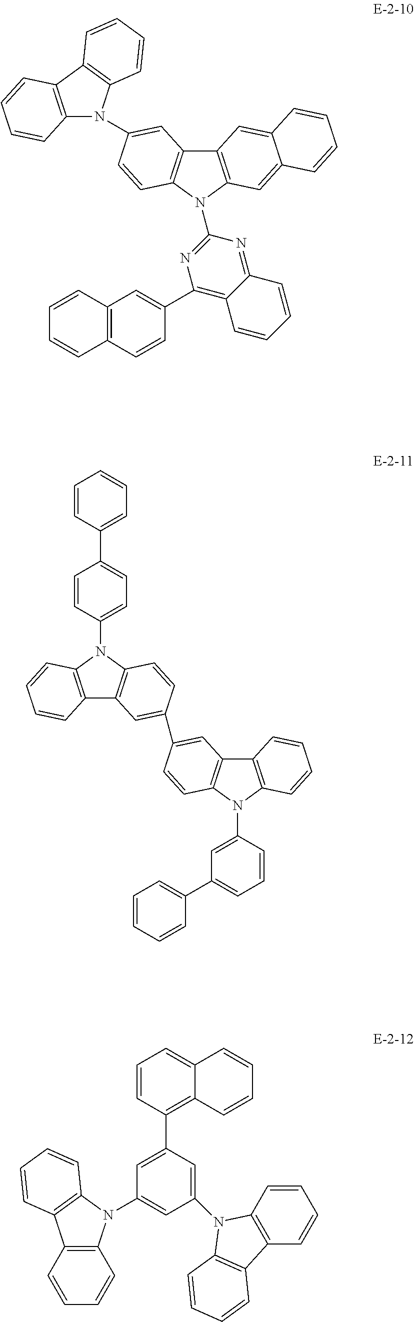 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

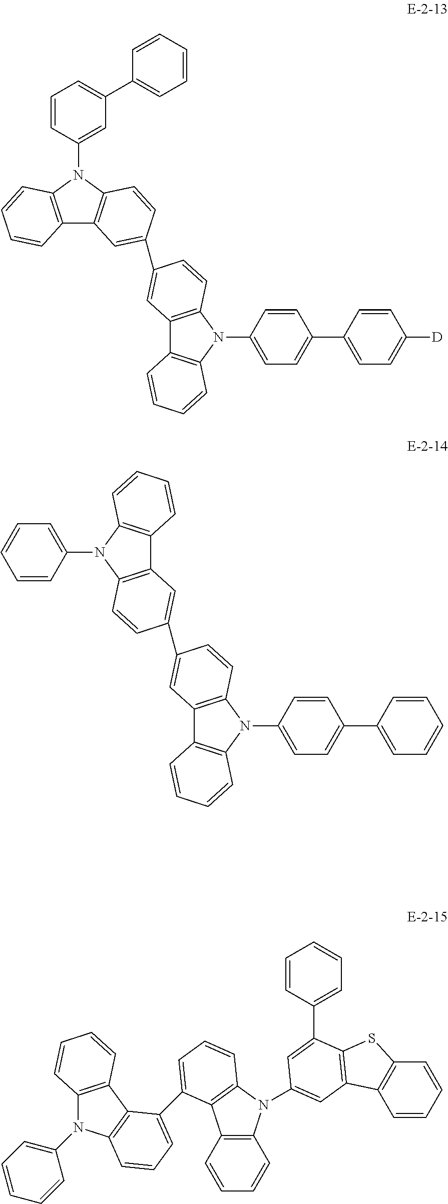 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

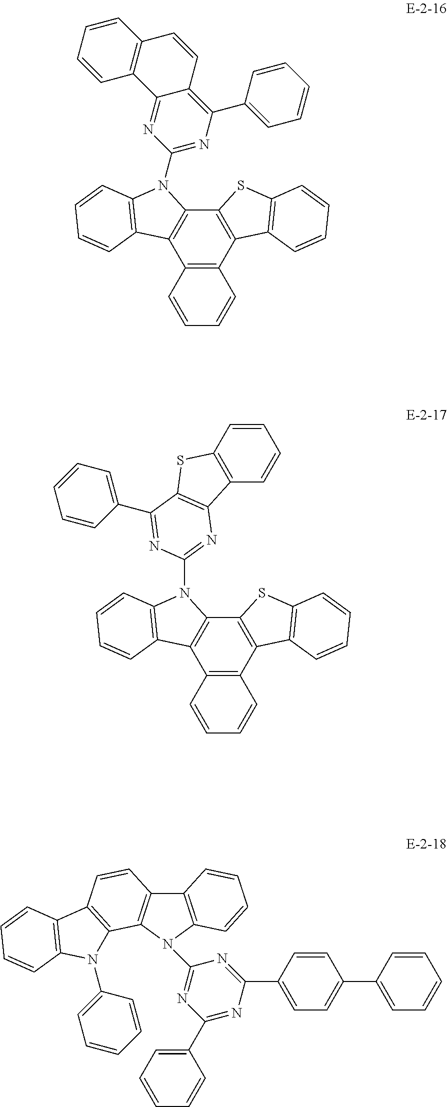 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

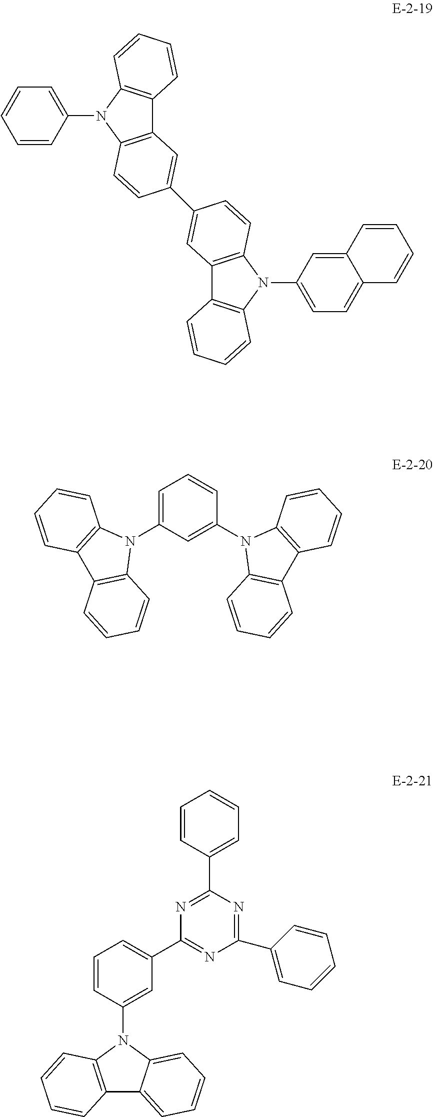 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1]

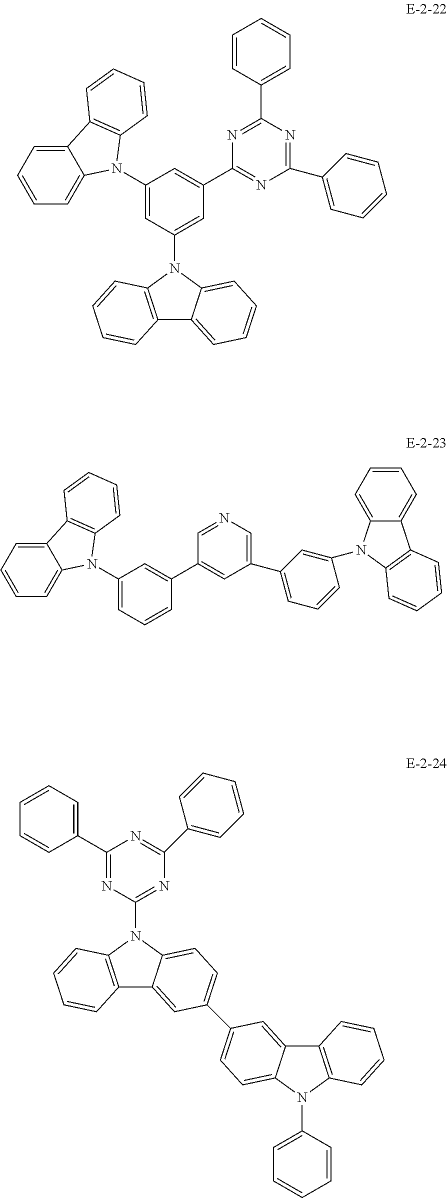 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a general material suitable in the art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (dichloromethane), 2,8-bis(diphenylphosphoryl)dibenzofuran (PPF), 4,4′,4″-tris(carbazol-9-yl)-triphenylamine (TCTA), or 1,3,5-tris(1-phenyl-1H-benzo[d]imidazol-2-yl)benzene (TPBi). However, the embodiment of the present disclosure is not limited thereto, and for example, tris(8-hydroxyquinolino) aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), and/or the like.

The emission layer EML may include a compound represented by Formula M-a or Formula M-b. The compound represented by Formula M-a or Formula M-b may be utilized as a phosphorescent dopant material.

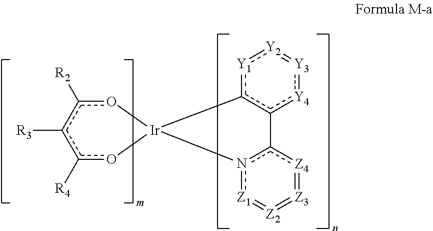

In Formula M-a, Y₁ to Y₄, and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring. In Formula M-a, m is 0 or 1, and n is 2 or 3. In Formula M-a, when m is 0, n is 3, and when m is 1, n is 2.

The compound represented by Formula M-a may be utilized as a phosphorescent dopant.

The compound represented by Formula M-a may be represented by any one selected from among compounds M-a1 to M-a25. However, the compounds M-a1 to M-a25 are presented as an example, and the compound represented by Formula M-a is not limited to those represented by the compounds M-a1 to M-a25.

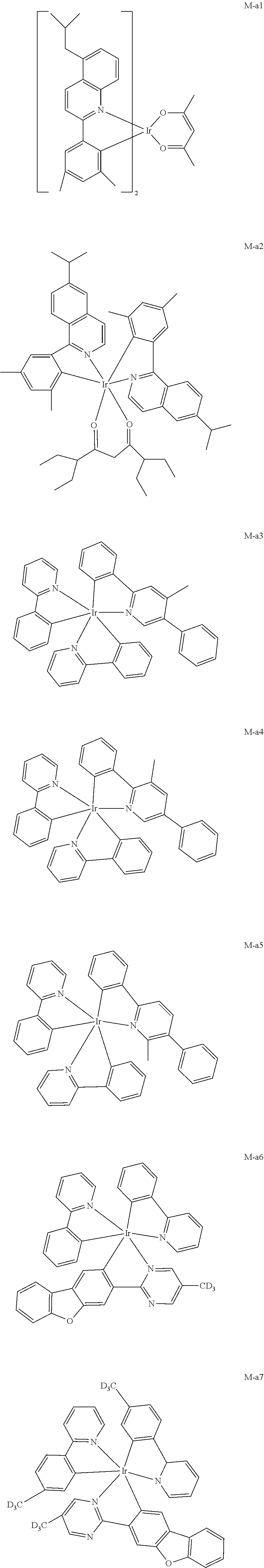

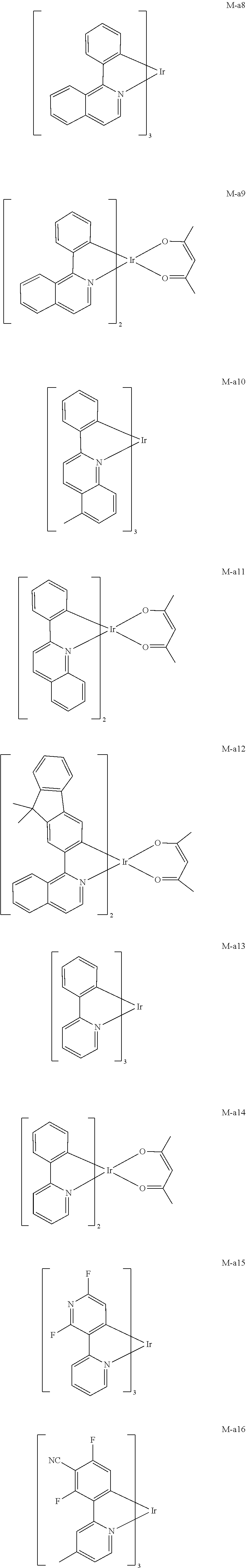

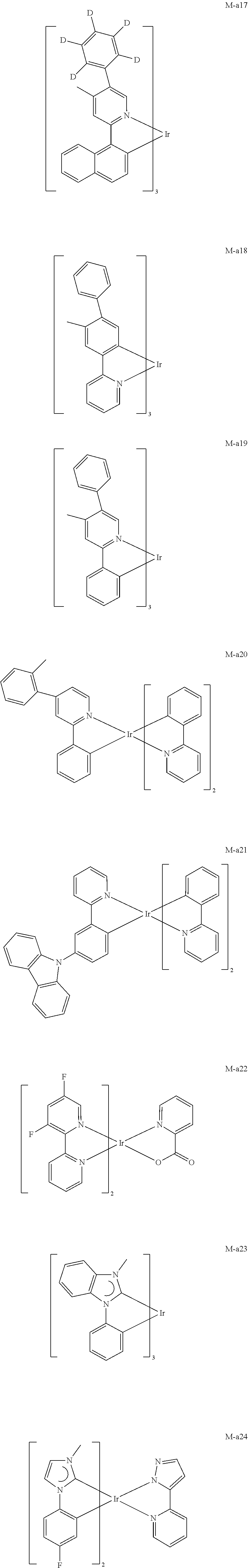

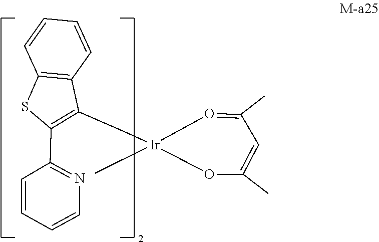

The compounds M-a1 and M-a2 may be utilized as a red dopant material, and the compounds M-a3 to M-a7 may be utilized as a green dopant material.

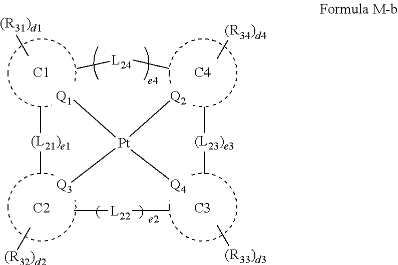

In Formula M-b, Q₁ to Q₄ may each independently be C or N, and C1 to C4 may each independently be a substituted or unsubstituted hydrocarbon ring having 5 to 30 ring-forming carbon atoms, or a substituted or unsubstituted heterocycle having 2 to 30 ring-forming carbon atoms. L₂₁ to L₂₄ may each independently be a direct linkage,

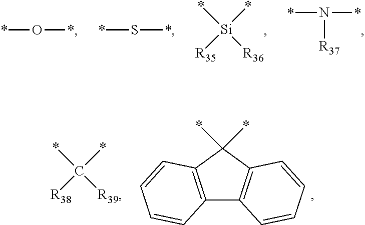

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, and d1 to d4 may each independently be an integer of 0 to 4.

The compound represented by Formula M-b may be utilized as a blue phosphorescent dopant or a green phosphorescent dopant.

The compound represented by Formula M-b may be represented by any one selected from among compounds M-b-1 to M-b-11. However, the compounds are presented as an example, and the compound represented by Formula M-b is not limited to those represented by the compounds M-b-1 to M-b-11.

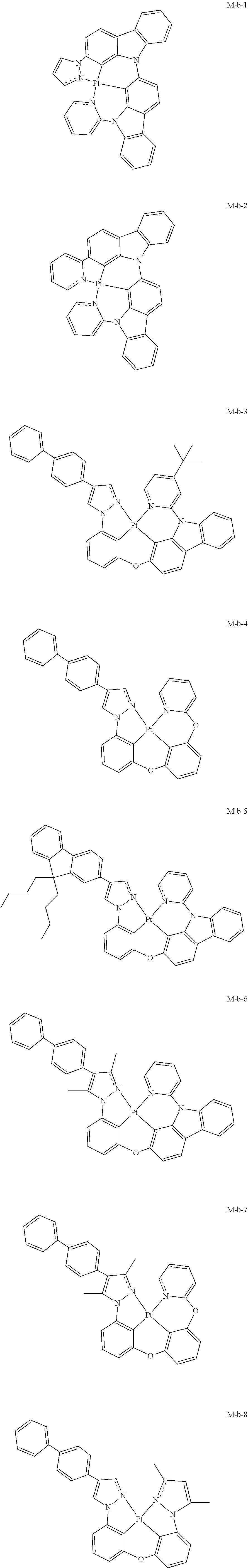

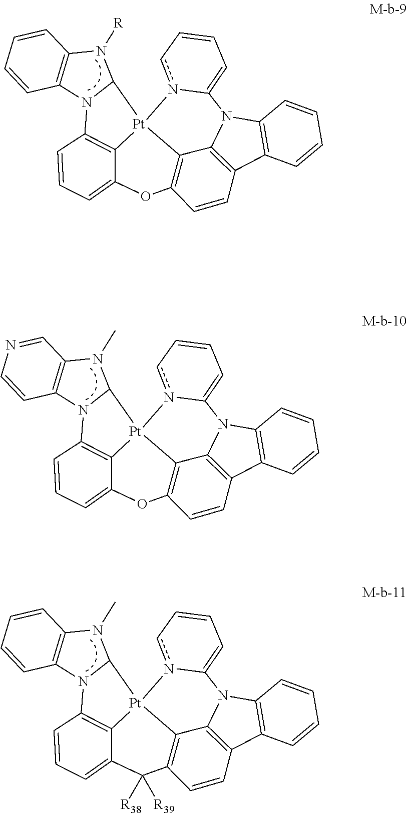

In the compounds M-b-9 and M-b-11, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may include a compound represented by any one selected from among Formulas F-a to F-c. In an embodiment, the compounds represented by Formulas F-a to F-c may be utilized as a fluorescence dopant material.

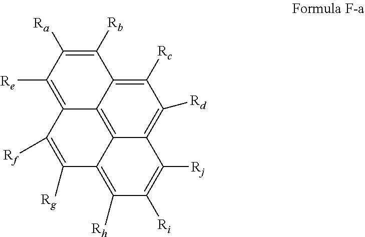

In Formula F-a, two selected from Rₐ to Rⱼ may each independently be substituted with *-NAr₁Ar₂. The others among Rₐ to Rⱼ which are not substituted with *-NAr₁Ar₂ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In *-NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

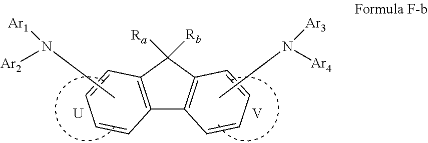

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms.

In Formula F-b, the number of rings represented by U and V may each independently be 0 or 1. For example, in Formula F-b, when the value of U or V is 1, one ring forms a fused ring in a portion indicated by U or V, and when the value of U or V is 0, then no ring indicated by U or V is present. For example, when the value of U is 0 and the value of V is 1, or when the value of U is 1 and the value of V is 0, a fused ring having a fluorene core of Formula F-b may be a cyclic compound having four rings. In some embodiments, when both (e.g., simultaneously) U and V are 0, the fused ring of Formula F-b may be a cyclic compound having three rings. In some embodiments, when both (e.g., simultaneously) U and V are 1, the fused ring having a fluorene core of Formula F-b may be a cyclic compound having five rings.

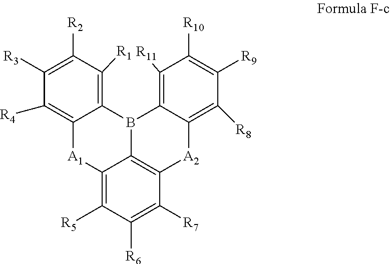

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring.

In Formula F-c, A₁ and A₂ may each independently be bonded to substituents of neighboring rings to form a fused ring. For example, A₁ and A₂ may each independently be NRₘ, and A₁ may be bonded to R₄ or R₅ to form a ring. In some embodiments, A₂ may be bonded to R₇ or R₈ to form a ring.

The emission layer EML may include, as a suitable dopant material, styryl derivatives (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and/or 4,4′-bis[2-(4-(N,N-diphenylamino)phenyl)vinyl]biphenyl (DPAVBi)), perylene and derivatives thereof (e.g., 2,5,8,11-tetra-t-butylperylene (TBP)), pyrene and derivatives thereof (e.g., 1,1-dipyrene, 1,4-dipyrenylbenzene, 1,4-bis(N,N-diphenylamino)pyrene), and/or the like. The emission layer EML may include a suitable phosphorescent dopant material. For example, as a phosphorescent dopant, a metal complex including an element selected from iridium (Ir), platinum (Pt), osmium (Os), gold (Au), titanium (Ti), zirconium (Zr), hafnium (Hf), europium (Eu), terbium (Tb), and/or thulium (Tm) may be utilized. For example, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), platinum octaethyl porphyrin (PtOEP), and/or the like may be utilized as a phosphorescent dopant. However, the embodiment of the present disclosure is not limited thereto.

The emission layer EML may include a quantum dot material. The core of a quantum dot may be selected from a Group II-VI compound, a Group III-VI compound, a Group I-III-VI compound, a Group III-V compound, a Group III-II-V compound, a Group IV-VI compound, a Group IV element, a Group IV compound, and/or a combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a mixture thereof.

The Group III-VI compound may include a binary compound such as In₂S₃ and/or In₂Se₃, a ternary compound such as InGaS₃ and/or InGaSe₃, or any combination thereof.

The Group I-III-VI compound may be selected from a ternary compound selected from the group consisting of AgInS, AgInS₂, CuInS, CuInS₂, AgGaS₂, CuGaS₂ CuGaO₂, AgGaO₂, AgAlO₂, and/or a mixture thereof, or a quaternary compound such as AgInGaS₂ and/or CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from the group consisting of GaAlNP, GaAlNAs, GaAlNSb, GaAlPAs, GaAlPSb, GalnNP, GalnNAs, GalnNSb, GalnPAs, GalnPSb, InAlNP, InAlNAs, InAlNSb, InAlPAs, InAlPSb, and a mixture thereof. In some embodiments, the Group III-V compound may further include a Group II metal. For example, InZnP, etc. may be selected as a Group III-II-V compound.

The Group IV-VI compound may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

In some embodiments, the binary compound, the ternary compound, and/or the quaternary compound may be present in particles having a substantially uniform concentration distribution, or may be present in substantially the same particles having a partially different concentration distribution. In some embodiments, a core/shell structure in which one quantum dot surrounds another quantum dot may be present. The core/shell structure may have a concentration gradient in which the concentration of an element present in the shell becomes lower towards the core.

In some embodiments, a quantum dot may have the core/shell structure including a core having nano-crystals, and a shell around (e.g., surrounding) the core, as described elsewhere herein. The shell of the quantum dot may be configured to be a protection layer to prevent or reduce the chemical deformation of the core and maintain or enhance the semiconductor properties thereof, and/or a charging layer to impart, maintain or enhance electrophoresis properties to, or of, the quantum dot. The shell may include a single layer or multiple layers. Non-limiting examples of the shell of the quantum dot include a metal or non-metal oxide, a semiconductor compound, and/or a combination thereof.

For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, and/or the like, or a ternary compound such as MgAl₂O₄, CoFe₂O₄, NiFe₂O₄, CoMn₂O₄, and/or the like, but the embodiment of the present disclosure is not limited thereto.

In some embodiments, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AIAs, AlP, AlSb, and/or the like, but the embodiment of the present disclosure is not limited thereto.

The quantum dot may have, in an emission wavelength spectrum, a full width of half maximum (FWHM) of about 45 nm or less, or about 40 nm or less, or about 30 nm or less, and in this range, the color purity or the color reproducibility may be improved. In some embodiments, light emitted by or through the quantum dot is emitted in all directions, and thus a wide viewing angle of a display device and/or light emitting element having the quantum dot as described herein may be enhanced or improved (e.g., the size or width of the viewing angle may be enhanced or increased).

In some embodiments, the form of a quantum dot is not particularly limited as long as it is a form commonly utilized in the art. In some embodiments, a quantum dot in the form of spherical, pyramidal, multi-arm, or cubic nanoparticles, nanotubes, nanowires, nanofibers, nanoplatelets, and/or the like may be utilized.

The quantum dot may determine or control the color of emitted light according to particle size thereof, and thus the quantum dot may have one or more suitable colors of emitted light such as blue, red, green, and/or the like.

In the light emitting element ED according to an embodiment shown in FIGS. 3 to 6, an electron transport region ETR is located or provided on the emission layer EML. The electron transport region ETR may include at least one of a hole blocking layer HBL, an electron transport layer ETL, or an electron injection layer EIL, but the embodiment of the present disclosure is not limited thereto.

The electron transport region ETR may have a single layer formed of a single material, a single layer formed of a plurality of different materials, and/or a multilayer structure having a plurality of layers formed of a plurality of different materials.

For example, the electron transport region ETR may have a single layer structure including (or made of) an electron injection layer EIL or an electron transport layer ETL, and/or may have a single layer structure including (or formed of) an electron injection material and/or an electron transport material. In some embodiments, the electron transport region ETR may have a single layer structure including (or formed of) a plurality of different materials, and/or may have a structure in which an electron transport layer ETL/electron injection layer EIL, or a hole blocking layer HBL/electron transport layer ETL/electron injection layer EIL are stacked in order from the emission layer EML, but is not limited thereto. The electron transport region ETR may have a thickness of, for example, about 1000 Å to about 1500 Å.

The electron transport region ETR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-2.

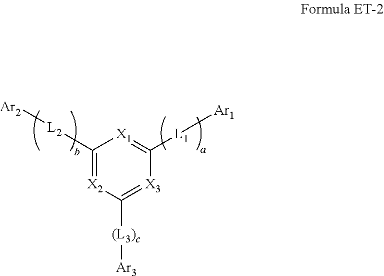

In Formula ET-2, at least one of X₁ to X₃ is N and the others are CRₐ. Each Rₐ may independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-2, a to c may each independently be an integer of 0 to 10. In Formula ET-2, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a to c are an integer of 2 or greater, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound, however, the embodiment of the present disclosure is not limited thereto. For example, the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), or a mixture thereof.

The electron transport region ETR may include at least one of compounds ET1 to ET36.

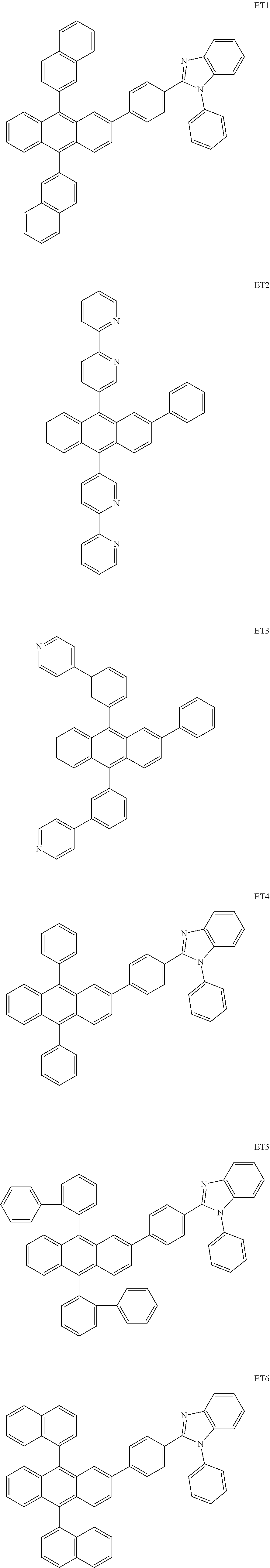

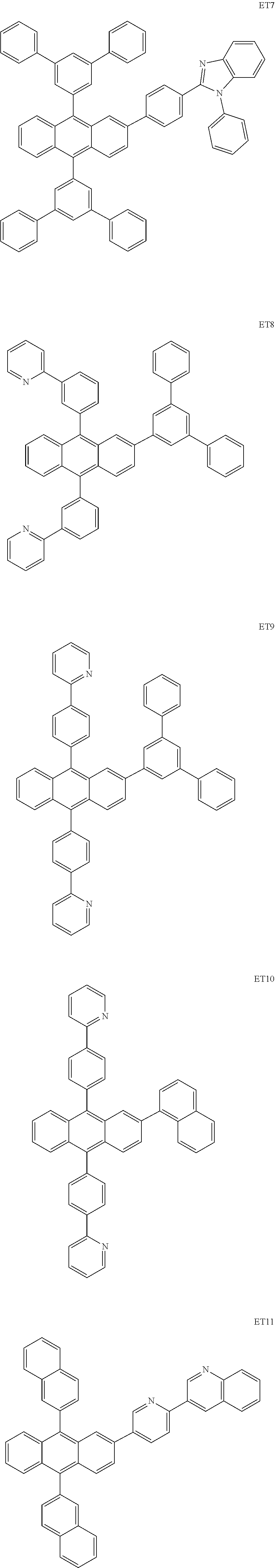 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1]

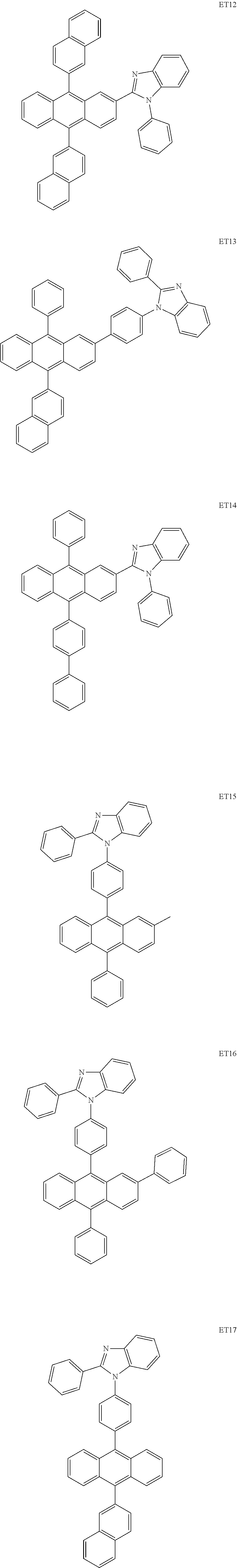 [SMILES: c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1]

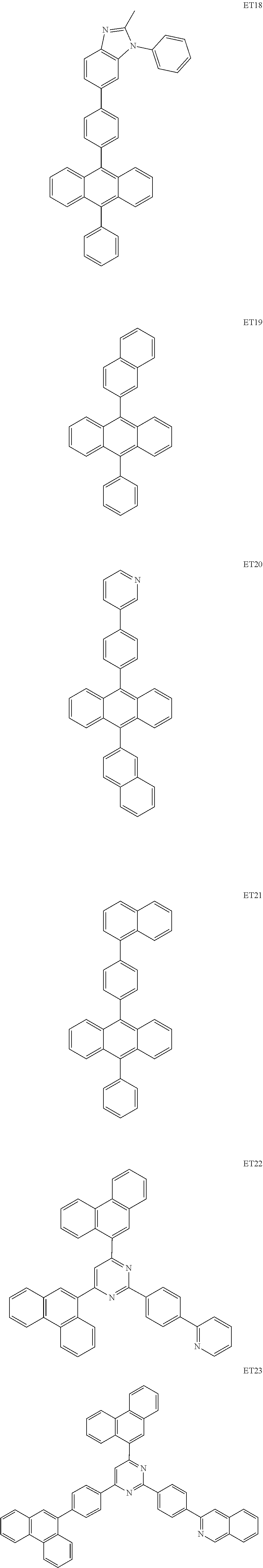

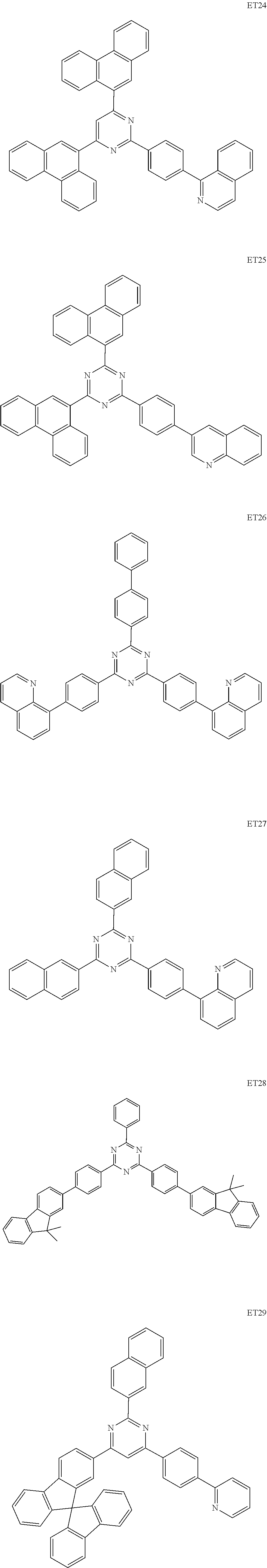

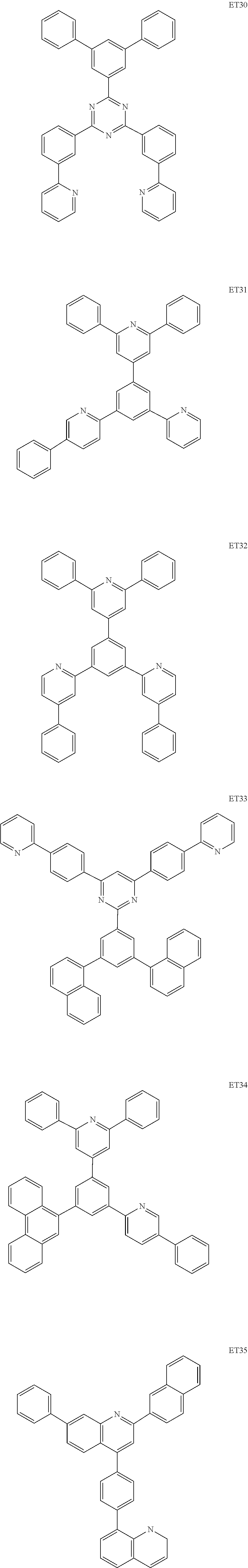

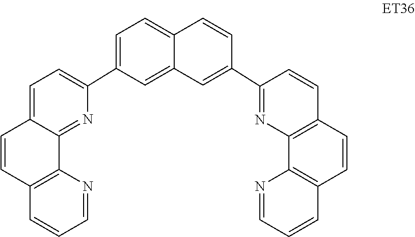 [SMILES: c1cnc2c(c1)ccc1ccc(-c3ccc4ccc(-c5ccc6ccc7cccnc7c6n5)cc4c3)nc12]

In some embodiments, the electron transport region ETR may include halogenated metals such as LiF, NaCl, CsF, RbCl, RbI, CuI, KI, and/or the like, lanthanide metals such as Yb and/or the like, or co-deposition materials of a halogenated metal and a lanthanide metal. For example, the electron transport region ETR may include KI:Yb, RbI:Yb, LiF:Yb, and/or the like, as a co-deposition material. In some embodiments, for the electron transport region ETR, a metal oxide such as Li₂O and BaO, or 8-hydroxyl-lithium quinolate (Liq), and/or the like may be utilized, but the embodiment of the present disclosure is limited thereto. The electron transport region ETR may also be formed of a mixture of materials including an electron transport material and an insulating organo-metal salt. In some embodiments, the electron transport region includes an organo-metal salt that may be a material having an energy band gap of about 4 eV or greater. The organo-metal salt may include, for example, a metal acetate, a metal benzoate, a metal acetoacetate, a metal acetylacetonate, a metal stearate, and/or the like.

The electron transport region ETR may further include, for example, at least one of 2,9-dimethyl-4,7-diphenyl-1,10-phenanthroline (BCP), diphenyl(4-(triphenylsilyl)phenyl)phosphine oxide (TSPO1), and/or 4,7-diphenyl-1,10-phenanthroline (Bphen) in addition to the materials described herein, but the embodiment of the present disclosure is not limited thereto.

The electron transport region ETR may include the compounds of the electron transport region described herein in at least one of the electron injection layer EIL, the electron transport layer ETL, or the hole blocking layer HBL.

When the electron transport region ETR includes the electron transport layer ETL, the electron transport layer ETL may have a thickness of about 100 Å to about 1000 Å, for example, about 150 Å to about 500 Å. When the thickness of the electron transport layer ETL is within these ranges, satisfactory electron transport properties may be obtained without (in absence of) a substantial increase in driving voltage. When the electron transport region ETR includes the electron injection layer EIL, the electron injection layer EIL may have a thickness of about 1 Å to about 100 Å, for example, about 3 Å to about 90 Å. When the thickness of the electron injection layer EIL is within these ranges, satisfactory electron injection properties may be obtained without (in absence of) a substantial increase in driving voltage.

The second electrode EL2 is located or provided on the electron transport region ETR. The second electrode EL2 may be a common electrode. The second electrode EL2 may be a cathode or an anode but the embodiment of the present disclosure is not limited thereto. For example, when the first electrode EL1 is an anode, the second electrode EL2 may be a cathode, and when the first electrode EL1 is a cathode, the second electrode EL2 may be an anode. The second electrode may include at least one selected from Ag, Mg, Cu, Al, Pt, Pd, Au, Ni, Nd, Ir, Cr, Li, Ca, LiF, Mo, Ti, W, In, Sn, and Zn, two or more compounds selected therefrom, two or more mixtures selected therefrom, or an oxide thereof.

The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), and/or the like.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound thereof, or a mixture thereof (e.g., AgMg, AgYb, or MgYb). In some embodiments, the second electrode EL2 may have a multilayer structure including a reflective film or a transflective film formed of the described materials, and a transparent conductive film formed of indium tin oxide (ITO), indium zinc oxide (IZO), zinc oxide (ZnO), indium tin zinc oxide (ITZO), and/or the like. For example, the second electrode EL2 may include the described metal materials, a combination of two or more metal materials selected from the described metal materials, or oxide(s) of the described metal materials.

Unlike the embodiments illustrated in FIGS. 3-6, the second electrode EL2 may be connected to or with an auxiliary electrode. When the second electrode EL2 is connected to or with the auxiliary electrode, the resistance of the second electrode EL2 may be optimized or decrease.

In some embodiments, a capping layer CPL may be further located or disposed on the second electrode EL2 of the light emitting element ED according to an embodiment. The capping layer CPL may include a multilayer or a single layer.

In an embodiment, the capping layer CPL may be an organic layer or an inorganic layer. For example, when the capping layer CPL includes an inorganic material, the inorganic material may include an alkali metal compound such as LiF, and/or an alkaline earth metal compound such as MgF₂, SiON, SiNx, SiOy, and/or the like.

For example, when the capping layer CPL includes an organic material, the organic material may include a-NPD, NPB, TPD, m-MTDATA, Alq₃ CuPc, N4, N4, N4′, N4′-tetra(biphenyl-4-yl) biphenyl-4,4′-diamine (TPD15), 4,4′,4″-tris(carbazol sol-9-yl)triphenylamine (TCTA), and/or the like, or may include epoxy resin(s) or acrylates such as methacrylate(s), however, the embodiment of the present disclosure is not limited thereto. For example, the capping layer CPL may include one or more compounds selected from P1 to P5.

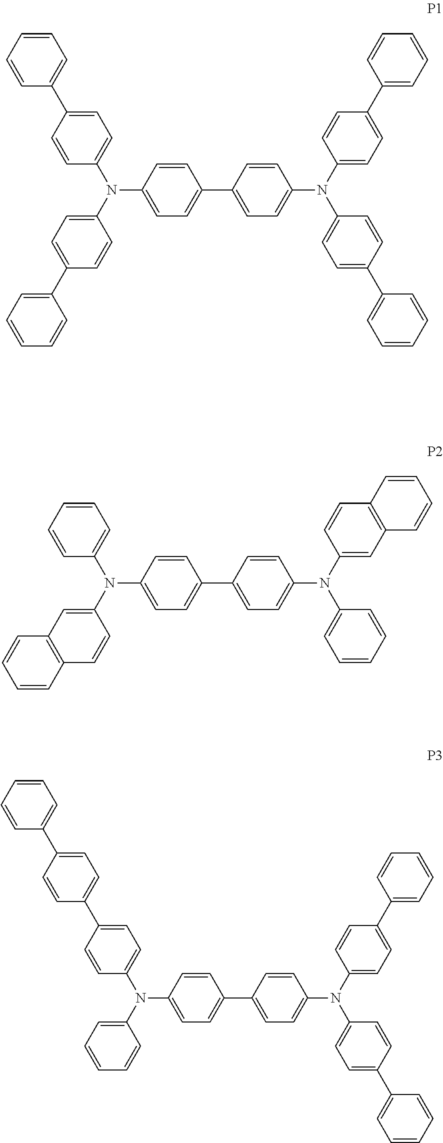 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

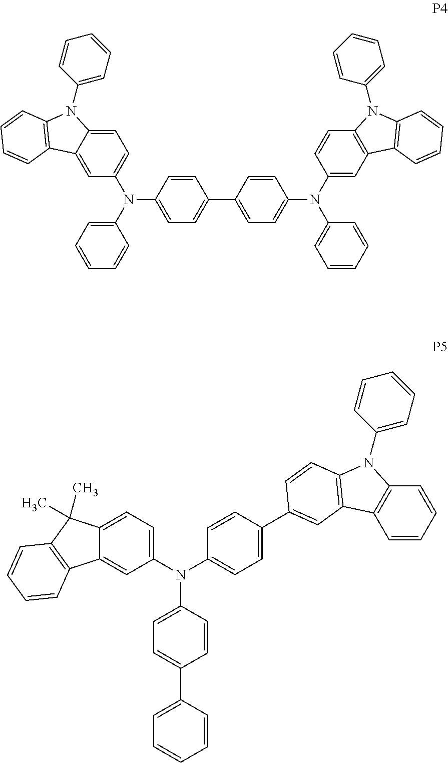 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the capping layer CPL may have a refractive index of about 1.6 or greater. For example, the capping layer CPL may have a refractive index of about 1.6 or greater in a wavelength range of about 550 nm to about 660 nm.

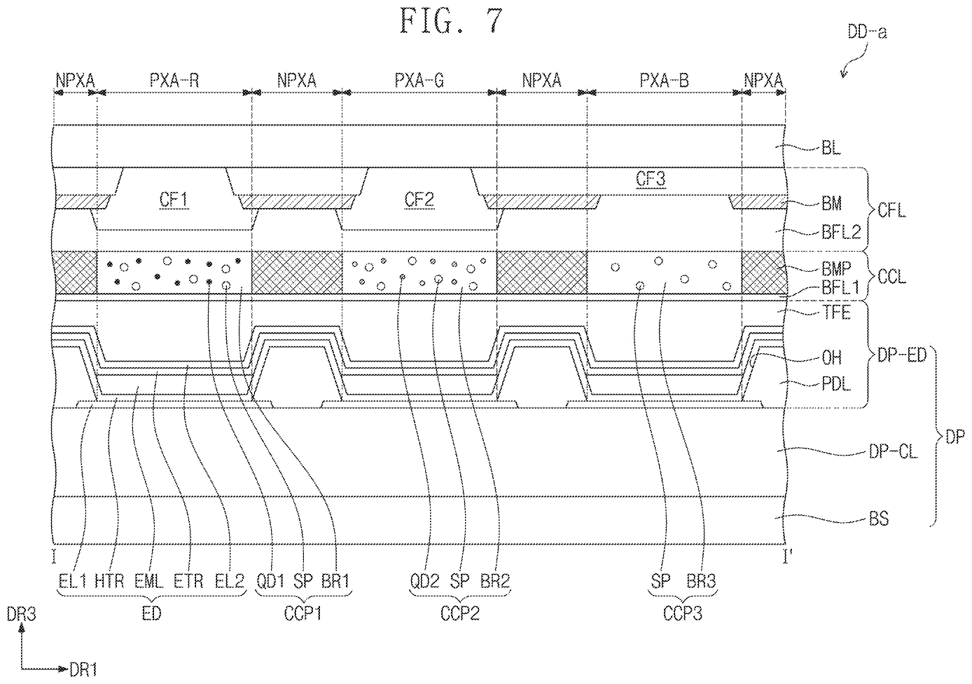

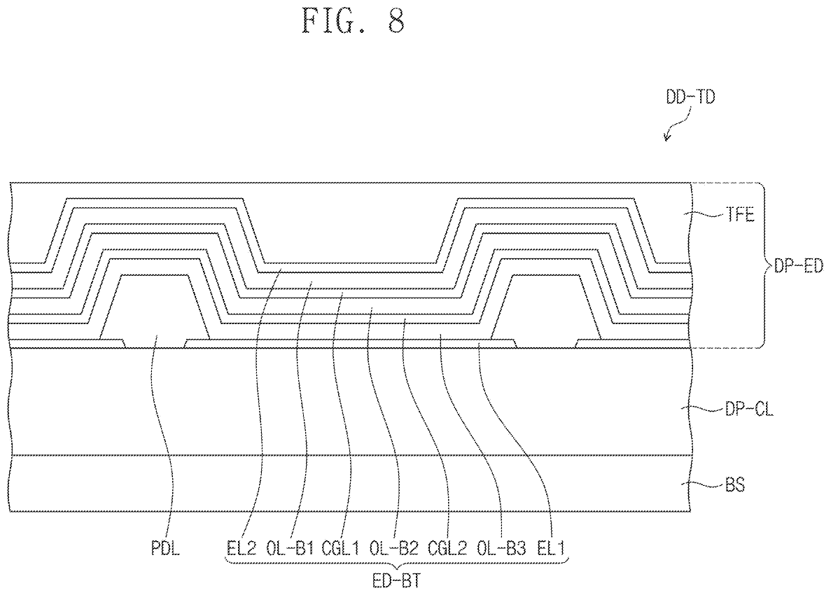

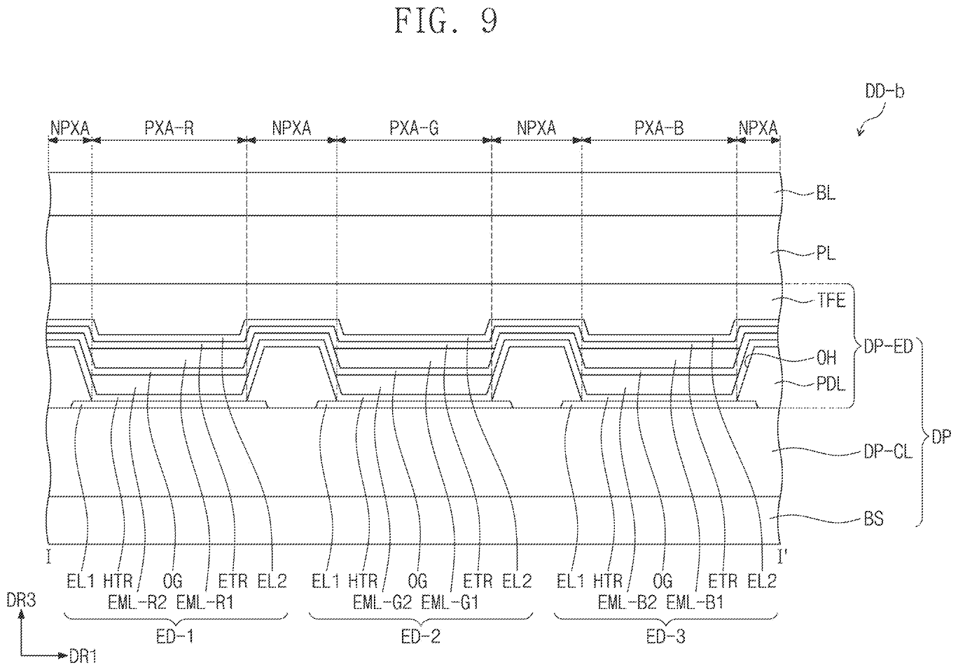

FIGS. 7 and 8 each are cross-sectional views of a display device according to an embodiment. Hereinafter, in the description of the display device according to an embodiment with reference to FIGS. 7 and 8, content (e.g., amount) overlapping the display device described with reference to FIGS. 1 to 6 will not be described again, instead the differences will be described (e.g., mainly described).

Referring to FIG. 7, a display device DD-a according to an embodiment may include a display panel DP having a display element layer DP-ED, a light control layer CCL located or disposed on the display panel DP, and a color filter layer CFL.

In an embodiment shown in FIG. 7, the display panel DP may include a base layer BS, a circuit layer DP-CL located or provided on the base layer BS, and a display element layer DP-ED, and the element layer DP-ED may include a light emitting element ED.

The light emitting element ED may include a first electrode EL1, a hole transport region HTR located or disposed on the first electrode EL1, an emission layer EML disposed on the hole transport region HTR, an electron transport region ETR located or disposed on the emission layer EML, and a second electrode EL2 located or disposed on the electron transport region ETR. In some embodiments, a structure of the light emitting element ED shown in FIG. 7 may be substantially the same as the structure of the light emitting element of FIGS. 3 to 6 as described.

Referring to FIG. 7, the emission layer EML may be located or disposed in the openings OH defined in the pixel defining films PDL. For example, the emission layer EML that is separated by the pixel defining films PDL and located or provided corresponding to each of light emitting regions PXA-R, PXA-G, and PXA-B may be configured to emit light in substantially the same wavelength ranges. In the display device DD-a of an embodiment, the emission layer EML may be configured to emit blue light. In some embodiments, unlike what is shown, in an embodiment, the emission layer EML may be located or provided as a common layer throughout the light emitting regions PXA-R, PXA-G, and PXA-B.

The light control layer CCL may be located or disposed on the display panel DP. The light control layer CCL may include a light converter. The light converter may be a quantum dot or a phosphor. The light converter may wavelength-convert the incident (e.g., incoming or provided) light and emit the wavelength-converted light. For example, the light control layer CCL may be a layer containing quantum dots or phosphors.

The light control layer CCL may include a plurality of light control units CCP1, CCP2, and CCP3. The light control units CCP1, CCP2, and CCP3 may be spaced from each other.

Referring to FIG. 7, a division pattern BMP may be located or disposed between the light control units CCP1, CCP2, and CCP3 spaced from each other, but the embodiment of the present disclosure is not limited thereto. In FIG. 7, the division pattern BMP is shown to non-overlap the light control units CCP1, CCP2, and CCP3, but edges of the light control units CCP1, CCP2, and CCP3 may overlap at least a portion of the division pattern BMP.

The light control layer CCL may include a first light control unit CCP1 including a first quantum dot QD1 for converting first color light provided from the light emitting element ED into second color light, a second light control unit CCP2 including a second quantum dot QD2 for converting the first color light into third color light, and a third light control unit CCP3 transmitting the first color light.

In an embodiment, the first light control unit CCP1 may emit or provide red light, which is the second color light, and the second light control unit CCP2 may emit or provide green light, which is the third color light. The third light control unit CCP3 may be configured to transmit and emit or provide blue light, which is the first color light emitted or provided from the light emitting element ED. For example, the first quantum dot QD1 may be a red quantum dot and the second quantum dot QD2 may be a green quantum dot. The same descriptions above may be applied to the quantum dots QD1 and QD2.

In some embodiments, the light control layer CCL may further include scatterers SP. The first light control unit CCP1 may include the first quantum dot QD1 and the scatterers SP, the second light control unit CCP2 may include the second quantum dot QD2 and the scatterers SP, and the third light control unit CCP3 may not include a (e.g., may exclude any) quantum dot but may include the scatterers SP.

The scatterers SP may be inorganic particles. For example, the scatterers SP may include at least one of TiO₂, ZnO, Al₂O₃, SiO₂, or hollow silica. The scatterers SP may include any one among TiO₂, ZnO, Al₂O₃, SiO₂, and hollow silica, or may be a mixture of two or more materials selected from TiO₂, ZnO, Al₂O₃, SiO₂, and hollow silica.

The first light control unit CCP1, the second light control unit CCP2, and the third light control unit CCP3 may each include base resins BR1, BR2, and BR3 for dispersing the quantum dots QD1 and QD2 and the scatterers SP. In an embodiment, the first light control unit CCP1 may include the first quantum dot QD1 and the scatterers SP dispersed in the first base resin BR1, the second light control unit CCP2 may include the second quantum dot QD2 and the scatterers SP dispersed in the second base resin BR2, and the third light control unit CCP3 may include the scatterers SP dispersed in the third base resin BR3. The base resins BR1, BR2, and BR3 are each a composition or medium in which the quantum dots QD1 and QD2 and the scatterers SP are dispersed, and may include (or be formed of) one or more suitable resin compositions, which may be generally referred to as a binder. For example, the base resins BR1, BR2, and BR3 may be an acrylic resin, a urethane-based resin, a silicone-based resin, an epoxy-based resin, and/or the like. The base resins BR1, BR2, and BR3 may be a transparent resin. In an embodiment, the first base resin BR1, the second base resin BR2, and the third base resin BR3 may each be the same as or different from each other.

The light control layer CCL may include a barrier layer BFL1. The barrier layer BFL1 may serve to prevent or reduce moisture and/or oxygen (hereinafter referred to as “moisture/oxygen”) from being introduced. The barrier layer BFL1 may be located or disposed on the light control units CCP1, CCP2, and CCP3 to prevent or reduce the light control units CCP1, CCP2, and CCP3 from being exposed to moisture/oxygen. In some embodiments, the barrier layer BFL1 may cover the light control units CCP1, CCP2, and CCP3. In some embodiments, a barrier layer BFL2 may be located or provided between the light control units CCP1, CCP2, and CCP3 and the color filter layer CFL.

The barrier layers BFL1 and BFL2 may include at least one inorganic layer. For example, the barrier layers BFL1 and BFL2 may include (or be formed of) an inorganic material. For example, the barrier layers BFL1 and BFL2 may include (or be formed of) silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, and/or a metal thin film in which light transmittance is secured, and/or the like. In some embodiments, the barrier layers BFL1 and BFL2 may further include an organic film. The barrier layers BFL1 and BFL2 may include (or be formed of) a single layer or a plurality of layers.

In the display device DD-a of an embodiment, the color filter layer CFL may be located or disposed on the light control layer CCL. For example, the color filter layer CFL may be directly located or disposed on the light control layer CCL. In this case, the barrier layer BFL2 may not be provided.

The color filter layer CFL may include a light blocking unit BM and filters CF1, CF2, and CF3. For example, the color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. The filters CF1, CF2, and CF3 may each include a polymer photosensitive resin, a pigment and/or a dye. The first filter CF1 may include a red pigment and/or a red dye, the second filter CF2 may include a green pigment and/or a green dye, and the third filter CF3 may include a blue pigment and/or a blue dye. In some embodiments, the embodiment of the present disclosure is not limited thereto, and the third filter CF3 may not include (e.g., may exclude) a (any) pigment and/or a (any) dye. The third filter CF3 may include a polymer photosensitive resin, but not include a (any) pigment and/or a (any) dye. The third filter CF3 may be transparent. The third filter CF3 may be formed of a transparent photosensitive resin.

In an embodiment, the first filter CF1 and the second filter CF2 may be yellow filters. The first filter CF1 and the second filter CF2 may not be separated and may be provided as a single body.

The light blocking portion BM may be a black matrix. The light blocking unit BM may include (or be formed of) an organic light blocking material and/or an inorganic light blocking material, both (e.g., simultaneously) including a black pigment and/or a black dye. The light blocking unit BM may prevent or reduce light leakage, and separate boundaries between the adjacent filters CF1, CF2, and CF3. In some embodiments, in an embodiment, the light blocking unit BM may include (or be formed of) a blue filter.

The first to third filters CF1, CF2, and CF3 may be located or disposed corresponding to the red light emitting region PXA-R, the green light emitting region PXA-G, and the blue light emitting region PXA-B, respectively.

The base substrate BL may be located or disposed on the color filter layer CFL. The base substrate BL may be a member configured as (or providing) a base surface on which the color filter layer CFL and the light control layer CCL are located or disposed. The base substrate BL may be a glass substrate, a metal substrate, a plastic substrate, and/or the like. However, the embodiment of the present disclosure is not limited thereto, and the base substrate BL may be an inorganic layer, an organic layer, or a composite material layer. In some embodiments, the base substrate BL may not be provided in an embodiment.

FIG. 8 is a cross-sectional view showing a portion of a display device according to an embodiment; FIG. 8 shows a cross-sectional view of a portion corresponding to the display panel DP of FIG. 7.

Referring to FIG. 8, a display device DD-TD of an embodiment includes a light emitting element ED-BT that may include a plurality of light emitting structures OL-B1, OL-B2, and OL-B3. The light emitting element ED-BT may include the first electrode EL1 and the second electrode EL2 facing each other, and the plurality of light emitting structures OL-B1, OL-B2, and OL-B3 provided by being sequentially stacked in a thickness direction between the first electrode EL1 and the second electrode EL2. The light emitting structures OL-B1, OL-B2, and OL-B3 each may include the emission layer EML (FIG. 7), and a hole transport region HTR and an electron transport region ETR located or disposed with the emission layer EML (FIG. 7) therebetween.

For example, the light emitting element ED-BT included in the display device DD-TD of an embodiment may be a light emitting element having a tandem structure including a plurality of emission layers.

In an embodiment shown in FIG. 8, light emitted from each of the light emitting structures OL-B1, OL-B2, and OL-B3 may all be blue light. However, the embodiment of the present disclosure is not limited thereto, and wavelength ranges of light emitted from each of the light emitting structures OL-B1, OL-B2, and OL-B3 may be different from each other. For example, the light emitting element ED-BT including the plurality of light emitting structures OL-B1, OL-B2, and OL-B3 emitting light in different wavelength ranges may be to emit white light.

A charge generation layer CGL may be disposed between neighboring light emitting structures OL-B1, OL-B2, and OL-B3. The charge generation layer CGL may include a p-type or kind charge generation layer (p-charge generation layer) and/or an n-type or kind charge generation layer (n-charge generation layer).

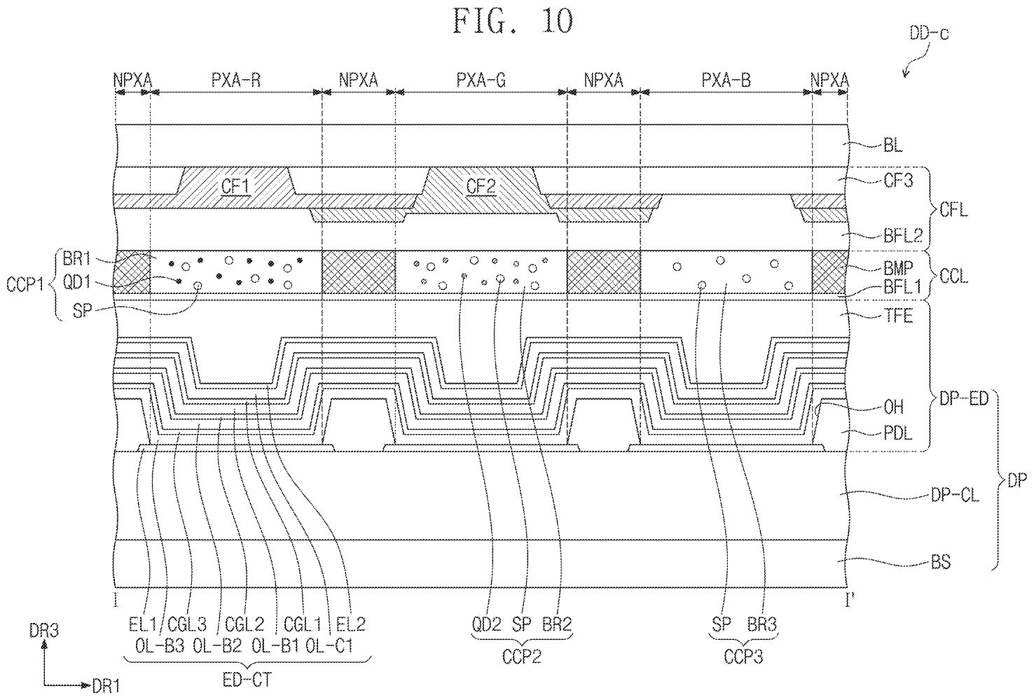

Referring to FIG. 9, a display device DD-b according to an embodiment may include light emitting elements ED-1, ED-2, and ED-3 in which two emission layers are stacked. Compared to the display device DD according to an embodiment shown in FIG. 2, the difference is that in an embodiment shown in FIG. 9, the first to third light emitting elements ED-1, ED-2, and ED-3 each include two emission layers stacked in a thickness direction. In each of the first to third light emitting elements ED-1, ED-2, and ED-3, the two emission layers may be configured to emit light in substantially the same wavelength range.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. A light emitting auxiliary portion OG may be located or disposed between the first red emission layer EML-R₁ and the second red emission layer EML-R₂, between the first green emission layer EML-G1 and the second green emission layer EML-G2, and/or between the first blue emission layer EML-B1 and the second blue emission layer EML-B2.

The light emitting auxiliary portion OG may include a single layer or multiple layers. The light emitting auxiliary portion OG may include a charge generation layer. To be more specific, the light emitting auxiliary portion OG may include an electron transport region, a charge generation layer, and a hole transport region that are sequentially stacked. The light emitting auxiliary portion OG may be provided as a common layer throughout the first to third light emitting elements ED-1, ED-2, and ED-3. However, the embodiment of the present disclosure is not limited thereto, and the light emitting auxiliary portion OG may be provided to be patterned inside the openings OH defined in the pixel defining films PDL.

The first red emission layer EML-R₁, the first green emission layer EML-G1, and/or the first blue emission layer EML-B1 may be disposed between the electron transport region ETR and the emission auxiliary portion OG. The second red emission layer EML-R₂, the second green emission layer EML-G2, and the second blue emission layer EML-B2 may be located or disposed between the emission auxiliary portion OG and the hole transport region HTR.

For example, the light emitting element ED-1 may include the first electrode EL1, the hole transport region HTR, the second red emission layer EML-R2, the emission auxiliary portion OG, the first red emission layer EML-R1, the electron transport region ETR, and the second electrode EL2, which are sequentially stacked. The second light emitting element ED-2 may include the first electrode EL1, the hole transport region HTR, the second green emission layer EML-G2, the emission auxiliary portion OG, the first green emission layer EML-G1, the electron transport region ETR, and the second electrode EL2, which are sequentially stacked. The third light emitting element ED-3 may include the first electrode EL1, the hole transport region HTR, the second blue emission layer EML-B2, the emission auxiliary portion OG, the first blue emission layer EML-B1, the electron transport region ETR, and the second electrode EL2, which are sequentially stacked.

In some embodiments, an optical auxiliary layer PL may be located or disposed on the display element layer DP-ED. The optical auxiliary layer PL may include a polarizing layer. The optical auxiliary layer PL may be located or disposed on the display panel DP to control reflected light in the display panel DP due to external light. Unlike what is shown, the optical auxiliary layer PL may not be provided in the display device according to an embodiment.

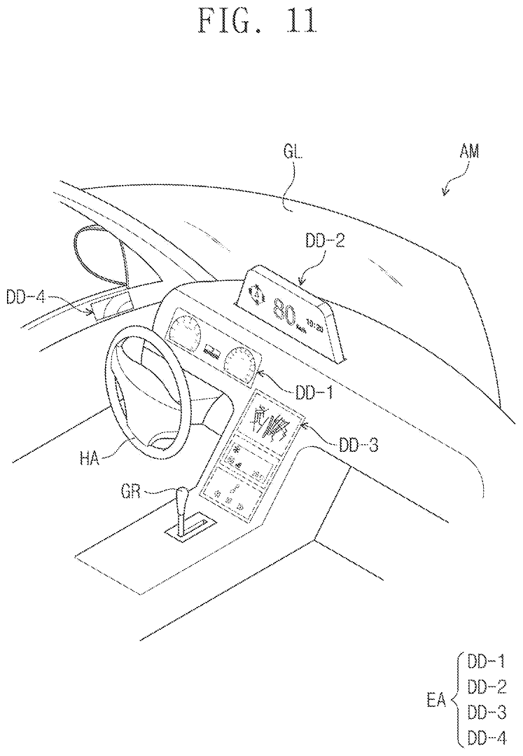

Referring to FIG. 10, the display device DD-c is configured to include four light emitting structures OL-B1, OL-B2, OL-B3, and OL-C1, unlike FIGS. 8 and 9. The light emitting element ED-CT may include the first electrode EL1 and the second electrode EL2 facing each other, and the first to fourth light emitting structures OL-B1, OL-B2, OL-B3, and OL-C1 sequentially stacked in a thickness direction between the first electrode EL1 and the second electrode EL2. Charge generation layers CGL1, CGL2, and CGL3 may be located or disposed between the first to fourth light emitting structures OL-B1, OL-B2, OL-B3, and OL-C1. Among the four light emitting structures, the first to third light emitting structures OL-B1, OL-B2, and OL-B3 may be configured to emit blue light, and the fourth light emitting structure OL-C1 may be configured to emit green light. However, the embodiment of the present disclosure is not limited thereto, and the first to fourth light emitting structures OL-B1, OL-B2, OL-B3, and OL-C1 may be configured to emit light having different wavelength ranges.

The charge generation layers CGL1, CGL2 and CGL3 located or disposed between the neighboring light emitting structures OL-B1, OL-B2, OL-B3, and OL-C1 may include a p-type or kind charge generation layer (p-charge generation layer) and/or an n-type or kind charge generation layer (n-charge generation layer).

FIG. 11 is a schematic perspective view of an electronic device including a display device according to an embodiment of the present disclosure. FIG. 11 shows an electronic device including a display device for a vehicle as an example.

Referring to FIG. 11, the electronic device EA of an embodiment may include display devices DD-1, DD-2, DD-3, and DD-4 for a vehicle AM. FIG. 11 shows first to fourth display devices DD-1, DD-2, DD-3, and DD-4 as display devices for the vehicle AM located or disposed inside the vehicle AM. In an embodiment, the first to fourth display devices DD-1, DD-2, DD-3, and DD-4 may be located or disposed in or on one or more suitable type or kind of transportation vehicles, such as bicycles, motorcycles, trains, ships (e.g. ocean ships), and airplanes. At least one of the first to fourth display devices DD-1, DD-2, DD-3, and DD-4 may have the same components as the display devices DD, DD-a, DD-b, and DD-c described with reference to FIGS. 1, 2, and 7 to 10.

In an embodiment, at least one of the first to fourth display devices DD-1, DD-2, DD-3, and DD-4 may include the light emitting element ED described with reference to FIGS. 3 to 6. The first to fourth display devices DD-1, DD-2, DD-3, and DD-4 may each independently include a plurality of light emitting elements ED, and each of the light emitting elements ED may include a first electrode EL1, a hole transport region HTL disposed above the first electrode EL1, an emission layer EML disposed above the hole transport region HTL, an electron transport region ETL disposed above the emission layer EML, and a second electrode EL2 disposed above the electron transport region ETL. In some embodiments, the emission layer EML may include a polycyclic compound of an embodiment represented by Formula 1 as described herein. Accordingly, the electronic device EA of an embodiment may exhibit enhanced or improved image quality.

Referring to FIG. 11, the vehicle AM may include a wheel HA and a gear GR for operating the vehicle AM, and have a front window GL disposed to face a driver.

The first display device DD-1 may be located or disposed in a first region overlapping the wheel HA. For example, the first display device DD-1 may be a digital cluster displaying first information of the vehicle AM. The first information may include a first scale indicating driving speed of the vehicle AM, a second scale indicating engine revolutions (i.e., revolutions per minute (RPM)), an image indicating fuel gauge, and/or the like. The first scale and the second scale may be displayed as digital images.

The second display device DD-2 may be located or disposed in a second region facing a driver seat and overlapping the front window WD. The driver seat may be a seat in which the wheel HA is disposed. For example, the second display device DD-2 may be a head up display HUD displaying second information of the vehicle AM. The second display device DD-2 may be optically transparent. The second information includes a digital number indicating a driving speed of the vehicle AM and may further include information such as current time.

The third display device DD-3 may be located or disposed in a third region adjacent to the gear GR. For example, the third display device DD-3 may be a center information display CID for a vehicle, which is located or disposed between a driver seat and a front passenger seat, and displays third information. The passenger seat may be a seat spaced apart from the driver seat with the gear GR therebetween. The third information may include information about road conditions (e.g., navigation information), music or radio play, dynamic video play, temperature inside the vehicle AM, and/or the like.

The fourth display device DD-4 may be located or disposed in a fourth region spaced apart from the wheel HA and the gear GR and adjacent to a side of the vehicle AM. For example, the fourth display device DD-4 may be a digital side mirror displaying fourth information. The fourth display device DD-4 may display images of conditions outside the vehicle AM, which are taken by a camera module disposed outside the vehicle AM. The fourth information may include images of conditions outside the vehicle AM.

The first to fourth information described above are presented as an example, and the first to fourth display devices DD-1, DD-2, DD-3, and DD-4 may further display information about inside or outside a vehicle. The first to fourth information may include different information. However, the embodiment of the present disclosure is not limited thereto, and some of the first to fourth information may include the same information.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ±30%, 20%, 10%, 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

The light emitting element, the display device and/or any other relevant devices or components according to embodiments of the present disclosure described herein may be implemented utilizing any suitable hardware, firmware (e.g., an application-specific integrated circuit), software, or a combination of software, firmware, and hardware. For example, the various components of the light emitting element and/or the display device may be formed on one integrated circuit (IC) chip or on separate IC chips. Further, the various components of the elements and/or devices may be implemented on a flexible printed circuit film, a tape carrier package (TCP), a printed circuit board (PCB), or formed on one substrate. Further, the various components of the elements and/or devices may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

In the present disclosure, when particles are spherical, “diameter” indicates an average particle diameter, and when the particles are non-spherical, the “diameter” indicates a major axis length. The diameter (or size) of the particles may be measured by particle size analysis, dynamic light scattering, scanning electron microscopy, and/or transmission electron microscope photography. When the size of the particles is measured utilizing a particle size analyzer, the average particle diameter (or size) may be referred to as D50. The term “D50” as utilized herein refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size. Particle size analysis may be performed with a HORIBA LA-950 laser particle size analyzer.

Hereinafter, with reference to Examples and Comparative Examples, a polycyclic compound and a light emitting element of an embodiment of the present disclosure will be specifically described. In some embodiments, Examples are shown only for the understanding of the present disclosure, and the scope of the present disclosure is not limited thereto.

### EXAMPLES

### 1. Synthesis of Polycyclic Compounds of Examples

A process of synthesizing polycyclic compounds according to an embodiment of the present disclosure will be described in more detail by presenting a process of synthesizing Compounds AO7, AO9, AO39, DO1, DS7, FO7, AO25, and AO19 as an example. In some embodiments, a process of synthesizing polycyclic compounds, which will be described hereinafter, is provided as an example, and thus a process of synthesizing compounds according to an embodiment of the present disclosure is not limited to Examples.

### (1) Synthesis of Compound AO7

Compound AO7 may be synthesized by Reaction Formula 1.

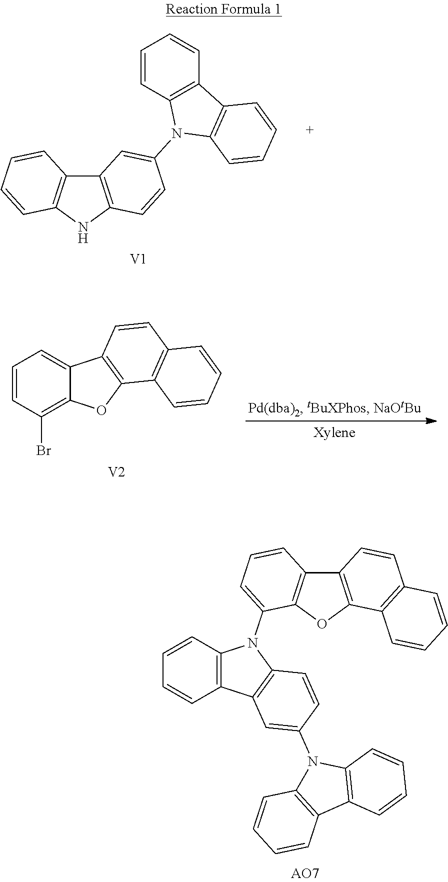

Xylene (200 mL) was added to compound V1 (10 mmol), compound V2 (10 mmol), NaOtBu (0.96 g, 10 mmol), and ^{t}BuXPhos (1 mmol) to form a mixture that was degassed. Bis(dibenzylideneacetone)palladium (0.5 mmol) was added in an argon atmosphere, and the mixture was heated and stirred at 130° C. for 12 hours. The reaction solution was cooled to room temperature, extracted with toluene, washed with H₂O and brine, and then dried through Na₂SO₄. The obtained solution was concentrated and purified through column chromatography to obtain compound AO7 (MS 548.19).

### (2) Synthesis of Compound AO9

Compound AO9 may be synthesized by Reaction Formula 2.

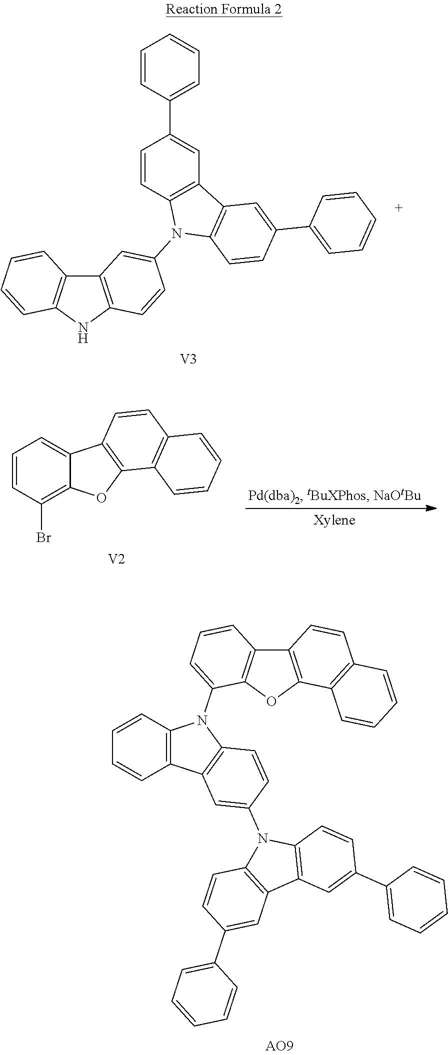

Xylene (200 mL) was added to compound V3 (10 mmol), compound V2 (10 mmol), NaO Bu (0.96 g, 10 mmol), and ^{t}BuXPhos (1 mmol) to form a mixture that was degassed. Bis(dibenzylideneacetone)palladium (0.5 mmol) was added in an argon atmosphere, and the mixture was heated and stirred at 130° C. for 12 hours. The reaction solution was cooled to room temperature, extracted with toluene, washed with H₂O and brine, and then dried through Na₂SO₄. The obtained solution was concentrated and purified through column chromatography to obtain compound AO9 (MS 700.25).

### (3) Synthesis of Compound AO39

Compound AO39 may be synthesized by Reaction Formula 3.

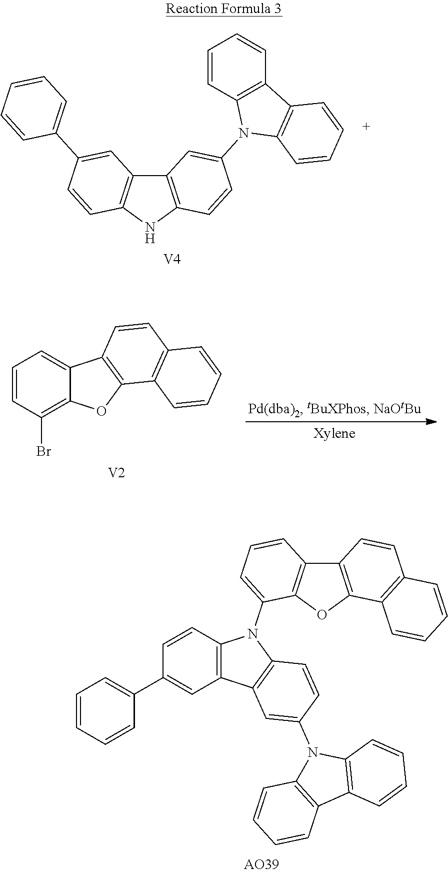

Xylene (200 mL) was added to compound V4 (10 mmol), compound V2 (10 mmol), NaO^{t}Bu (0.96 g, 10 mmol), and ^{t}BuXPhos (1 mmol) to form a mixture that was degassed. Bis(dibenzylideneacetone)palladium (0.5 mmol) was added in an argon atmosphere, and the mixture was heated and stirred at 130° C. for 12 hours. The reaction solution was cooled to room temperature, extracted with toluene, washed with H₂O and brine, and then dried through Na₂SO₄. The obtained solution was concentrated and purified through column chromatography to obtain compound AO39 (MS 624.22).

### (4) Synthesis of Compound DO1

Compound DO1 may be synthesized by Reaction Formula 4.

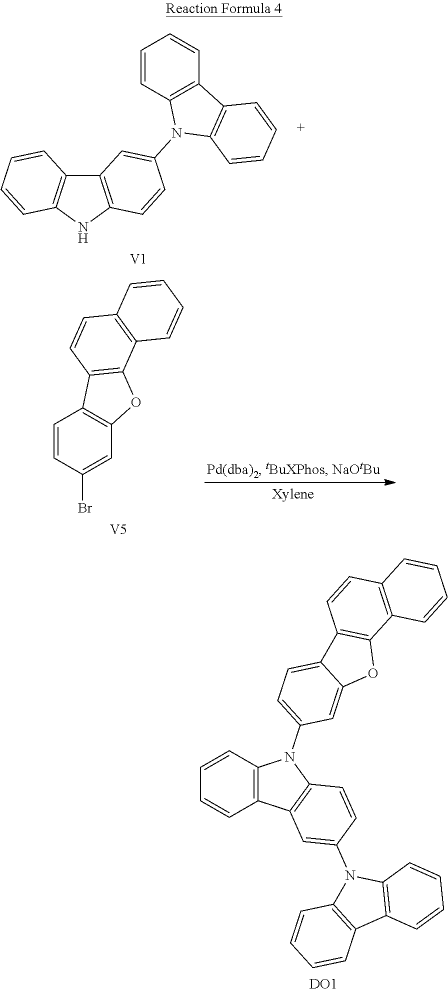

Xylene (200 mL) was added to compound V1 (10 mmol), compound V5 (10 mmol), NaO Bu (0.96 g, 10 mmol), and ^{t}BuXPhos (1 mmol) to form a mixture that was degassed. Bis(dibenzylideneacetone)palladium (0.5 mmol) was added in an argon atmosphere, and the mixture was heated and stirred at 130° C. for 12 hours. The reaction solution was cooled to room temperature, extracted with toluene, washed with H₂O and brine, and then dried through Na₂SO₄. The obtained solution was concentrated and purified through column chromatography to obtain compound DO1 (MS 548.19).

### (5) Synthesis of Compound DS7

Compound DS7 may be synthesized by Reaction Formula 5.

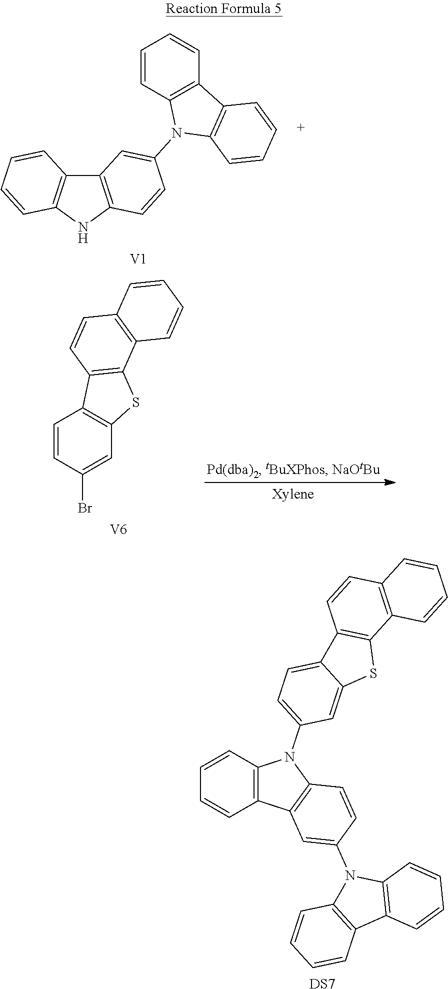

Xylene (200 mL) was added to compound V1 (10 mmol), compound V6 (10 mmol), NaOtBu (0.96 g, 10 mmol), and ^{t}BuXPhos (1 mmol) to form a mixture that was degassed. Bis(dibenzylideneacetone)palladium (0.5 mmol) was added in an argon atmosphere, and the mixture was heated and stirred at 130° C. for 12 hours. The reaction solution was cooled to room temperature, extracted with toluene, washed with H₂O and brine, and then dried through Na₂SO₄. The obtained solution was concentrated and purified through column chromatography to obtain compound DS7 (MS 564.17).

### (6) Synthesis of Compound FO7

Compound FO7 may be synthesized by Reaction Formula 6.

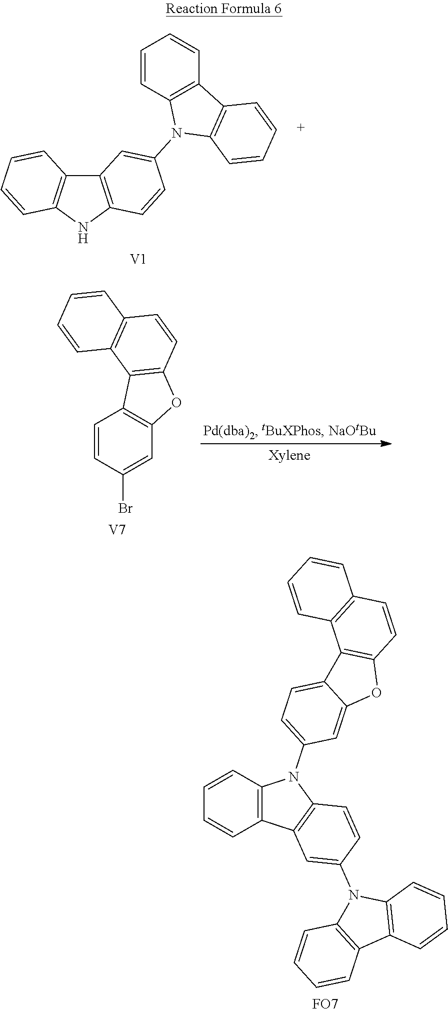

Xylene (200 mL) was added to compound V1 (10 mmol), compound V7 (10 mmol), NaOtBu (0.96 g, 10 mmol), and ^{t}BuXPhos (1 mmol) to form a mixture that was degassed. Bis(dibenzylideneacetone)palladium (0.5 mmol) was added in an argon atmosphere, and the mixture was heated and stirred at 130° C. for 12 hours. The reaction solution was cooled to room temperature, extracted with toluene, washed with H₂O and brine, and then dried through Na₂SO₄. The obtained solution was concentrated and purified through column chromatography to obtain compound FO7 (MS 548.19).

### (7) Synthesis of Compound AO25

Compound AO25 may be synthesized by Reaction Formula 7.

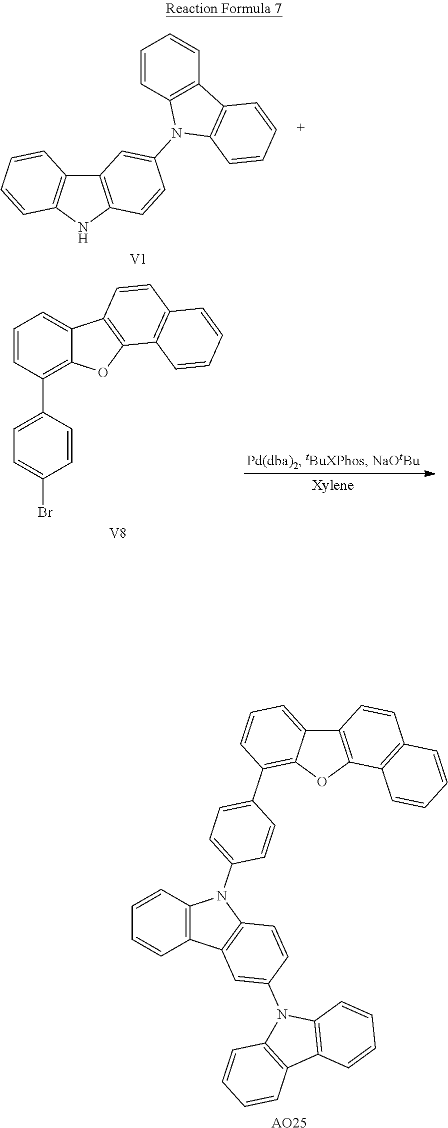

Xylene (200 mL) was added to compound V1 (10 mmol), compound V8 (10 mmol), NaO^{t}Bu (0.96 g, 10 mmol), and ^{t}BuXPhos (1 mmol) to form a mixture that was degassed. Bis(dibenzylideneacetone)palladium (0.5 mmol) was added in an argon atmosphere, and the mixture was heated and stirred at 130° C. for 12 hours. The reaction solution was cooled to room temperature, extracted with toluene, washed with H₂O and brine, and then dried through Na₂SO₄. The obtained solution was concentrated and purified through column chromatography to obtain compound AO25 (MS 624.22).

### (8) Synthesis of Compound AO19

Compound AO19 may be synthesized by Reaction Formula 8.

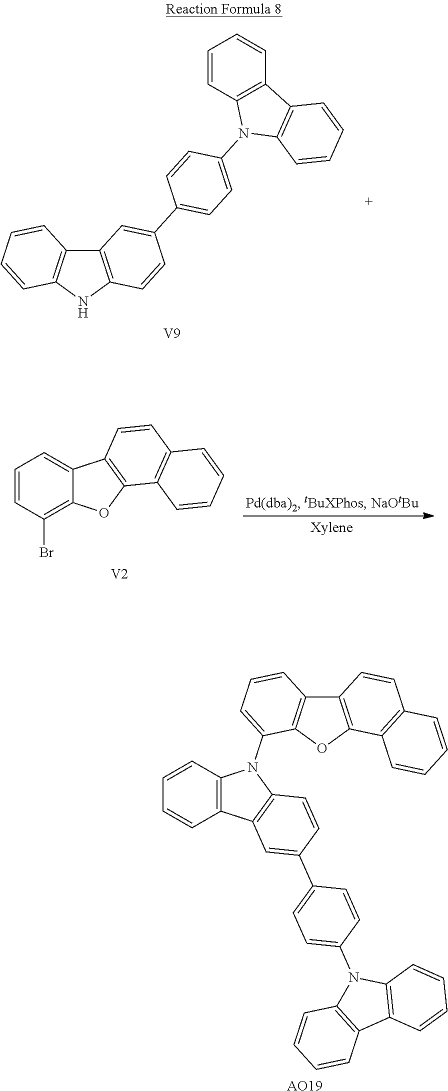

Xylene (200 mL) was added to compound V9 (10 mmol), compound V2 (10 mmol), NaO^{t}Bu (0.96 g, 10 mmol), and ^{t}BuXPhos (1 mmol) to form a mixture that was degassed. Bis(dibenzylideneacetone)palladium (0.5 mmol) was added in an argon atmosphere, and the mixture was heated and stirred at 130° C. for 12 hours. The reaction solution was cooled to room temperature, extracted with toluene, washed with H₂O and brine, and then dried through Na₂SO₄. The obtained solution was concentrated and purified through column chromatography to obtain compound AO7 (MS 624.22).

### 2. Preparation and Evaluation of Light Emitting Elements

### (1) Preparation of Light Emitting Elements

Light emitting elements including polycyclic compounds according to an embodiment or the Comparative Example compounds in a hole transport layer were prepared.

Light emitting elements of Examples 1 to 8 (LE-1 to LE-8) were prepared utilizing polycyclic compounds AO7, AO9, AO39, DO1, DS7, FO7, AO25, and AO19 as a material of the hole transport layer.

Light emitting elements of Comparative Examples 1 to 6 (CE-1 to CE-6) were prepared utilizing Comparative Example compounds R1 to R6 as a material of the hole transport layer.

As an anode, an ITO glass substrate (Corning, a thickness of 15 ohm per square centimeter (Ω/cm²)) was cut to a size of 50 millimeter (mm)×50 mm×0.7 mm, subjected to ultrasonic cleaning utilizing isopropyl alcohol and pure water for 5 minutes respectively and ultraviolet irradiation for 30 minutes, and then exposed to ozone for cleaning to form the glass substrate in a vacuum deposition apparatus.

To prepare the light emitting element, 2-TNATA was vacuum-deposited to have a thickness of 60 nm as a hole injection layer on the glass substrate. A hole transport layer was produced by vacuum-deposition of an Example compound or a Comparative Example compound to have a thickness of 30 nm. Co-deposition of 9,10-di(naphthalen-2-yl)anthracene (hereinafter, ADN) as a suitable blue fluorescent host and 2,5,8,11-tetra-tert-butylperylene (hereinafter, TBP) as a suitable blue fluorescent dopant at a weight ratio of 97:3 on an upper portion of the hole transport layer formed an emission layer having a thickness of 25 nanometer (nm). As an electron transport layer, Alq₃ was deposited to have a thickness of 25 nm on an upper portion of the emission layer. Then LiF, which is a halogenated alkali metal, was deposited to have a thickness of 1 nm as an electron injection layer, followed by vacuum-deposition of Al to have a thickness of 100 nm to form a LiF/Al electrode.

### Materials Utilized for Element Preparation

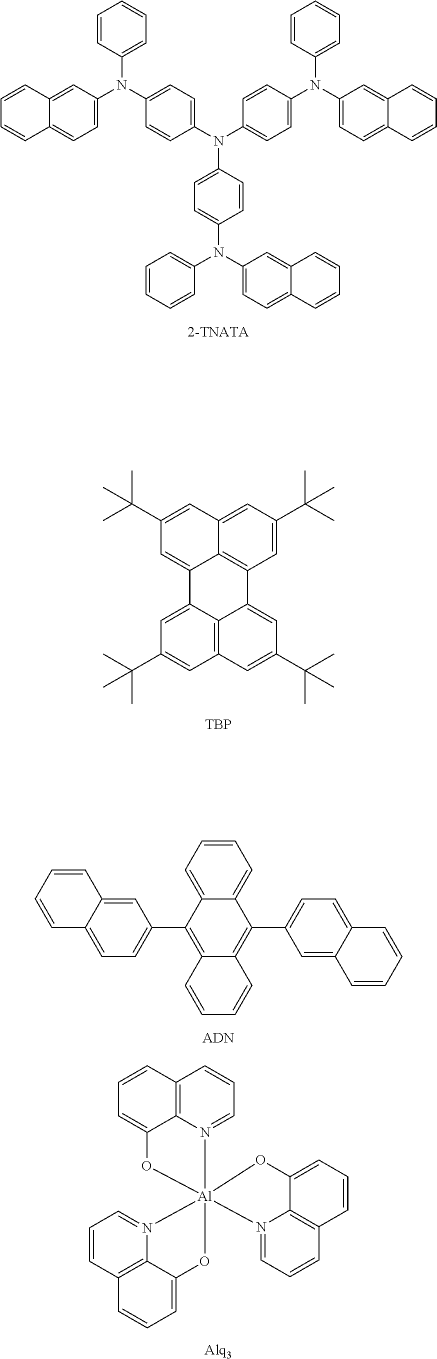

### Example Compounds

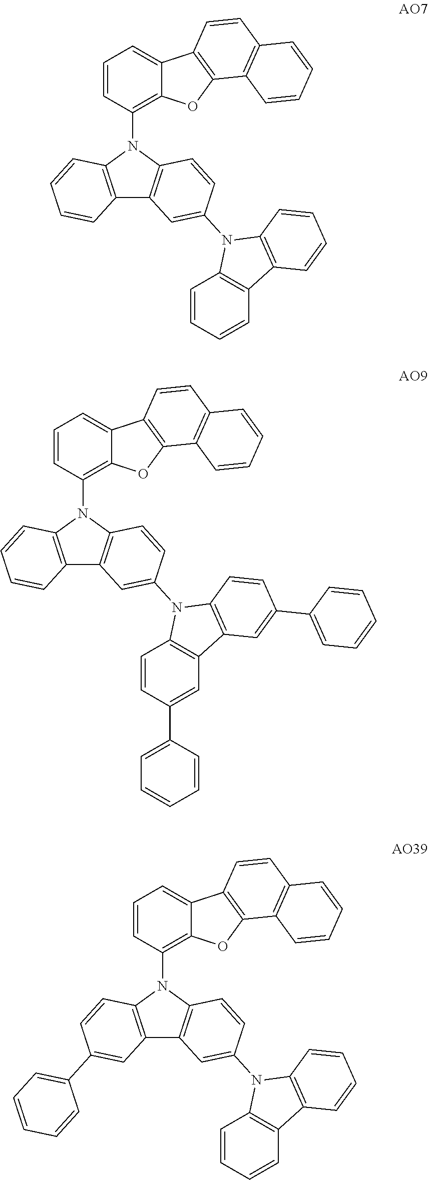 [SMILES: c1ccc2c(c1)ccc1c3cccc(-n4c5ccccc5c5cc(-n6c7ccccc7c7ccccc76)ccc54)c3oc21 ; c1ccc(-c2ccc3c(c2)c2cc(-c4ccccc4)ccc2n3-c2ccc3c(c2)c2ccccc2n3-c2cccc3c2oc2c4ccccc4ccc32)cc1 ; c1ccc(-c2ccc3c(c2)c2cc(-n4c5ccccc5c5ccccc54)ccc2n3-c2cccc3c2oc2c4ccccc4ccc32)cc1]

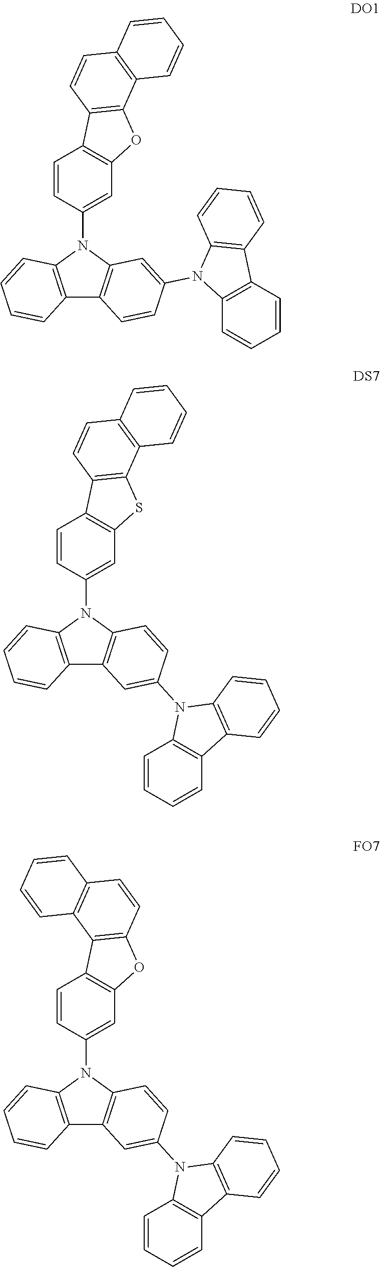 [SMILES: c1ccc2c(c1)ccc1c3ccc(-n4c5ccccc5c5ccc(-n6c7ccccc7c7ccccc76)cc54)cc3oc21 ; c1ccc2c(c1)ccc1c3ccc(-n4c5ccccc5c5cc(-n6c7ccccc7c7ccccc76)ccc54)cc3sc21 ; c1ccc2c(c1)ccc1oc3cc(-n4c5ccccc5c5cc(-n6c7ccccc7c7ccccc76)ccc54)ccc3c12]

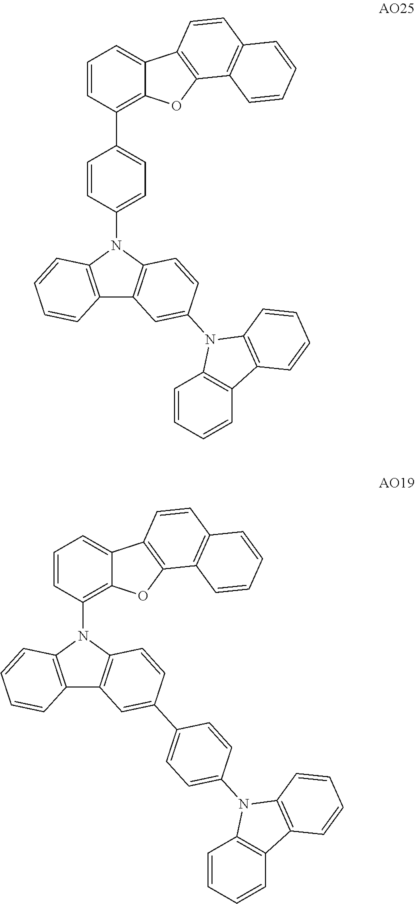 [SMILES: c1ccc2c(c1)ccc1c3cccc(-c4ccc(-n5c6ccccc6c6cc(-n7c8ccccc8c8ccccc87)ccc65)cc4)c3oc21 ; c1ccc2c(c1)ccc1c3cccc(-n4c5ccccc5c5cc(-c6ccc(-n7c8ccccc8c8ccccc87)cc6)ccc54)c3oc21]

### Comparative Example Compounds

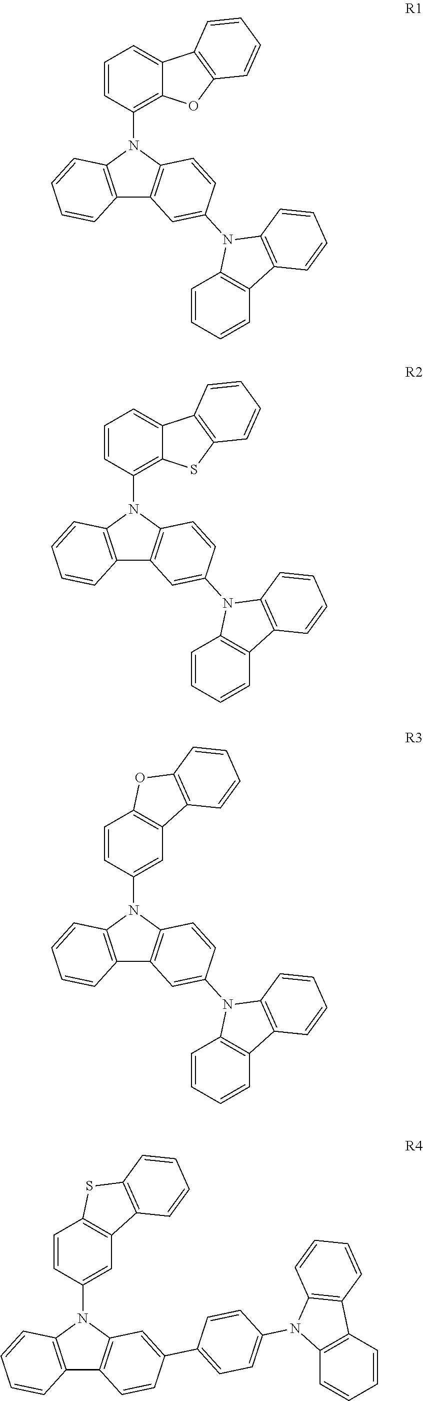 [SMILES: c1ccc2c(c1)oc1c(-n3c4ccccc4c4cc(-n5c6ccccc6c6ccccc65)ccc43)cccc12 ; c1ccc2c(c1)sc1c(-n3c4ccccc4c4cc(-n5c6ccccc6c6ccccc65)ccc43)cccc12 ; c1ccc2c(c1)oc1ccc(-n3c4ccccc4c4cc(-n5c6ccccc6c6ccccc65)ccc43)cc12 ; c1ccc2c(c1)sc1ccc(-n3c4ccccc4c4ccc(-c5ccc(-n6c7ccccc7c7ccccc76)cc5)cc43)cc12]

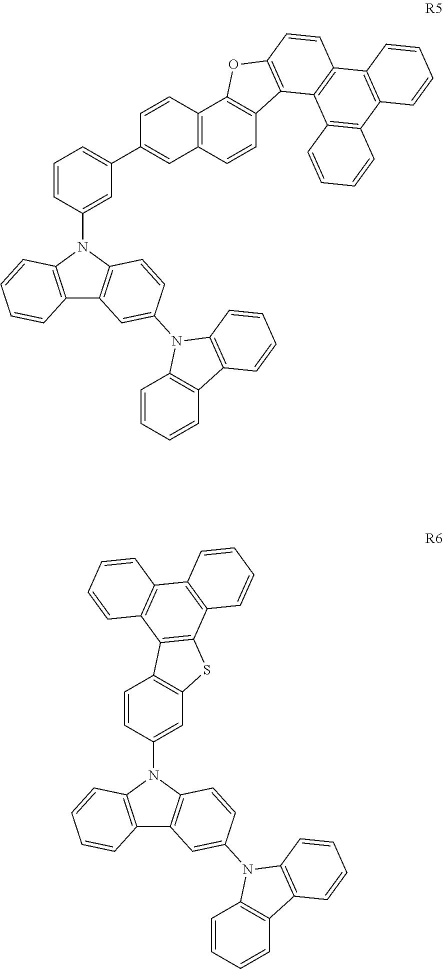 [SMILES: c1cc(-c2ccc3c(ccc4c3oc3ccc5c6ccccc6c6ccccc6c5c34)c2)cc(-n2c3ccccc3c3cc(-n4c5ccccc5c5ccccc54)ccc32)c1 ; c1ccc2c(c1)c1ccccc1c1c3ccc(-n4c5ccccc5c5cc(-n6c7ccccc7c7ccccc76)ccc54)cc3sc21]

### (2) Property Evaluation of Light Emitting Elements

Table 2 shows the evaluation of power efficiency in the light emitting elements of Examples 1-8 (LE-1 to LE-8) and Comparative Examples 1-6 (CE-1 to CE-6). The power efficiency was evaluated with respect to a current density of 10 milliamps per square centimeter (mA/cm²).

TABLE 2

| Light emitting elements | | | Light emitting elements | | |
|---|---|---|---|---|---|
| Examples 1-8 | | | Comparative Examples 1-6 | | |
| | Hole | Power | Compar- | Hole | Power |
| | transport | effi- | ative | transport | effi- |
| Example | layer Com- | ciency | example | layer Com- | ciency |
| ID | pound ID | (%) | ID | pound ID | (%) |
| LE-1 | AO7 | 112 | CE-1 | R1 | 100% |
| LE-2 | AO9 | 117 | CE-2 | R2 | 102% |
| LE-3 | AO39 | 114 | CE-3 | R3 | 103% |
| LE-4 | DO1 | 111 | CE-4 | R4 | 99% |
| LE-5 | DS7 | 112 | CE-5 | R5 | 97% |
| LE-6 | FO7 | 116 | CE-6 | R6 | 98% |
| LE-7 | AO25 | 114 | | | |
| LE-8 | AO19 | 117 | | | |

Referring to Table 2, it is seen that, compared to the light emitting elements of Comparative Examples 1 to 6 (CE-1 to CE-6), the light emitting elements of Examples 1 to 8 (LE-1 to LE-8) have improved power efficiency. Compounds AO7, AO9, AO39, DO1, DS7, FO7, AO25, and AO19, which are polycyclic compounds of an embodiment, include two carbazole groups and a benzonaphthalo heteroaryl group. Comparative Example compounds R1 to R6 include two carbazole groups, but do not include a benzonaphthalo heteroaryl group. Accordingly, it is seen that light emitting elements have improved power efficiency when including a hole transport layer including the polycyclic compound containing two carbazole groups and a benzonaphthalo heteroaryl group. This is attributed to an excellent or suitable hole transport ability of the polycyclic compound including two carbazole groups and a benzonaphthalo heteroaryl group.

Referring to Examples 1 to 3 (LE-1 to LE-3), even when the carbazole groups include a substituent in the polycyclic compound of an embodiment, it is seen that the light emitting elements have improved power efficiency. In some embodiments, referring to Examples 4 to 6 (LE-4 to LE-6), it is seen that the light emitting elements have improved power efficiency even when the position where the carbazole groups are substituted on the benzonaphthalo heteroaryl group is different in the polycyclic compound of an embodiment. Referring to Example 7 (LE-7), it is seen that the light emitting elements have improved power efficiency even when the benzonaphthalo heteroaryl group is bonded to the carbazole groups through a phenylene group in the polycyclic compound of an embodiment. Referring to Example 8 (LE-8), it is seen that the light emitting elements have improved power efficiency even when any one carbazole group is bonded to another carbazole group through a phenylene group in the polycyclic compound of an embodiment.

A light emitting element of an embodiment includes a first electrode, a second electrode disposed on the first electrode, and a hole transport region disposed between the first electrode and the second electrode. The hole transport region includes a polycyclic compound of an embodiment. The polycyclic compound of an embodiment includes two carbazole groups and a benzonaphthalo heteroaryl group, and thus has an excellent or suitable hole transport ability. Accordingly, a light emitting element of an embodiment including the polycyclic compound of an embodiment exhibits excellent or suitable power efficiency.

A light emitting element of an embodiment includes a polycyclic compound of an embodiment, and may thus exhibit high power efficiency.

A polycyclic compound of an embodiment may contribute to increasing power efficiency of a light emitting element.

Although the embodiments of the present disclosure have been described with reference to a preferred embodiment of the present disclosure, it will be understood by those skilled in the art that the present disclosure should not be limited to these preferred embodiments but one or more suitable changes and modifications can be made by those skilled in the art without departing from the spirit and scope of the present disclosure as set forth in the following claims and equivalents thereof.

Accordingly, the technical scope of the present disclosure is not to be limited to the contents set forth in the detailed description of the specification, but is intended to be defined by the appended claims.

What is claimed is:

1. A light emitting element comprising:
a first electrode;
a second electrode on the first electrode;
an emission layer between the first electrode and the second electrode; and
a hole transport region between the first electrode and the emission layer, and comprising a polycyclic compound represented by Formula 1: 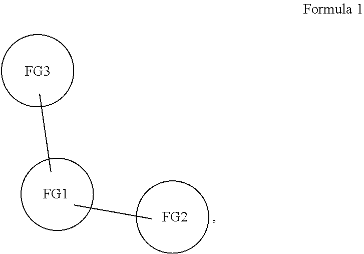
wherein in Formula 1, FG1 is represented by Formula 1-1, FG2 is represented by Formula 1-2, and FG3 is represented by one of Formulas 1-3 to 1-5: 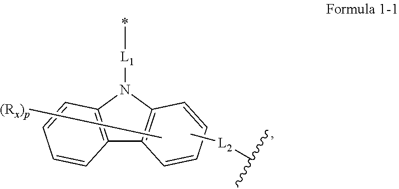
wherein in Formula 1-1,
p is an integer of 0 to 7,
L₁ is a direct linkage or a substituted or unsubstituted arylene group having 6 to 30 ring-forming carbon atoms,
L₂ is a direct linkage or a substituted or unsubstituted arylene group having 6 to 30 ring-forming carbon atoms,
Rₓ is a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
“ [symbol] ” is a portion bonded to FG2, and
“ [symbol] ” is a portion bonded to FG3, 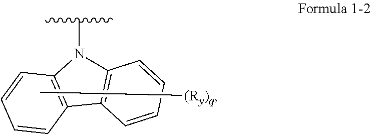
wherein in Formula 1-2,
q is an integer of 0 to 8,
R_{y} is a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and
“ [symbol] ” is a portion bonded to FG1, 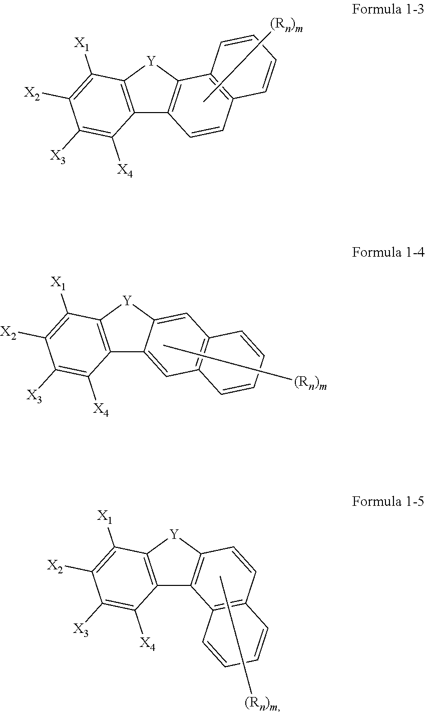
and
wherein in Formulas 1-3 to 1-5,
m is an integer of 0 to 6,
Rₙ is a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
Y is O or S,
X₁ to X₄ are each independently a portion bonded to FG1 of Formula 1, a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and
at least one of X₁ to X₄ is a portion bonded to FG1 of Formula 1.

2. The light emitting element of claim 1, wherein Formula 1 is represented by one of Formulas 2-1 to 2-3: 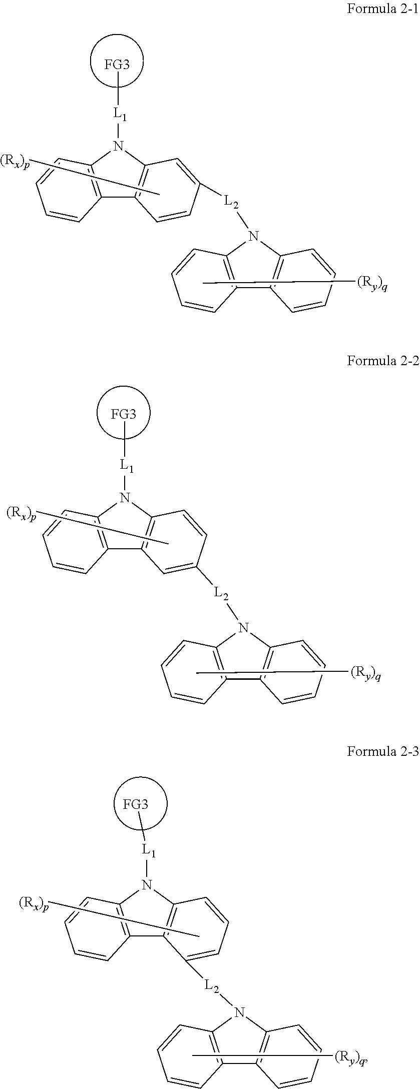
and
wherein in Formulas 2-1 to 2-3, FG3, L₁, L₂, Rₓ, R_{y}, p, and q are as defined in Formula 1.

3. The light emitting element of claim 1, wherein Formula 1 is represented by Formula 3-1 or Formula 3-2: 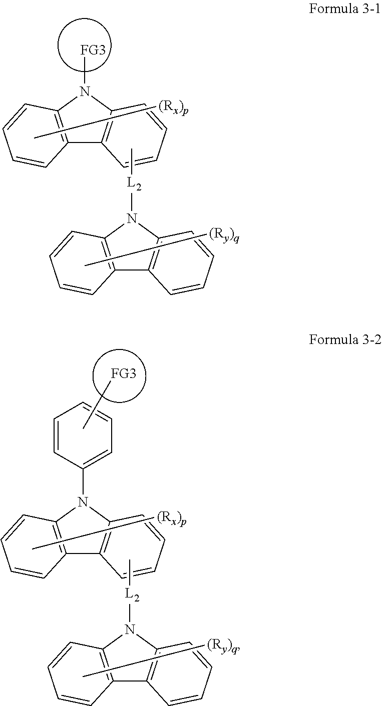
and
wherein in Formulas 3-1 and 3-2, FG3, L₂, Rₓ, R_{y}, p, and q are as defined in Formula 1.

4. The light emitting element of claim 3, wherein Formula 3-2 is represented by Formula 3A or Formula 3B: 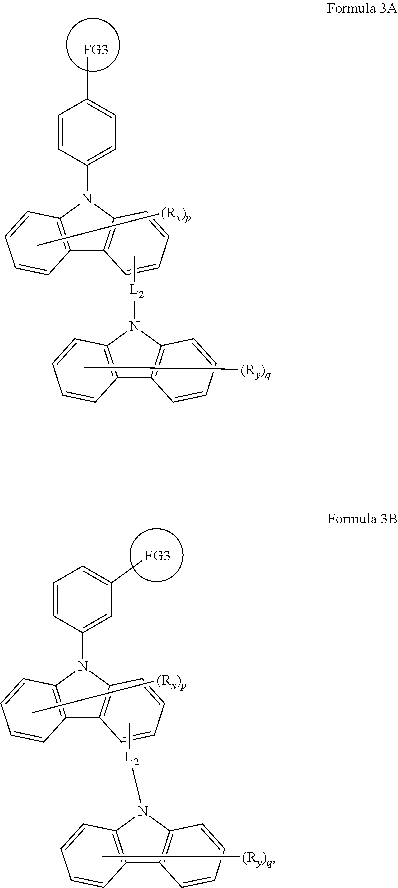
and
wherein in Formulas 3A and 3B, FG3, L₂, Rₓ, R_{y}, p, and q are as defined in Formula 1.

5. The light emitting element of claim 1, wherein Formula 1 is represented by Formula 4-1 or Formula 4-2: 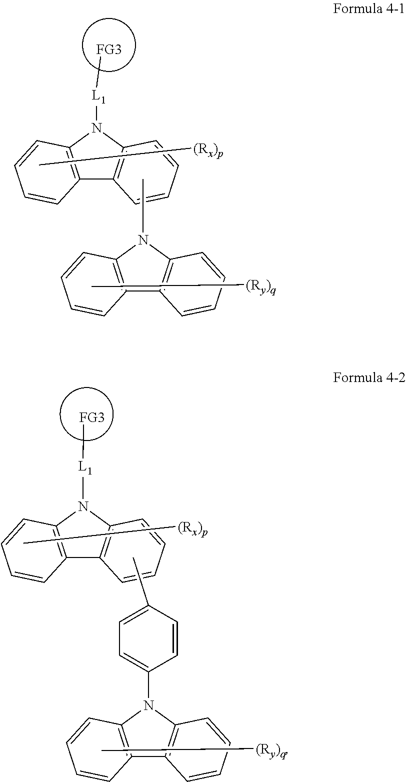
and
wherein in Formulas 4-1 and 4-2, FG3, L₁, Rₓ, R_{y}, p, and q are as defined in Formula 1.

6. The light emitting element of claim 1, wherein in Formula 1-1, Rₓ is a hydrogen atom or an unsubstituted phenyl group.

7. The light emitting element of claim 1, wherein in Formula 1-2, R_{y} is a hydrogen atom, a deuterium atom, a deuterium-substituted phenyl group, an unsubstituted phenyl group, an unsubstituted naphthyl group, an unsubstituted dibenzofuran group, or an unsubstituted dibenzothiophene group.

8. The light emitting element of claim 1, wherein in each of Formulas 1-3 to 1-5, each X₁ to X₄ not bonded to FG1 of Formula 1 is a hydrogen atom.

9. The light emitting element of claim 1, wherein the hole transport region comprises:
a hole injection layer on the first electrode; and
a hole transport layer between the hole injection layer and the emission layer,
the hole transport layer comprising the polycyclic compound.

10. The light emitting element of claim 1, wherein the emission layer is configured to emit blue light.

11. The light emitting element of claim 1, wherein the emission layer is configured to emit light of fluorescence.

12. The light emitting element of claim 1, wherein in Formula 1, FG1 is represented by one of B-1 to B-7:

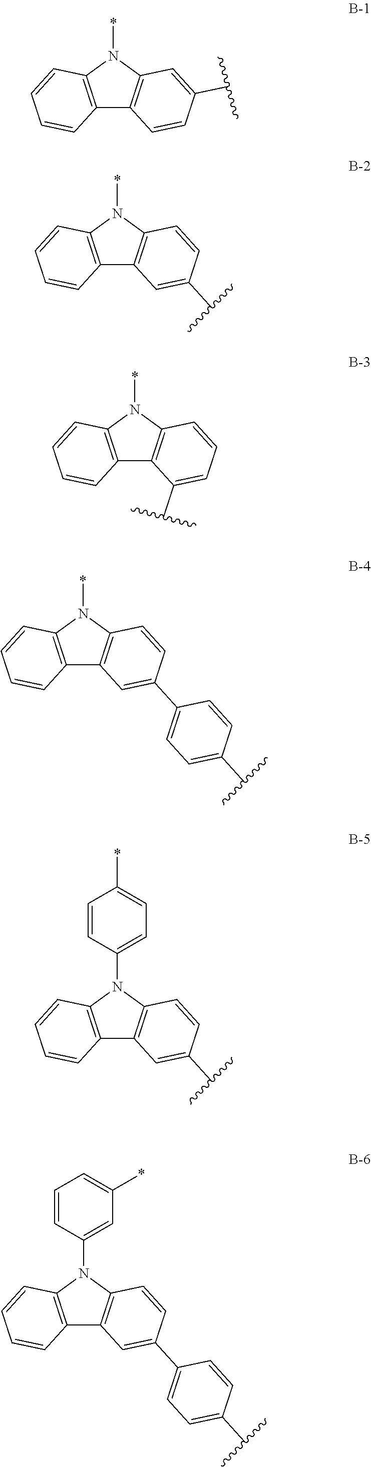 [SMILES: *c1ccc2c3ccccc3n(*)c2c1 ; *c1ccc2c(c1)c1ccccc1n2* ; *c1cccc2c1c1ccccc1n2* ; *c1ccc(-c2ccc3c(c2)c2ccccc2n3*)cc1 ; *c1ccc(-n2c3ccccc3c3cc(*)ccc32)cc1 ; *c1ccc(-c2ccc3c(c2)c2ccccc2n3-c2cccc(*)c2)cc1]

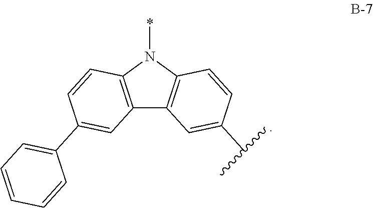 [SMILES: *c1ccc2c(c1)c1cc(-c3ccccc3)ccc1n2*]

13. The light emitting element of claim 1, wherein in Formula 1, FG2 is represented by one of C-1 to C-8:

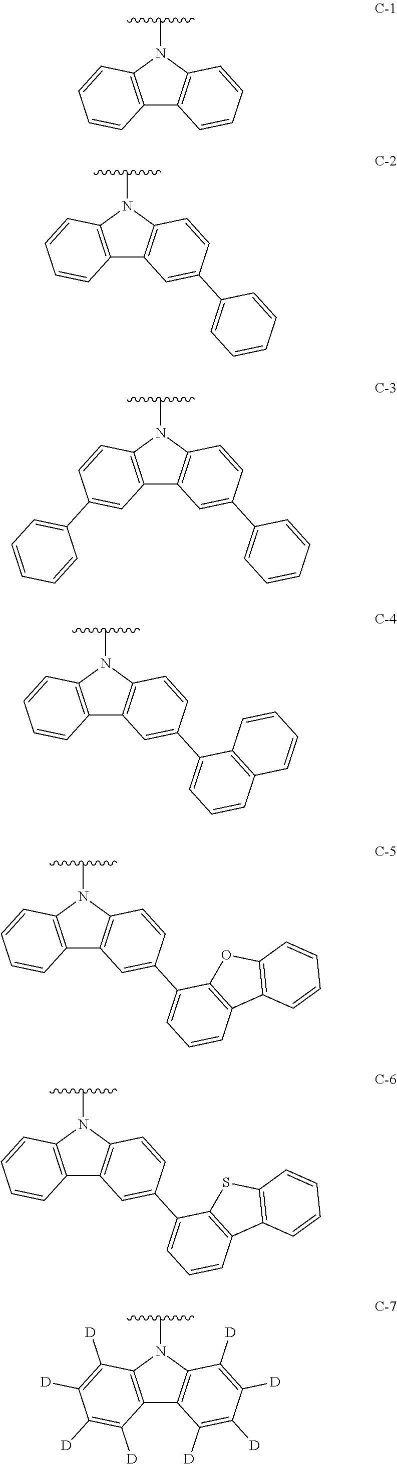

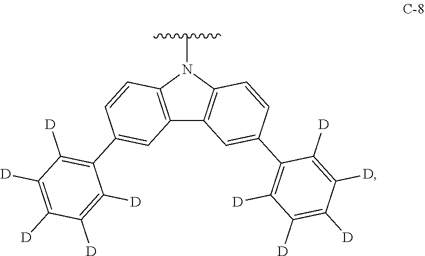

14. The light emitting element of claim 1, wherein in Formula 1, FG1 is represented by one of B-1 to B-7, and
in Formula 1, FG2 is represented by one of C-1 to C-8:

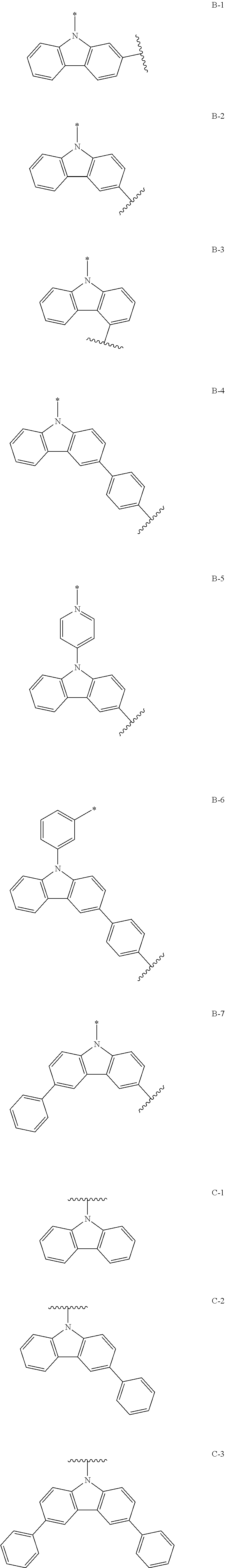

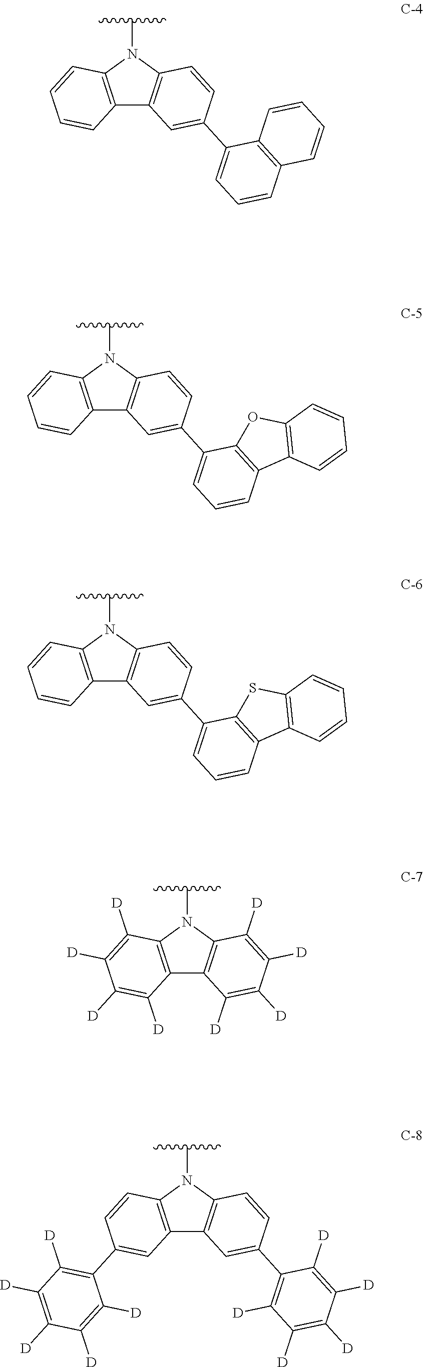 [SMILES: *n1c2ccccc2c2cc(-c3cccc4ccccc34)ccc21 ; *n1c2ccccc2c2cc(-c3cccc4c3oc3ccccc34)ccc21 ; *n1c2ccccc2c2cc(-c3cccc4c3sc3ccccc34)ccc21 ; *n1c2c([2H])c([2H])c([2H])c([2H])c2c2c([2H])c([2H])c([2H])c([2H])c21 ; *n1c2ccc(-c3c([2H])c([2H])c([2H])c([2H])c3[2H])cc2c2cc(-c3c([2H])c([2H])c([2H])c([2H])c3[2H])ccc21]

15. A polycyclic compound represented by Formula 1: 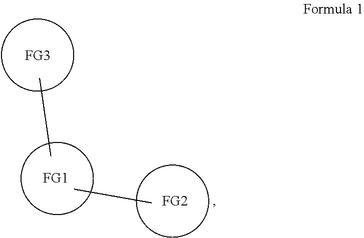
wherein in Formula 1, FG1 is represented by Formula 1-1, FG2 is represented by Formula 1-2, and FG3 is represented by one of Formulas 1-3 to 1-5: 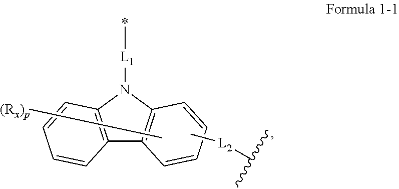
wherein in Formula 1-1,
p is an integer of 0 to 7,
L₁ is a direct linkage or a substituted or unsubstituted arylene group having 6 to 30 ring-forming carbon atoms,
L₂ is a direct linkage or a substituted or unsubstituted arylene group having 6 to 30 ring-forming carbon atoms,
Rₓ is a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
“ [symbol] ” is a portion bonded to FG2, and
“ [symbol] ” is a portion bonded to FG3, 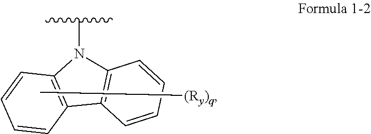
wherein in Formula 1-2,
q is an integer of 0 to 8,
R_{y} is a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and
“ [symbol] ” is a portion bonded to FG1, 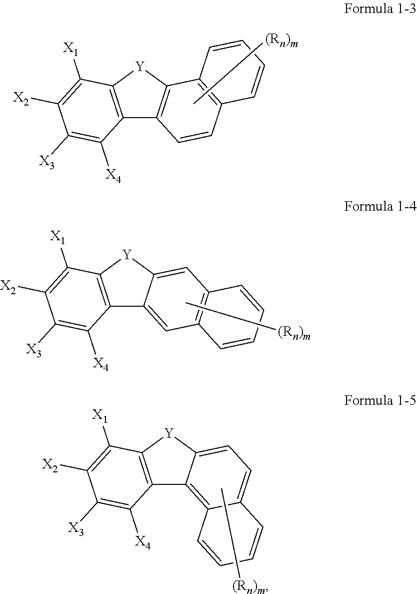
and
wherein in Formulas 1-3 to 1-5,
m is an integer of 0 to 6,
Rₙ is a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
Y is O or S,
X₁ to X₄ are each independently a portion bonded to FG1 of Formula 1, a hydrogen atom, a deuterium atom, a halogen atom, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and
at least one of X₁ to X₄ is a portion bonded to FG1 of Formula 1.

16. The polycyclic compound of claim 15, wherein Formula 1 is represented by one of Formulas 2-1 to 2-3: 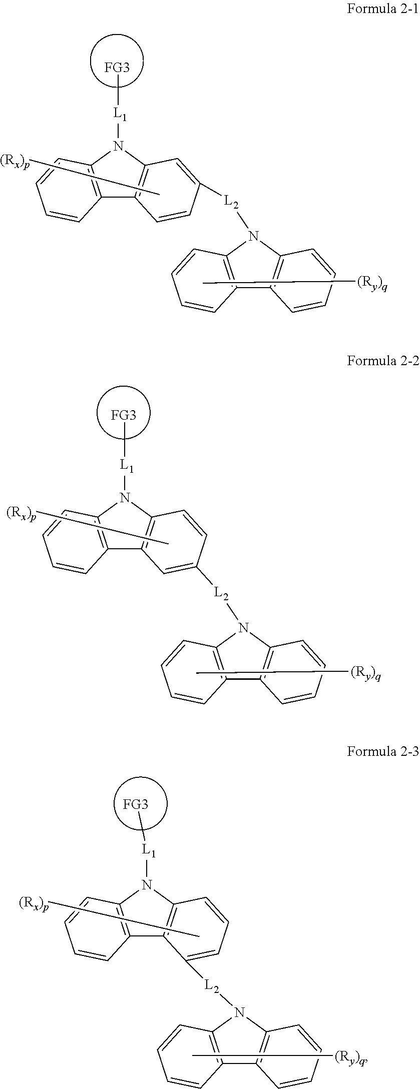
and
wherein in Formulas 2-1 to 2-3, FG3, L₁, L₂, Rₓ, R_{y}, p, and q are as defined in Formula 1.

17. The polycyclic compound of claim 15, wherein Formula 1 is represented by Formula 3-1 or Formula 3-2: 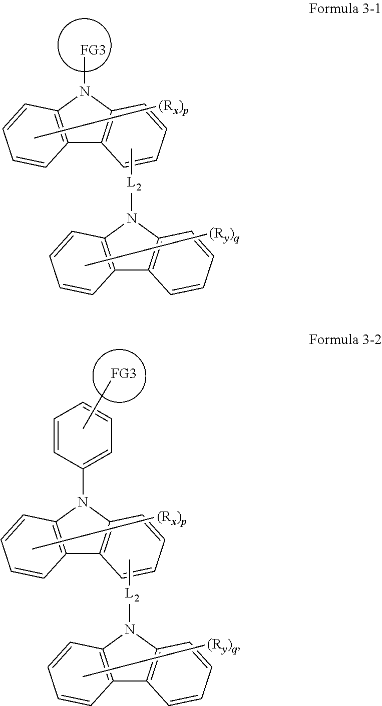
and
wherein in Formulas 3-1 and 3-2, FG3, L₂, Rₓ, R_{y}, p, and q are as defined in Formula 1.

18. The polycyclic compound of claim 17, wherein Formula 3-2 is represented by Formula 3A or Formula 3B: 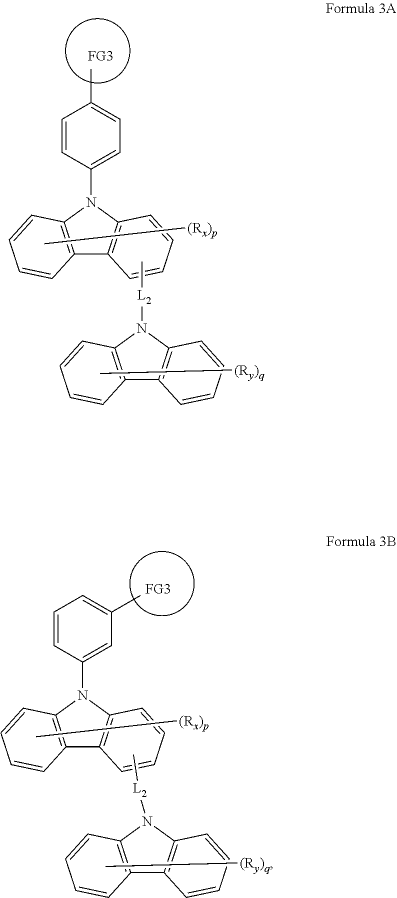
and
wherein in Formulas 3A and 3B, FG3, L₂, Rₓ, R_{y}, p, and q are as defined in Formula 1.

19. The polycyclic compound of claim 15, wherein Formula 1 is represented by Formula 4-1 or Formula 4-2: 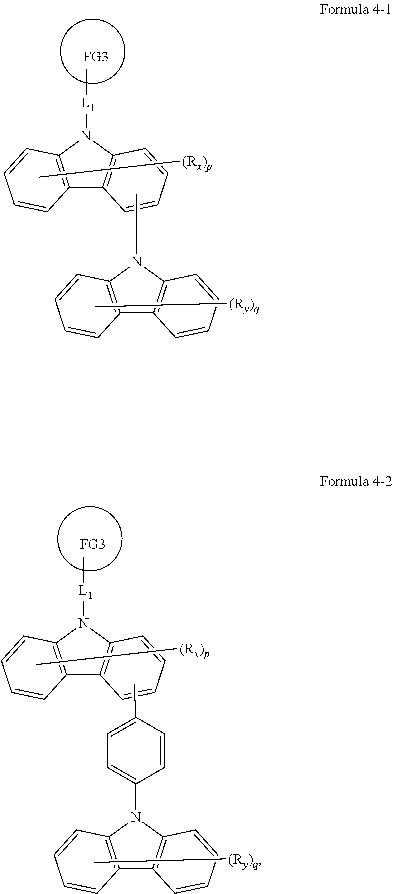
and
wherein in Formulas 4-1 and 4-2, FG3, L₁, Rₓ, R_{y}, p, and q are as defined in Formula 1.

20. The polycyclic compound of claim 15, wherein in Formula 1-1, Rₓ is a hydrogen atom or an unsubstituted phenyl group.

21. The polycyclic compound of claim 15, wherein in Formula 1-2, R_{y} is a hydrogen atom, a deuterium atom, a deuterium-substituted phenyl group, an unsubstituted phenyl group, an unsubstituted naphthyl group, an unsubstituted dibenzofuran group, or an unsubstituted dibenzothiophene group.

22. The polycyclic compound of claim 15, wherein in each of Formulas 1-3 to 1-5, each X₁ to X₄ not bonded to FG1 of Formula 1 is a hydrogen atom.

23. The polycyclic compound of claim 15, wherein in Formula 1, FG1 is represented by one of B-1 to B-7:

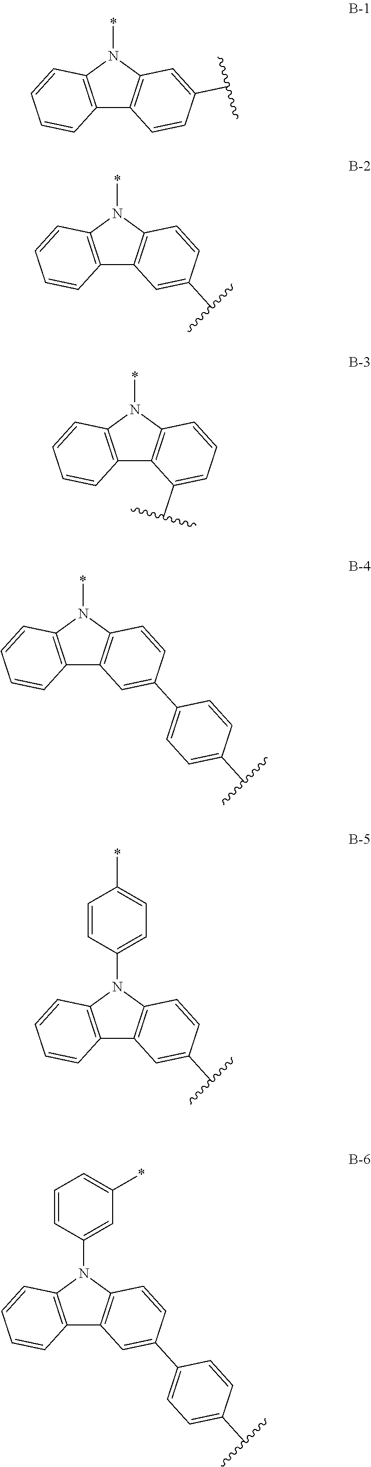 [SMILES: *c1ccc2c3ccccc3n(*)c2c1 ; *c1ccc2c(c1)c1ccccc1n2* ; *c1cccc2c1c1ccccc1n2* ; *c1ccc(-c2ccc3c(c2)c2ccccc2n3*)cc1 ; *c1ccc(-n2c3ccccc3c3cc(*)ccc32)cc1 ; *c1ccc(-c2ccc3c(c2)c2ccccc2n3-c2cccc(*)c2)cc1]

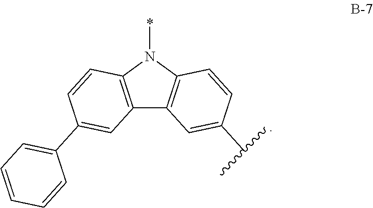 [SMILES: *c1ccc2c(c1)c1cc(-c3ccccc3)ccc1n2*]

24. The polycyclic compound of claim 15, wherein in Formula 1, FG2 is represented by one of C-1 to C-8:

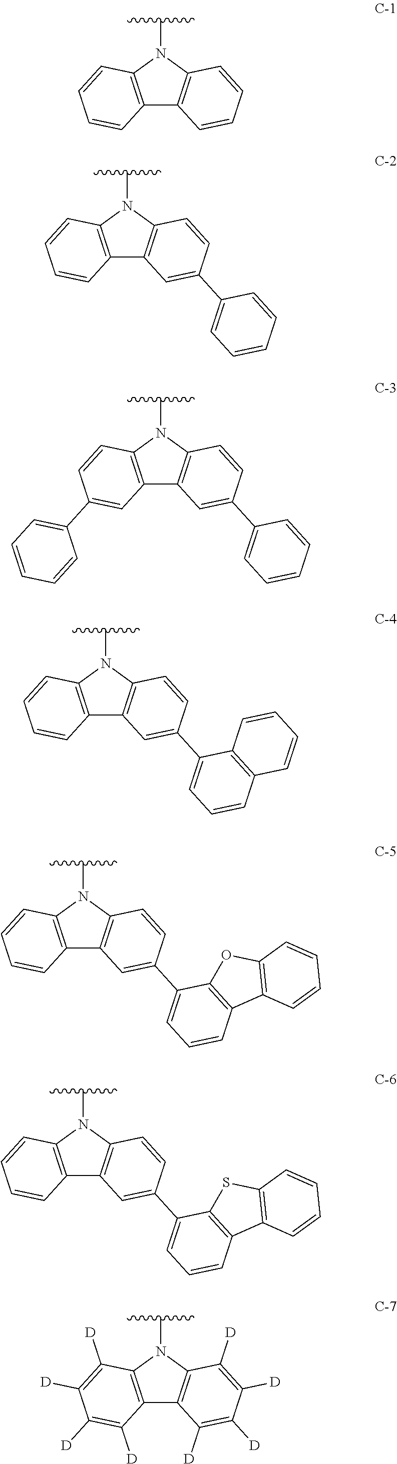

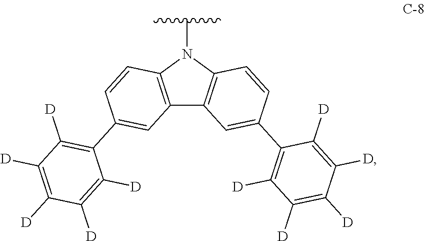

25. The polycyclic compound of claim 15, wherein in Formula 1, FG1 is represented by one of B-1 to B-7, and
in Formula 1, FG2 is represented by one of C-1 to C-8: 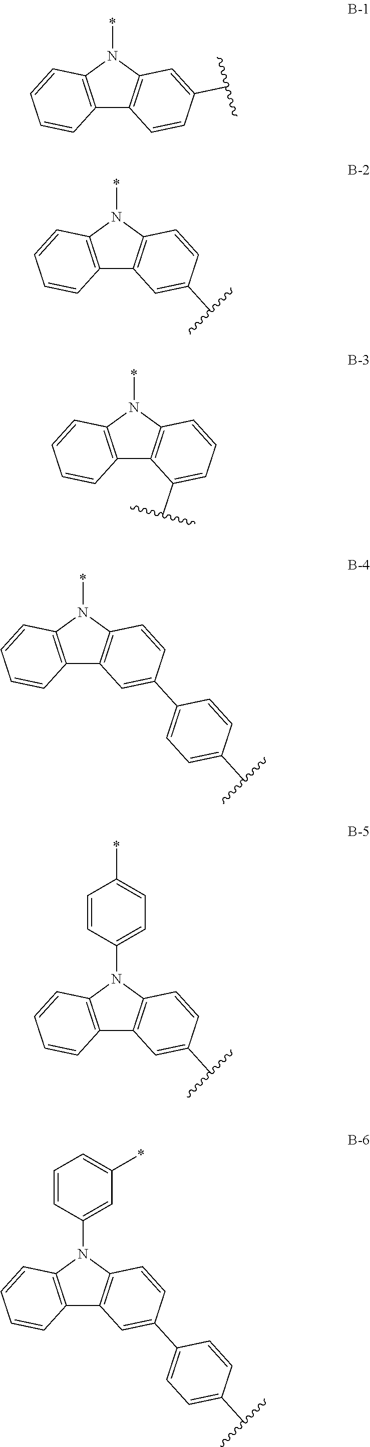 [SMILES: *c1ccc2c3ccccc3n(*)c2c1 ; *c1ccc2c(c1)c1ccccc1n2* ; *c1cccc2c1c1ccccc1n2* ; *c1ccc(-c2ccc3c(c2)c2ccccc2n3*)cc1 ; *c1ccc(-n2c3ccccc3c3cc(*)ccc32)cc1 ; *c1ccc(-c2ccc3c(c2)c2ccccc2n3-c2cccc(*)c2)cc1] 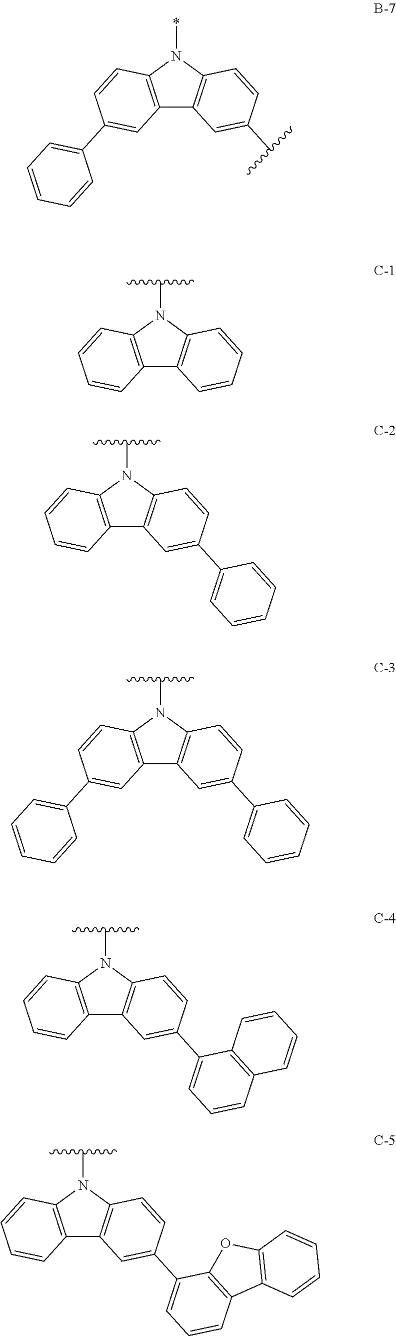 [SMILES: *c1ccc2c(c1)c1cc(-c3ccccc3)ccc1n2* ; *n1c2ccccc2c2ccccc21 ; *n1c2ccccc2c2cc(-c3ccccc3)ccc21 ; *n1c2ccc(-c3ccccc3)cc2c2cc(-c3ccccc3)ccc21 ; *n1c2ccccc2c2cc(-c3cccc4ccccc34)ccc21 ; *n1c2ccccc2c2cc(-c3cccc4c3oc3ccccc34)ccc21] [chemical structure]
